(19) United States
(12) Patent Application Publication
(10) Pub. No.: US 2023/0355737 A1
(43) Pub. Date: Nov. 9, 2023

(54) PARTICLE COMPRISING AN RSV-F PROTEIN FOR USE IN RSV VACCINATION

(71) Applicants: DBV TECHNOLOGIES, MONTROUGE (FR); VIROMETIX AG, SCHLIEREN (CH)

(72) Inventors: PIERRE-LOUIS HERVE, MONTROUGE (FR); LUCIE MONDOULET, CHATILLON (FR); NATHALIE DONNE, ALLAUCH (FR); ARMANDO ZUNIGA, ZÜRICH (CH); ARIN GHASPARIAN, ZÜRICH (CH)

(21) Appl. No.: 18/028,571

(22) PCT Filed: Sep. 24, 2021
(86) PCT No.: PCT/EP2021/076367

§ 371 Date: Mar. 27, 2023

(30) Foreign Application Priority Data
Sep. 28, 2020 (EP) 20306101.5
Jun. 8, 2021 (EP) 21178375.8

(51) Int. Cl. A61K 39/12; A61K 9/70; A61K 9/00

(52) U.S. Cl. CPC A61K 39/12; A61K 9/7023; A61K 9/0014; A61K 2039/55

(57) ABSTRACT

The present invention relates to the epicutaneous vaccination against RSV vaccination with a skin patch device loaded with a particle exposing an RSV-F protein, a variant or a fragment thereof.

The present invention relates to the epicutaneous vaccination against RSV vaccination with a skin patch device loaded with a particle exposing a RSV-F protein, a variant or a fragment thereof.

Respiratory syncytial virus (RSV) is a member of the genus Pneumovirus of the family Paramyxoviridae. RSV is a negative sense, single stranded RNA virus that is the leading cause of serious respiratory tract infections in infants and children, with the primary infection occurring in children from 6 weeks to 2 years of age. Bronchiolitis and pneumonia, the most severe clinical manifestations of RSV, affect 25 to 40% of infected children, leading to hospitalization in 0.5 to 2% of cases (McNamara P S et al., 2002). RSV infection thus constitutes a public health issue along with a substantial economic burden.

The development of RSV vaccines has long been negatively affected by the dramatic outcome of the very first clinical trial, which examined the efficacy of a formalin-inactivated virus vaccine (FI-RSV) in young children. Unexpectedly, this vaccine exacerbated clinical symptoms after infection and led to the hospitalization of almost 80% of the participants. Currently, the only approved approach to prophylaxis of RSV disease is passive immunization by the humanized monoclonal antibody palivizumab (Synagis®) which is specific for an epitope on the F protein. Said antibody is approved for intravenous administration to pediatric patients for prevention of serious lower respiratory tract disease caused by RSV. It is used in the groups of children who are particularly at high risk for this disease, such as children who were born five or more weeks prematurely, children who are less than two years of age and have had treatment for bronchopulmonary dysplasia and children who are less than two years of age and were born with a serious heart disease.

While this antibody efficiently reduces the burden of severe bronchiolitis in at-risk populations, it is expensive and requires several injections throughout RSV season, hence showing unacceptable limitations. Moreover, this antibody presents a limited efficacy against viral replication in upper airways and therefore do not prevents viral transmission.

There is still no marketed vaccine against RSV although many candidates are under clinical evaluation. The development of a vaccine has indeed proven to be problematic. In particular, immunization would be required in the immediate neonatal period since the peak incidence of lower respiratory tract disease occurs at 2-5 months of age. However, it is known that the neonatal immune response is immature at that time. In addition, the infant at that point in time still has high titers of maternally acquired RSV antibody, which might reduce vaccine efficacy.

Two glycoproteins, F and G, on the surface of RSV have been shown to be the targets of neutralizing antibodies. These two proteins are also primarily responsible for viral recognition and entry into target cells; G protein binds to a specific cellular receptor and the F protein promotes fusion of the virus with the cell. The F protein is also expressed on the surface of infected cells and is responsible for subsequent fusion with other cells leading to syncytia formation and cell to cell virus spread.

RSV vaccines currently in clinical trials are designed to generate neutralizing antibodies against epitopes of the RSV fusion glycoprotein (F) that would be able to neutralize RSV infection at an early state (Higgins D et al., 2016). Indeed, RSV-F neutralizing antibodies are the main correlate for protection against RSV infection and a strong correlation has been observed in human between the presence of naturally-acquired RSV-neutralizing antibodies and protection (Piedra P et al., 2003).

While efforts were made, there is still a long-time felt and unfulfilled need for an effective and safe therapeutic agent in the prevention of RSV infection.

### SUMMARY OF THE INVENTION

The invention harnesses the power of epicutaneous immunotherapy and provides a novel therapeutic and prophylactic approach to RSV infection.

In a first aspect, the invention relates to a particle comprising an RSV-F protein, a variant or a fragment thereof for use in a method of prevention of a disease caused by a Respiratory syncytial virus (RSV) (also called herein RSV-associated disease) by epicutaneous vaccination with said particle.

In a second aspect, the invention relates to a particle comprising an RSV-F protein for use in a method for vaccinating an infant against RSV by maternal epicutaneous vaccination with said particle. Preferably, said particle is a synthetic virus-like-particle (SVLP). Preferably, said particle is applied using a skin patch device, preferably an electrostatic skin patch device. In a further aspect, the present invention provides a conjugate comprising (a) a lipopeptide building block and (b) an RSV-F protein, a variant or a fragment thereof, wherein said lipopeptide building block consists of (i) a peptide moiety comprising at least one coiled coil peptide chain segment, and (ii) a lipid moiety comprising two or three, preferably two hydrocarbyl chains; wherein said RSV-F protein, said variant or said fragment thereof is conjugated, directly or via a linker, to said lipopeptide building block. In a certain aspect, the peptide moiety further comprises a T helper cell epitope.

In a third aspect, the invention relates to a method for preparing a skin patch device comprising depositing, preferably by electrospraying, at least one particle comprising an RSV-F protein, a variant or a fragment thereof on the surface of a skin patch device.

In a fourth aspect, the invention relates to a skin patch device comprising an application surface, wherein the application surface contains an SVLP comprising an RSV-F protein, a variant or a fragment thereof.

### DETAILED DESCRIPTION OF THE INVENTION

The inventors have shown that epicutaneous application of particles comprising an RSV-F protein, a variant or a fragment thereof can be used as a potent pediatric vaccine approach against RSV infection.

Indeed, as illustrated in the Example section, the Inventors showed that the epicutaneous delivery of a synthetic virus-like-particle (SVLP) displaying multiple RSV F-protein site II (FsII, palivizumab epitope) mimetic as antigen was able to induce and boost anti-RSV neutralizing antibodies. In particular, the Inventors showed that the epicutaneous immunization with SVLPs of the invention, such as SVLP comprising conjugate V-306 (V-306 SVLP), efficiently boosts preexisting immunity induced by the homologous SVLPs administered subcutaneously. This boosting was characterized by a significant increase in F- and FsII-specific antibodies capable of competing with palivizumab for its target antigen and neutralize RSV.

Of note, the epicutaneous immunization was shown to protect against RSV infection as evidenced by the low viral load found in lungs of epicutaneously immunized mice exposed to the virus by intranasal challenge. The inventors further showed that the orientation of the immune response recalled by RSV infection in mice immunized by epicutaneous route was Th1 orientated. Of note, the local expressions of IFN-γ and IL-2, which are Th-1 related cytokines, were higher in epicutaneous immunized mice than in subcutaneous immunized mice. Such results support that the epicutaneous route may avoid exacerbated lung inflammation and is more advantageous than the subcutaneous one to enhance Th1 immunity in the context of RSV immunization.

Thus, epicutaneous immunization with SVLP bearing RSV antigens provides an efficient and safe strategy for providing RSV vaccination, especially in the context of a boosting vaccination. By promoting a Th1-orientated immune response, the epicutaneous immunization of the invention is expected to avoid an exacerbation of clinical symptoms after RSV infection as observed with formalin-inactivated vaccine administered by subcutaneous route.

Because the vaccination is performed via non-invasive and painless epicutaneous route, it also presents a better acceptability for the vaccination of sensible population such as infants, pregnant women or the elderly.

In a first aspect, the invention thus relates to a particle comprising an RSV-F protein, a fragment or variant thereof for use in a method of prevention of a disease caused by RSV by epicutaneous vaccination with said particle.

The invention may be used to protect against RSV infection or diseases caused by RSV or RSV-associated diseases, hence inhibiting or reducing the rate of infection upon exposure of the subject to RSV. Protecting against a RSV disease includes suppressing the onset of bronchiolitis. Therefore, the invention also relates to a particle comprising an RSV-F protein a fragment or variant thereof for use in a method of vaccination against RSV infection or a diseases caused by RSV by epicutaneous vaccination with said particle.

In a more general aspect, the Invention relates to the use of a RSV-F antigen for use in the vaccination against RSV, wherein the RSV-F antigen is administered by epicutaneous route, preferably by means of a skin patch. The RSV-F antigen is typically exposed on the surface of a particle, such as a synthetic virus-like-particle (SVLP) as defined below.

Throughout this specification and the claims, which follow, unless the context requires otherwise, the term “comprise” and its variations cover the term “consisting of” and are to be understood as a non-exhaustive wording and imply the inclusion of a stated feature or element but not the exclusion of any other feature or element. As used in this specification and the appended claims, the singular forms “a”, “an”, and “the” include plural referents, unless the content clearly dictates otherwise.

A protein, peptide or peptide moiety, as defined herein, is any peptide-bond-linked chain of amino acids, regardless of length, secondary and tertiary structure, number of subunits or posttranslational modification. Thus, the terms “peptide”, “polypeptide”, “protein”, “amino acid chain” and “polypeptide chain” are interchangeably used herein. Amino acids included in the peptide of the invention are proteinogenic, non-proteinogenic and synthetic amino acids. Peptides of the invention may include at least one chemical modification, such as lipidation, glycosylation and phosphorylation. Peptides, as understood herein, especially peptides of the invention, are isolated or, preferably can be produced by chemical synthesis, RNA translation and/or recombinant processes.

The term “antigen” as used herein, should refer to molecules capable of being bound by an antibody. The antigen may comprise a peptide, a protein or an epitope mimetic having one or more B-cell epitopes that are to be used to elicit an antigen-specific humoral immune response in an animal. Alternatively, the antigen may comprise a hapten or a carbohydrate. Suitable peptide and protein antigens comprise up to 150 amino acids and include glycopeptides and glycoproteins.

The term “cyclic peptide”, as used herein, refers to a peptide in which the amino acid chain forms at least one ring structure by a covalent bond. The cyclic peptide of the invention comprises two ring structures each formed by a disulfide bond: Side chains of cysteines C4 and C25 are linked forming a first disulfide bond, and side chains of cysteines C8 and C21 are linked forming a second disulfide bond.

The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids. The term “amino acid”, as used herein, also includes unnatural amino acids that are chemically synthesized; alpha- (α-), beta- (β-), gamma- (γ-) and delta- (δ-) etc. amino acids as well as mixtures thereof in any ratio; and, if applicable, any isomeric form of an amino acid, i.e. its D-stereoisomers (labelled with a lower-case initial letter) and L-stereoisomers (labelled with a capital initial letter) (alternatively addressed by the (R) and (S) nomenclature) as well as mixtures thereof in any ratio, preferably in a racemic ratio of 1:1.

Amino acids in this invention are preferably in L-configuration, unless mentioned specifically as D-configuration. The term “deletion” refers herein to a position in an amino acid sequence that is not occupied by an amino acid.

For instance, norleucine (hereunder Nle), allo-isoleucine, D-alanine, ornithine and 2,4-diaminobutyric acid (also called hereunder Dab or Dbu) are encompassed by the wording “amino acid”, in the context of the present invention.

The term “N-terminus”, as used herein, refers to an end of a peptide having a free (—NH₂) or modified amino or amine group. The term “C-terminus”, as used herein, refers to an end of a peptide having a free (—COOH) or modified carboxyl group.

As used therein, “epicutaneous administration or application” designates the application of an antigen on a surface of the skin of a subject under conditions allowing a contact with the surface of the skin. Epicutaneous administration typically comprises skin application under condition sufficient to allow penetration or diffusion of the antigen in the superficial layer(s) of the skin, preferably in the epidermis layers, and/or contact of said antigen with immune cells. As explained further below, epicutaneous administration can be performed by several skin devices, such as skin patch.

“Respiratory syncytial virus” or “RSV” is a member of the genus Pneumovirus of the family Paramyxoviridae. This virus has a genome comprised of a single strand negative-sense RNA, which is tightly associated with viral protein to form the nucleocapsid. The viral envelope is composed of a plasma membrane derived lipid bilayer that contains virally encoded structural proteins. A viral polymerase is packaged with the virion and transcribes genomic RNA into mRNA. The RSV genome encodes three transmembrane structural proteins, F, G, and SH, two matrix proteins, M and M2, three nucleocapsid proteins N, P, and L, and two nonstructural proteins, NS1 and NS2.

As used herein, the term “diseases caused by RSV” includes but is not limited to infections of the lungs and breathing passages with RSV including respiratory distress, bronchiolitis, tracheobronchitis, pneumonia and middle ear infection.

“Respiratory Syncytial Virus-F protein”, also referred to as “RSV-F” or “protein F of RSV” is a type I transmembrane surface protein, which has an N terminal cleaved signal peptide and a membrane anchor near the C terminus. The RSV-F protein is synthesized as an inactive 67 KDa precursor denoted as F0. Unless stated otherwise, the term RSV-F protein the mature RSV-F protein and precursors of the RSV-F protein. The F0 protein is activated proteolytically in the Golgi complex by a furin-like protease at two sites, yielding two disulfide linked polypeptides, F2 and F1, from the N and C terminal, respectively. The RSV-F protein plays a role in fusion of the virus particle to the cell membrane, and is expressed on the surface of infected cells, thus playing a role in cell to cell transmission of the virus and syncytia formation. The amino acid sequence of a preferable and typical RSV-F protein is provided in GenBank as accession number AAX23994 and is also referred to herein as SEQ ID NO: 1.

As used herein, the expression “particle of the invention” refers to a particle comprising, preferably exposing, an RSV-F protein, a variant or a fragment thereof. In a preferred embodiment, said particle comprising an RSV-F protein is a particle exposing an RSV-F protein, typically exposing an RSV-F protein on the surface of said particle. Preferably, said particle comprises at least two copies of protein F of RSV, a variant or a fragment thereof. More preferably, said particle comprises at least 5, at least 10, at least 20, at least 30, at least 40, at least 50, at least 60 copies of protein F of RSV, a variant or a fragment thereof. In a preferred embodiment, said particles comprises about 70 copies of protein F of RSV, a variant or a fragment thereof.

As used herein, a “synthetic virus-like-particle (SVLP)” refers to a self-assembly of components arranged in a repetitive fashion into nanoparticles which bear multiple copies of an antigen, epitope or epitope mimetic of choice such as synthetic antigen mimetics (SAMs), glycopeptides, haptens and small synthetic proteins for multivalent display and delivery to immunocompetent cells. Preferably, the nanoparticles bear multiple copies of synthetic antigen mimetics which is a variant or a fragment of an RSV-F protein, preferably comprising SEQ ID NO: 44.

Typically, the components of the SVLP are lipopeptides which are conjugated directly or via a linker to an antigen, epitope or epitope mimetic of interest.

The diameter of the SVLP is generally less than 100 nm, preferably less than 50 nm, e.g. such as from 10 to 40 nm or from 20 to 30 nm.

As used herein, “a conjugate” refers to a lipopeptide which is conjugated or directly linked to an antigen, epitope or epitope mimetic of interest. Said conjugate is able to self-assemble into nanoparticles. Typically, the conjugate comprises (a) a lipopeptide building block and (b) an RSV antigen. In the context of the invention, the lipopeptide building block comprises (i) a peptide moiety comprising at least one coiled coil peptide chain segment, and (ii) a lipid moiety comprising two or three, preferably two hydrocarbyl chains.

Typically, the coiled-coil peptide chain segment present in the peptide moiety contains multiple tandem repeat motifs, which promotes self-association as fully described further below. The peptide moiety may further comprise a T helper cell epitope, in particular as described further below.

Typically, the hydrocarbyl chain refers to straight alkyl or alkenyl group of at least 7 carbon atoms, for example straight alkyl or alkenyl consisting of between 8 and 50 C atoms, preferably between 8 and 25 C atoms.

The RSV antigen, epitope or epitope mimetic is typically selected from an RSV-F protein, a variant or a fragment thereof, preferably an RSV-F protein variant including cyclic RSV-F protein variants, e.g., as described below. In particular, said cyclic RSV-F protein variant comprises or consists of cyclic peptide of SEQ ID NO: 44, preferably a sequence selected of SEQ ID NO: 45-88, again more preferably SEQ ID NO: 45-64, 84 and 85; again more preferably SEQ ID NO: 45-49, 84 and 85, most preferably SEQ ID NO: 45, 84 and 85.

Unless stated otherwise, or implicit from the context, the expression “the antigen of the invention” refers herein to an RSV-F protein, a variant or a fragment thereof. In a preferred embodiment, said antigen is a recombinant F protein of RSV, a variant or a fragment thereof. In a specific embodiment, said antigen is an epitope mimetic. As used herein, an “epitope mimetic” is a molecule mimicking a natural peptidic or carbohydrate epitope, including peptidic compounds containing one or more non-natural amino acids, e.g. D-amino acids, β-amino acids, γ-amino acids, δ-amino acids, or ε-amino acids, and other replacements known in the art of epitope mimics. Preferred are conformational constrained peptidomimetics, which are fixed in a protein-like conformation. The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, may have specific three-dimensional structural characteristics, and/or specific charge characteristics.

As used herein, a “fragment” of RSV-F protein refers to a portion of the RSV-F protein. Preferably, said fragment comprises between 10 and 250 amino acids, preferably between 20 and 150 amino acids, more preferably between 10 and 80 amino acids, again more preferably between about 10 and 50 amino acids, again more preferably between about 10 and 40 amino acids, most preferably between about 10 and 30 amino acids. Preferably, said fragment includes an immunodominant epitope of the RSV-F protein, typically epitopes recognized by neutralizing antibodies. Said epitopes include but are not limited to:
the antigenic site II of RSV-F protein (also called site A) which includes residues 255 to 275 of RSV-F protein and is the target of palivizumab; and
the antigenic site IV of RSV-F protein (also called site C) includes residues 422 to 438 and is the target of antibody MAb19.

Thus, in a preferred embodiment, said fragment being preferably selected from the group consisting of sequences depicted in SEQ ID NO: 2, 3, 4, 5 and variants thereof.

Said sequences are detailed in the following table:

| Origin | Sequence | SEQ ID NO |
|---|---|---|
| Antigenic Site II | NSELLSLINDMPITNDQKKLMSNN | SEQ ID NO: 2 |
| | SELLSLINDMPITNDQKKLMS | SEQ ID NO: 3 |
| | NSELLSLINDMPITNDQKKLMS | SEQ ID NO: 4 |
| Antigenic site IV | CTASNKNRGIIK | SEQ ID NO: 5 |

Preferably, said sequence is SEQ ID NO: 2 (NSELLSLINDMPITNDQKKLMSNN (positions 254-277 of the glycoprotein RSV-F). Said fragment is the epitope recognized by the palivizumab.

As used herein, the terms “variant” refers to a protein which includes a modification that alters the structure of the RSV-F protein but which retains the immunological properties of the F protein such that an immune response generated against an F protein variant will recognize the native F protein. A variant and the RSV-F protein, as disclosed in SEQ ID NO: 1, may differ in amino acid sequence by one or more substitutions, deletions, additions, fusions and truncations that may be conservative or non-conservative and may be present in any combination. For example, variants may be those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Cyclic Peptide

Most preferably, said antigen of the invention is a variant of the RSV-F protein, wherein said variant is a cyclic peptide comprising an amino acid sequence (I), wherein said amino acid sequence (I) comprises, preferably consists of, the amino acid sequence: X1-X2-X3-C4-X5-X6-X7-C8-X9-X10-X11-P12-I13-T14-N15-D16-Q17-K18-K19-L20-C21-X22-X23-X24-C25-X26-X27-X28-X29-X30 (SEQ ID NO: 44), wherein X1, X2, X3, X5, X6, X7, X9, X10, X11, X22, X23, X24, X26, X27, X28 and X29 are independently of each other an amino acid; C4, C8, C21 and C25 are independently of each other cysteine; P12 is proline; 113 is isoleucine; T14 is threonine; N15 is asparagine; D16 is aspartic acid; Q17 is glutamine; K18 and K19 are independently of each other lysine; L20 is leucine; and X30 is an amino acid or a deletion, wherein said cysteines C4 and C25 form a first disulfide bond and said cysteines C8 and C21 form a second disulfide bond.

Said cyclic peptides were produced using automated solid-phase peptide synthesis, wherein said disulfide bonds between cysteines C4 and C25 and cysteines C8 and C21 were obtained by oxidative refolding resulting in a beneficial spatial conformation.

In a preferred embodiment, said cyclic peptide has a length of at most 80 amino acids. In a further preferred embodiment, said cyclic peptide has a length of at most 60 amino acids. In a further preferred embodiment, said cyclic peptide has a length of at most 40 amino acids. In a further preferred embodiment, said cyclic peptide has a length of at most 30 amino acids.

In a preferred embodiment, said X11 is selected from norleucine, norvaline, methionine, wherein preferably X11 is norleucine (Nle).

In a preferred embodiment, said X23 is selected from glutamine, glycine, asparagine or serine. In a preferred embodiment, said X23 is asparagine or serine.

In a preferred embodiment, X24 is selected from lysine, 2,4-diaminobutyric acid, asparagine, ornithine, glutamine, glycine or serine or aspartic acid. In another preferred embodiment, X24 is selected from lysine, 2,4-diaminobutyric acid, aspartic acid or asparagine. In another preferred embodiment, X24 is selected from asparagine, lysine, ornithine, 2,4-diaminobutyric acid (Dab), glutamine, glycine or serine.

In another more preferred embodiment, X11 is norleucine, X24 is selected from serine, glutamine, glycine, 2,4-diaminobutyric acid, lysine or asparagine, more preferably X24 is selected from serine, glutamine, glycine, 2,4-diaminobutyric acid or lysine and said C-terminal amino acid of said amino acid sequence (I) is alanine, preferably D-alanine.

In another preferred embodiment, said X1 is a polar or hydrophobic amino acid. In another more preferred embodiment, X1 is asparagine or leucine. In another preferred embodiment, said X1 is selected from asparagine, glutamine, leucine, serine, or glycine.

In another preferred embodiment, said X1, X23 and X24 are each independently selected from the group consisting of ornithine, aspartic acid, lysine, asparagine, 2,4-diaminobutyric acid (Dab), glutamine, leucine, serine, and glycine. In another preferred embodiment, said X1, X23 and X24 are each independently selected from the group consisting of asparagine, glutamine, serine, and glycine. In another preferred embodiment, said X1, X23 and X24 are each independently selected from the group consisting of glutamine, serine, and glycine

In another preferred embodiment, said X2, X6 and X22 are independently of each other a polar amino acid. More preferably, X2, X6 and X22 are independently of each other serine.

In another preferred embodiment, said X3 is an amino acid having an acidic or negatively charged side chain at a physiological pH (about pH 7). Preferably, X3 is glutamate.

In another preferred embodiment, said X5 and X7 are independently of each other a hydrophobic amino acid. Preferably, X5 and X7 are independently of each other selected of leucine, alloleucine, alloisoleucine, homoleucine, isoleucine, 2-aminobutyric acid, norleucine, norvaline or valine. In another more again preferred embodiment, X5 and/or X7 are leucine.

In another preferred embodiment, said X9 and X23 are independently of each other a polar amino acid. Preferably, X9 and/or X23 is asparagine. In another again more preferred embodiment, X9 and X23 are asparagine. Preferably, X9 and X23 are independently of each other selected of asparagine, glutamine, serine or glycine.

In another preferred embodiment, said X10 is an amino acid having an acidic or negatively charged side chain at a physiological pH (about pH 7). Preferably, X10 is aspartic acid.

In another preferred embodiment, said X26 is a hydrophobic or polar amino acid. Preferably, X26 is selected of leucine, 2-aminobutyric acid, norleucine, norvaline, valine, or asparagine. More preferably, X26 is leucine or glutamine.

In another preferred embodiment, said X27 is a polar or hydrophobic amino acid or an amino acid having an acidic or negatively charged side chain at a physiological pH (about pH 7). Preferably, X27 is selected of selected of serine, threonine; leucine, valine; diaminobutyric acid, 2,3-diaminopropanoic acid, lysine, or ornithine. More preferably, X27 is serine, isoleucine, or lysine.

In another preferred embodiment, said X28 is a polar or hydrophobic amino acid. Preferably, X28 is selected of, serine, threonine; leucine, 2-aminobutyric acid, or valine. More preferably, X28 is valine or serine.

In another preferred embodiment, said X29 is a hydrophobic amino acid or an amino acid having a negatively charged side chain at physiological pH (about pH 7). Preferably, X29 is selected of the D- or L-stereoisomer, preferably the D-stereoisomer of 2-aminobutyric acid, 2-aminoheptanoic acid, alanine, leucine, valine; or arginine. In another preferred embodiment, X29 is D- or L-alanine or D- or L-arginine.

In another preferred embodiment, said X30 is a deletion or a hydrophobic or polar D- or L-amino acid, preferably X30 is a hydrophobic or polar amino acid D-amino acid. Preferably, X30 is a deletion or X30 is selected of the D- or L-stereoisomer, preferably the D-stereoisomer of 2-aminobutyric acid, 2-aminoheptanoic acid, alanine, leucine, valine; or asparagine. In another preferred embodiment, X30 is D- or L-glutamine or D- or L-alanine. In another more preferred embodiment, X30 is D-glutamine or D-alanine.

In another preferred embodiment, said C-terminal amino acid of said amino acid sequence (I) is selected from serine, glutamine, glycine alanine, leucine, valine, norleucine, norvaline, isoleucine, homoleucine, vinylglycine, 2-aminobutyric acid, 2-allylglycine, alloleucine, alloisoleucine, or 2-aminoheptanoic acid. In a certain embodiment, the C-terminal amino acid of said amino acid sequence (I) is a D-amino acid, preferably said C-terminal amino acid is selected from D-alanine, D-leucine, D-valine, D-norleucine, D-norvaline, D-isoleucine, D-homoleucine, D-vinylglycine, D-2-aminobutyric acid, D-2-allylglycine, D-alloleucine D-alloisoleucine, or D-2-aminoheptanoic acid.

In another very preferred embodiment, said amino acid sequence of SEQ ID NO: 44 is an amino acid selected from: Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Dab-Cys-Gln-Ser-Val-Arg-ala (SEQ ID NO: 45), Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Lys-Cys-Gln-Ser-Val-Arg-ala (SEQ ID NO: 46), Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Asn-Cys-Gln-Ser-Val-Arg-ala (SEQ ID NO: 47), Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Asp-Cys-Gln-Ser-Val-Arg-ala (SEQ ID NO: 48) or Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Orn-Cys-Gln-Ser-Val-Arg-ala (SEQ ID NO: 49).

In another very preferred embodiment, said amino acid sequence (I) is selected from SEQ ID NO: 44, 45, 46, 47, 48 49, Arg-Leu-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Asn-Cys-Leu-Lys-Ser-ala (SEQ ID NO: 50), Pro-Val-Ser-Thr-Tyr-Met-Leu-Thr-Asn-Ser-Glu-Cys-Leu-Ser-Leu-Cys-Asn-Asp-Met-Pro-Ile-Thr-Asn-Asp-Gln-Lys-Lys-Leu-Cys-Ser-Asn-Asn-Cys-Gln-Ile-Val-Arg-Gln-Gln-ala (SEQ ID NO: 51), Arg-Leu-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Lys-Cys-Leu-Lys-Ser-ala (SEQ ID NO: 52), Arg-Leu-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Dab-Cys-Leu-Lys-Ser-ala (SEQ ID NO: 53), Pro-Val-Ser-Thr-Tyr-Met-Leu-Thr-Asn-Ser-Glu-Cys-Leu-Ser-Leu-Cys-Asn-Asp-Met-Pro-Ile-Thr-Asn-Asp-Gln-Lys-Lys-Leu-Cys-Ser-Asn-Lys-Cys-Gln-Ile-Val-Arg-Gln-Gln-ala (SEQ ID NO: 54), Pro-Val-Ser-Thr-Tyr-Met-Leu-Thr-Asn-Ser-Glu-Cys-Leu-Ser-Leu-Cys-Asn-Asp-Met-Pro-Ile-Thr-Asn-Asp-Gln-Lys-Lys-Leu-Cys-Ser-Asn-Dab-Cys-Gln-Ile-Val-Arg-Gln-Gln-ala (SEQ ID NO: 55), Arg-Leu-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Asp-Cys-Leu-Lys-Ser-ala (SEQ ID NO: 56), Arg-Leu-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Orn-Cys-Leu-Lys-Ser-ala (SEQ ID NO: 57), Pro-Val-Ser-Thr-Tyr-Met-Leu-Thr-Asn-Ser-Glu-Cys-Leu-Ser-Leu-Cys-Asn-Asp-Met-Pro-Ile-Thr-Asn-Asp-Gln-Lys-Lys-Leu-Cys-Ser-Asn-Asp-Cys-Gln-Ile-Val-Arg-Gln-Gln-ala (SEQ ID NO: 58), Pro-Val-Ser-Thr-Tyr-Met-Leu-Thr-Asn-Ser-Glu-Cys-Leu-Ser-Leu-Cys-Asn-Asp-Met-Pro-Ile-Thr-Asn-Asp-Gln-Lys-Lys-Leu-Cys-Ser-Asn-Orn-Cys-Gln-Ile-Val-Arg-Gln-Gln-ala (SEQ ID NO: 59), Pro-Val-Ser-Thr-Tyr-Met-Leu-Thr-Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Asn-Cys-Gln-Ile-Val-Arg-Gln-Gln-ala (SEQ ID NO: 60), Pro-Val-Ser-Thr-Tyr-Met-Leu-Thr-Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Lys-Cys-Gln-Ile-Val-Arg-Gln-Gln-ala (SEQ ID NO: 61), Pro-Val-Ser-Thr-Tyr-Met-Leu-Thr-Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Dab-Cys-Gln-Ile-Val-Arg-Gln-Gln-ala (SEQ ID NO: 62), Pro-Val-Ser-Thr-Tyr-Met-Leu-Thr-Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Asp-Cys-Gln-Ile-Val-Arg-Gln-Gln-ala (SEQ ID NO: 63), or Pro-Val-Ser-Thr-Tyr-Met-Leu-Thr-Asn-Ser-Glu-Cys-Leu-Ser-Leu-Cys-Asn-Asp-Nle-Pro-Ile-Thr-Asn-Asp-Gln-Lys-Lys-Leu-Cys-Ser-Asn-Orn-Cys-Gln-Ile-Val-Arg-Gln-Gln-ala (SEQ ID NO: 64).

In another very preferred embodiment, said amino acid sequence of SEQ ID NO: 44 is selected from any one of SEQ ID NO: 45-64,

SEQ ID NO: 65:
NSECLSLCND-Nle-PITNDQKKLCSS-Dab-CQSVRa,
SEQ ID NO: 66:
NSECLSLCND-Nle-PITNDQKKLCSSNCQSVRa,
SEQ ID NO: 67:
NSECLSLCND-Nle-PITNDQKKLCSSQCQSVRa,
SEQ ID NO: 68:
NSECLSLCND-Nle-PITNDQKKLCSSSCQSVRa,
SEQ ID NO: 69:
QSECLSLCND-Nle-PITNDQKKLCSN-Dab-CQSVRa,
SEQ ID NO: 70:
QSECLSLCND-Nle-PITNDQKKLCSS-Dab-CQSVRa,
SEQ ID NO: 71:
QSECLSLCND-Nle-PITNDQKKLCSSNCQSVRa,
SEQ ID NO: 72:
QSECLSLCND-Nle-PITNDQKKLCSSQCQSVRa,
SEQ ID NO: 73:
QSECLSLCND-Nle-PITNDQKKLCSSSCQSVRa,
SEQ ID NO: 74:
SSECLSLCND-Nle-PITNDQKKLCSN-Dab-CQSVRa,
SEQ ID NO: 75:
SSECLSLCND-Nle-PITNDQKKLCSS-Dab-CQSVRa,
SEQ ID NO: 76:
SSECLSLCND-Nle-PITNDQKKLCSSNCQSVRa,
SEQ ID NO: 77:
SSECLSLCND-Nle-PITNDQKKLCSSQCQSVRa,
SEQ ID NO: 78:
SSECLSLCND-Nle-PITNDQKKLCSSSCQSVRa,
SEQ ID NO: 79:
GSECLSLCND-Nle-PITNDQKKLCSN-Dab-CQSVRa,
SEQ ID NO: 80:
GSECLSLCND-Nle-PITNDQKKLCSS-Dab-CQSVRa,
SEQ ID NO: 81:
GSECLSLCND-Nle-PITNDQKKLCSSNCQSVRa,
SEQ ID NO: 82:
GSECLSLCND-Nle-PITNDQKKLCSSQCQSVRa,
or
SEQ ID NO: 83:
GSECLSLCND-Nle-PITNDQKKLCSSSCQSVRa.

In another very preferred embodiment, said amino acid sequence (I) is a sequence selected from any one of SEQ ID NO: 45-88, preferably SEQ ID NO: 45-83, more preferably SEQ ID NO: 45-64, again more preferably SEQ ID NO: 45-51. In a most preferred embodiment, said amino acid sequence (I) is SEQ ID NO: 45 or 85.

In a certain embodiment, said amino acid sequence (I) of the cyclic peptide of the invention comprises (i) an N-terminus selected from a free amino group or an acetylated N-terminus, and/or (ii) a C-terminus selected from a free carboxyl group or an amidated C-terminus.

Preferred cyclic peptides are disclosed in the PCT application WO 2018/229156 A, the whole disclosure of which is incorporated by reference herein.

As explained further below, the particle of the invention may be used to provide boosting and/or priming vaccination against RSV, e.g. to provide priming vaccination by subcutaneous route following by boosting vaccination by epicutaneous route. Said particle preferably comprises a variant of an RSV-F protein as antigen; said variant being a cyclic peptide comprising an amino acid sequence (I) comprising, preferably consisting of SEQ ID NO: 44. In a particular embodiment, the amino acid sequence (I) comprises, preferably consists of a sequence selected from any one of SEQ ID NO: 45-88, preferably SEQ ID NO: 45-83, more preferably SEQ ID NO: 45-64, again more preferably SEQ ID NO: 45-51. In a most preferred embodiment, said amino acid sequence (I) is SEQ ID NO: 45 or 85. In another embodiment, said particle comprises a fragment of an RSV-F protein, preferably of SEQ ID NO:1. In a more preferred embodiment, said fragment comprises, preferably consists of a sequence selected from SEQ ID NO: 2-5. In another embodiment, said fragment comprises, preferably consists of sequence SEQ ID NO: 5.

Linker

In another embodiment, said RSV antigen, namely said RSV-F protein, fragment or variant thereof, especially the cyclic peptide may further comprise a linker. In order to form the conjugate of the invention, one or more antigens of the invention may be conjugated to the peptide moiety of the lipopeptide building block, either directly or through a linker, either via the N- or C-terminus of the antigen of the invention. Said antigens of the invention are connected either to the N- or to the C-terminal of the peptide moiety or optionally to one or more amino acid side chains of the peptide moiety. Alternatively, the antigen of the invention is conjugated to the peptide moiety of the lipopeptide building block through a side chain residue of the antigen of the invention, such as a terminal or internal aspartic acid, glutamic acid, lysine, ornithine or cysteine side chain.

Said linker is preferably attached to said antigen of the invention, preferably to the cyclic peptide of the invention, and wherein said linker comprises (i) at least one attachment moiety, (ii) at least one spacer moiety, (iii) at least one linking moiety, or (iv) any combination of (i), (ii) and (iii).

In another preferred embodiment, said at least one attachment moiety comprises or preferably consists of —O—NH₂, —O—NH— (an aminooxy moiety), —C(O)—CH₂—O—NH₂, —C(O)—CH₂—O—NH— (aminooxy acetyl moiety), —NH—NH₂, —NH—NH— (hydrazine moiety), -E(O)—NH—NH₂, or -E(O)—NH—NH— (hydrazide moiety), wherein E is C, S(O) or P. In a further preferred embodiment, said attachment moiety comprises or preferably consists of an —O—NH₂, —O—NH— (an aminooxy moiety), —C(O)—CH₂—O—NH₂, —C(O)—CH₂—O—NH— (aminooxy acetyl moiety), —NH—NH₂, —NH—NH— (hydrazine moiety), or (—C(O)—NH—NH₂, —C(O)—NH—NH-(carbohydrazide moiety). In another further preferred embodiment, said attachment moiety comprises or preferably consists of —O—NH₂ or —O—NH— (an aminooxy moiety).

In another preferred embodiment, said at least one spacer moiety comprises or preferably consists of NH₂—CH₂—CH₂—(O—CH₂—CH₂)ₙ—C(O)— or —NH—CH₂—CH₂—(O—CH₂—CH₂)ₙ—C(O)—, wherein n is an integer of 1 to 45, preferably 2 to 20, more preferably 6 to 8; or NH₂—(CH₂)ₘ—C(O)— or —NH—(CH₂)ₘ—C(O)—, wherein m is an integer of 2 to 45, preferably 2 to 20, more preferably 2 to 6.

In one embodiment of the invention, said linking moiety is capable of cross-linking the cyclic peptide with a second peptide and comprises or consists of an aldehyde moiety, such as a glutaraldehyde moiety, octanedialdehyde moiety, dialdehyde moiety, succinaldehyde moiety; carbodiimide moiety, such as 1-ethyl-3-(3-dimethylaminopropyl)carbodiimide hydrochloride moiety; glyoxol moiety; N-hydroxy-sulphosuccinimidyl moiety, such as N-hydroxy-sulphosuccinimidyl moiety; a cationic linking moiety; polyethyleneglycol moiety; benzoyl benzoic acid moiety. Further suitable linking moieties are listed in the Pierce Catalog and Handbook, Pierce Chemical Company, Rockford (1997); Bioconjugate Techniques, Greg T. Hermanson, Pierce Biotechnology, Thermo Fisher Scientific, Rockford (2013); and are described in EP 1321466 A1, DE 19821859 A1, U.S. Pat. Nos. 6,875,737, 5,456,911, 5,612,036, 5,965,532, WO 2001004135, WO 2001070685, US 20140302001 A1, U.S. Pat. No. 6,800,728, US 20140171619 A1, U.S. Pat. No. 8,168,190, WO 2012/166594 A1 and WO 2015/082501.

In a preferred embodiment, the linker is attached to the amino acid sequence (I) typically and preferably via an amide bond to the N-terminus of said amino acid sequence (I) or to a free amino group of a side chain of an amino acid of said amino acid sequence (I), preferably to the N-terminus of said amino acid sequence (I). In a preferred embodiment, said linking moiety is capable of cross-linking said cyclic peptide with a thiol group of a second peptide. In a preferred embodiment, said linking moiety comprises a maleimide moiety.

In a preferred embodiment, said linker is attached to an amino group included in said amino acid sequence (I), wherein preferably said linker is attached to a free amino group of (i) the N-terminus of said amino acid sequence (I), or (ii) a side chain of an amino acid of said amino acid sequence (I). Preferably, the linker is attached to said amino group included in said amino acid sequence (I) by an amide bond. Said side chain is preferably of the amino acid lysine. In a preferred embodiment, X24 is lysine and said linker is attached to the free amino group of the side chain of X24.

In a very preferred embodiment, said linker is selected from the following formulas:

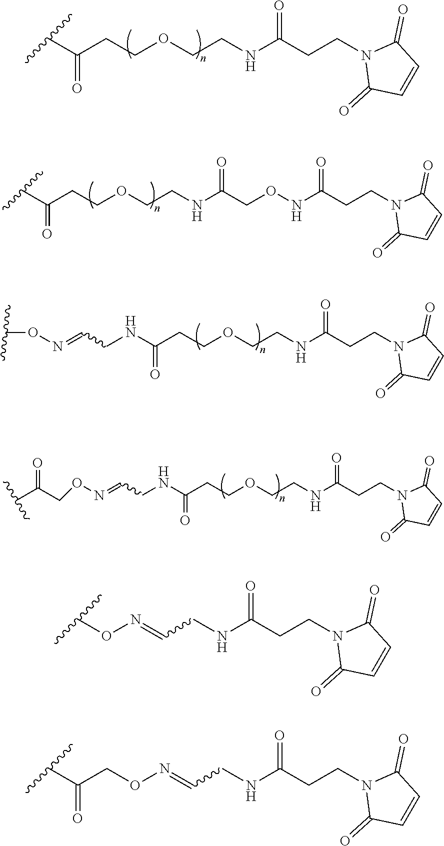

wherein n is an integer of 1 to 45, preferably 6 to 8, and the terminal wavy line indicates the attachment site to said amino acid sequence (I). Further very preferred, said n is 6. Within the chemical formulas presented herein said double bond or said oxime moiety, is typically and preferably represented by a wavy line. The maleimide group may enable the chemical coupling of the RSV antigen with the lipopeptide building block by reaction with a thiol group present in the peptide moiety of said lipopeptide building block.

Most preferably, said linker is

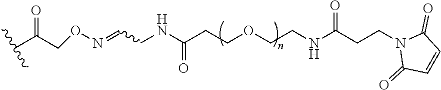

wherein n is an integer of 1 to 45, preferably 6 to 8, more preferably 6, and the terminal wavy line indicates the attachment site to the RSV-antigen, e.g. the cyclic peptide comprising or consisting of said amino acid sequence (I).

In a further very preferred embodiment, said cyclic peptide comprises, preferably is, a formula selected from any one of following formulas:

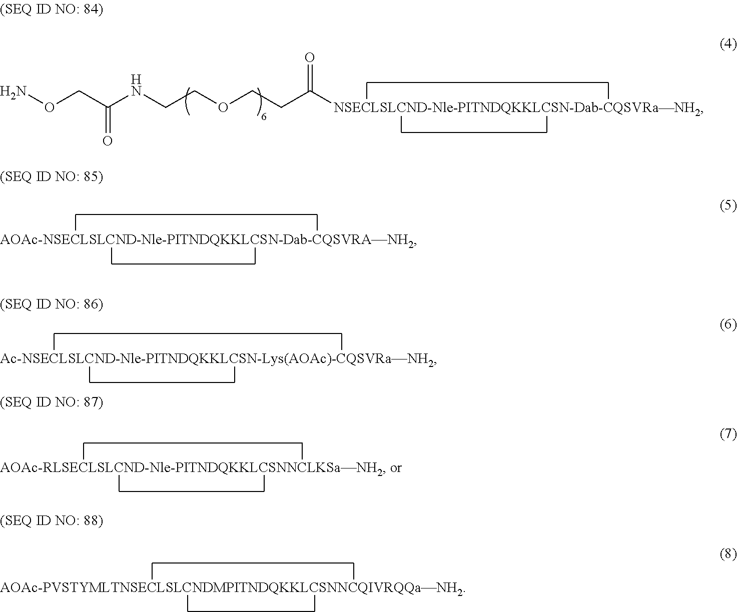

In a further very preferred embodiment, said cyclic peptide comprises, preferably is, a formula selected from any one of formulas (4) (SEQ ID NO: 84), formula (5) (SEQ ID NO: 85), formula (6) (SEQ ID NO: 86), formula (7) (SEQ ID NO: 87), formula (8) (SEQ ID NO: 88),

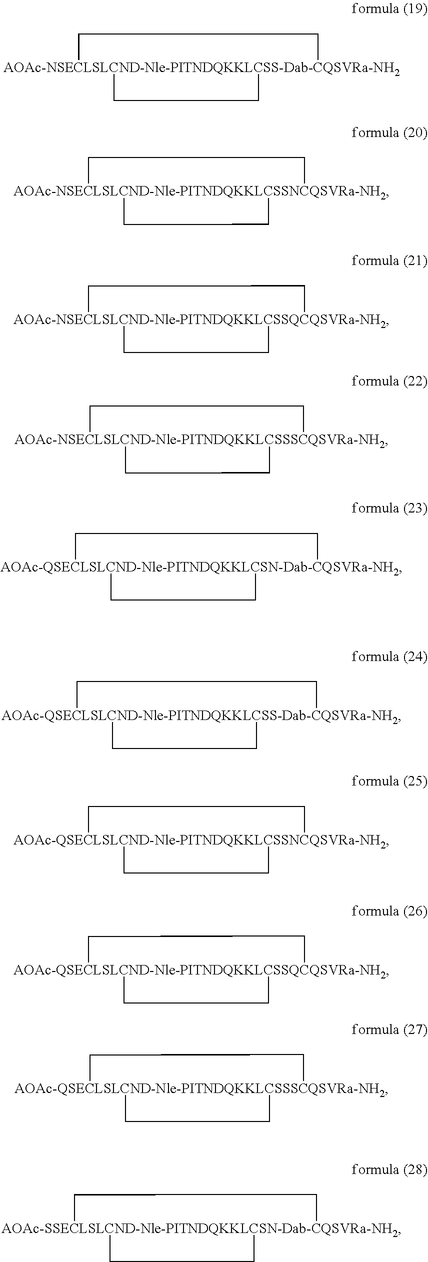

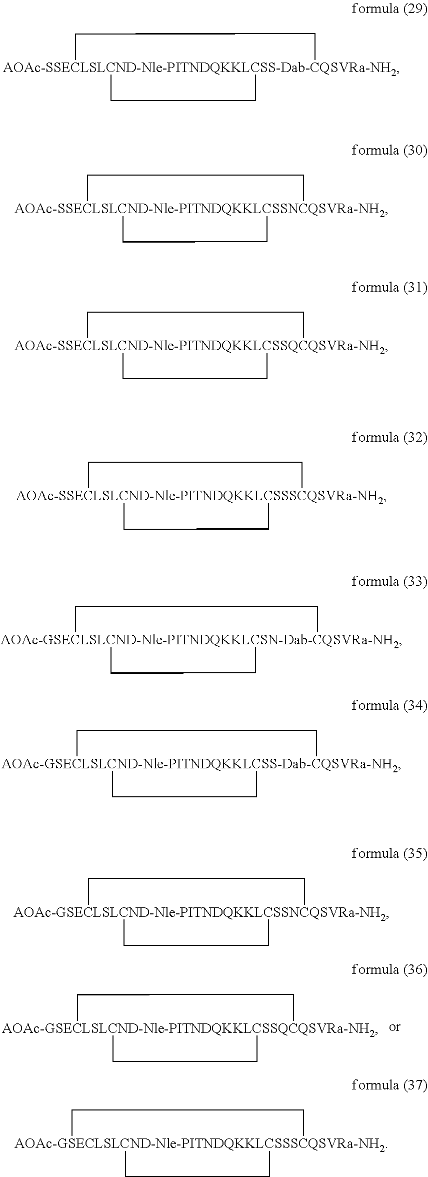

In a most preferred embodiment, said cyclic peptide comprises formula (85).

For instance, the cyclic peptide comprises the peptide of formula (85) which is attached to a linker. In a preferred embodiment, the cyclic peptide-linker moiety is of formula V-306pL as described below, wherein the wavy line indicates the attachment site to the peptide chain of a lipopeptide building block of the conjugate:

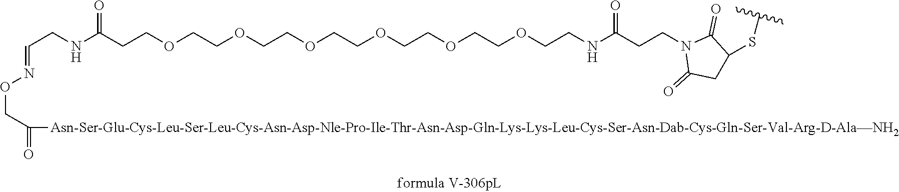

Conjugation procedures that may be used to attach the cyclic peptide to the lipopeptide building block are well known to those skilled in the art (see for example Hermanson, G. T, Bioconjugate Techniques, 2nd edition, Academic Press, 2008). Any method used for conjugating peptides or other antigens to an antigen delivery system, such as carrier protein, polymer, dendrimer, nanoparticle or virus-like particle, can be used to conjugate said cyclic peptide to said lipopeptide building block.

All embodiments and preferred and very preferred embodiments of the inventive cyclic peptide described herein are applicable to all aspects of the present invention, especially to the aspect of the conjugate comprising the RSV-F protein, a fragment or variant thereof, especially the cyclic peptide, and to the aspect of the lipopeptide building block of the invention, even though not all embodiments and preferred and very preferred embodiments of the cyclic peptide are again repeated. Also all embodiments and preferred and very preferred embodiments of the lipid building block, the conjugate, and all of its components including antigens and linker etc. described herein are applicable to all aspects of the present invention, even though not all embodiments and preferred and very preferred embodiments are not necessarily again repeated and reiterated.

As described herein, the terms “vaccination” and “immunization” designate the sequential administration of one or more antigens to a subject, to produce and/or enhance an immune response against the antigen(s), preferably to protect the infant or the foetus. Preferably, the vaccination leads to a long lasting and effective protection against a given pathogen.

The vaccination may be useful to treat or prevent (e.g. delay, reduce, avoid, eliminate) a disorder caused by the pathogen or resulting from an infection by the pathogen.

In the context of vaccination, the administration of the antigen can be sequential and can typically include a priming immunization followed by one or several boosting immunizations. As used herein, the term “vaccination” encompasses (i) priming vaccination wherein the antigen is administered in a naïve subject in order to induce an immune response in said subject, (ii) boosting vaccination wherein the antigen is administered to boost, namely amplify a pre-existing immune response, in the subject as well as (iii) priming/boosting vaccination wherein the antigen is first administered to induce an immune response, and then administered again to boost the resulting immune response.

Thus, unless stated otherwise, the term “vaccination” encompasses prime vaccination, boost vaccination as well as prime vaccination followed by boosting vaccination, wherein the latter is more preferred.

Preferably, said “subject” is a mammal, more preferably a human. Typically said human is selected from the group of infants, children, adults of any gender including the elderly, women of childbearing age, a pregnant women and women under lactation. The invention also allows protection of infants, e.g. children of less than 1 year, preferably below 6 months old, more typically children below 5 months old. In a preferred embodiment, the subject is a woman of childbearing age or a pregnant woman. The inventors have shown that the epicutaneous prime and boost vaccinations with the particle of the invention lead to RSV-specific immune responses. They have further shown that a prime vaccination with a subcutaneous injection of the particles of the invention, followed by an epicutaneous boost vaccination with said particles leads to the generation of RSV-neutralizing antibodies

As used herein, a “neutralizing antibody” or a “RSV-neutralizing antibody” refers to an antibody that is binding to RSV-F, a variant of a fragment thereof and results in inhibition of at least one biological activity of RSV-F. For example, a neutralizing antibody may aid in blocking the fusion of RSV to a host cell, or prevent syncytia formation, or prevent the primary disease caused by RSV. Alternatively, an antibody of the invention may demonstrate the ability to ameliorate at least one symptom of the RSV infection. This inhibition of the biological activity of RSV-F protein can be assessed by measuring one or more indicators of RSV-F biological activity by one or more of several standard in vitro assays (such as a neutralization assay) or in vivo assays known in the art.

Preferably, in the context of the present invention, vaccination refers to boost vaccination, so as to amplify a pre-existing immune response against RSV in a subject. The pre-existing immune response in the subject may result from a conventional prime vaccination or may result from a previous, natural, exposure to the RSV.

In a specific embodiment, the particles of the invention are used for epicutaneous boost vaccination after a conventional vaccination against RSV. In this embodiment, the invention thus resides in the use of a particle of the invention comprising an RSV-F protein, a variant or a fragment thereof, preferably a SVLP of the invention, to stimulate an existing immune response against RSV in a subject having received a conventional vaccine, said particle being administered to the subject by epicutaneous route, e.g. by skin application(s).

As used therein, “conventional” administration or vaccination designates the parenteral, oral or nasal administration or vaccination. Parenteral administration can be performed by injection (e.g., intramuscular, intradermal, intravenous or subcutaneous), puncture, and/or transdermal administration. A preferred parenteral administration route is through injection, more preferably through subcutaneous injection.

In some embodiment, the conventional vaccine refers to a previous administration of the particle of the invention, preferably the SVLP of the invention, by subcutaneous route to the subject.

In a particular embodiment, the particles of the invention, preferably the SVLP of the invention, are used for both priming an immune response by subcutaneous injection and boosting the resulting response by epicutaneous route in a subject in need thereof.

In another embodiment, the particles of the invention, preferably the SVLP of the invention, are used to provide priming and/or boosting vaccination against RSV by epicutaneous route in a subject in need thereof.

In a further embodiment, the particles of the invention, preferably the SVLP of the invention, are used for providing boosting vaccination by epicutaneous route in a subject having a pre-existing immunity against RSV.

The inventors have shown that particles exposing RSV-F applied epicutaneously effectively leads to the generation of an immune response in vivo. Such a response has the properties required to provide effective protection against RSV, and which protection can be transferred to a foetus or newborn to ensure very early protection. More precisely, particles exposing RSV-F applied epicutaneously effectively lead to the generation of neutralizing antibodies directed against RSV-F protein.

Maternal vaccination appears to be the most efficient and safe strategy to prevent in neonate and infants. In a specific embodiment, the epicutaneous vaccination can thus be performed directly on a pregnant female to vaccinate the infant. In one embodiment, the invention thus allows to passively vaccinate children or infants via the transfer of protective antibodies across the placenta, including neutralizing antibodies directed against RSV-F protein. In an alternative embodiment, the invention relies on the transfer of protective antibodies through breast milk.

Thus, in a second aspect, the invention relates to a particle comprising an RSV-F protein for use in a method for vaccinating an infant against RSV by maternal epicutaneous vaccination with said particle.

In other words, the invention also relates to a particle comprising an RSV-F protein for use in a method for inducing passive immunity against RSV in a fetus or a breast-feeding infant by maternal epicutaneous vaccination with said particle.

Indeed, in a particular embodiment, the invention relates to epicutaneous maternal vaccination against RSV. Maternal vaccination designates vaccination of a female during pregnancy or lactation, leading to effective protection of the foetus or infant through immunity transfer. Such a maternal epicutaneous vaccination strategy effectively protects the treated female as well as the foetus or infant and, more particularly,
(i) delays the acquisition of primary RSV infection until the infant airways are larger and their immune system is mature, and
(ii) reduces the severity of disease if infection does occur afterbirth. Such a treatment process confers remarkable advantage and allows early and effective, although non-invasive, protection of new-borns.

Thus, maternal epicutaneous vaccination provides passive immunity to the foetus and to the breast-feeding infant by antibody transfer via placenta or breast-feeding respectively.

Preferably, maternal vaccination comprises vaccination of the female during pregnancy and/or lactation, i.e. during the breastfeeding period. Thus, in a preferred embodiment, the particle of the invention is applied epicutaneously to a pregnant female, e.g. during the second and third quarter of the pregnancy, preferably during the second quarter. Such treatment allows generation of an effective immune response by the female, including the generation of neutralizing antibodies directed against RSV-F protein and the passive transmission thereof to the foetus.

In some embodiments, the woman has a pre-existing immunity against RSV, e.g. due to previous natural infection with the virus or due to a previous vaccination against RSV, e.g. by means of a vaccine administered by subcutaneous route.

Indeed, most adults have experienced several RSV infections during their life, specific immunity is short lived, leading to a high heterogenicity between individuals in terms of protective immunity. In this regard, the particle of the invention, in particular the SLVP may be epicutaneously administered to boost a pre-existing immune response against RSV in a subject, in particular an adult, e.g. by recalling memory B-cells induced by a previous infection with RSV.

Accordingly, in a particular embodiment, the invention relates to the use of a particle of the invention, in particular a SVLP of the invention, for boosting pre-existing immunity against RSV in a woman of child-bearing age and/or promoting passive immunity against RSV to her fetus and/or neonate, wherein the woman is epicutaneously administered with the particle of the invention before pregnancy.

Preferably, the epicutaneous administration of the SVLP is repeated at least once (e.g. twice) during pregnancy and/or lactation.

In another particular embodiment, the invention relates to the use of a particle of the invention, in particular a SVLP of the invention, for promoting immunity against RSV in a woman of child-bearing age and/or promoting passive immunity against RSV in her fetus and/or neonate, wherein
(i) the woman is subcutaneously administered with the particle of the invention before pregnancy and
(ii) the woman is epicutaneously administered with the particle of the invention during pregnancy and/or lactation.

In step (ii), the administration of the particle of the invention may be performed by using a skin patch as fully described further below.

Step (ii) enables to boost the immune response against RSV induced in step (i). In a particular embodiment, step (ii) is repeated at least once, e.g. one or two times, typically no earlier than one month, typically within two or three months from the first epicutaneous administration.

In a preferred embodiment, the particle of the invention is a nanoparticle. In another more preferred embodiment, the particle of the invention is a virus like particle (VLP). As used herein, the term “virus-like particle” (VLP) refers to a non-replicating, multicomponent structure composed of one or more viral proteins or virally-derived peptides or polypeptides, such as, but not limited to capsid, coat, shell, surface and/or envelope proteins, or variant polypeptides derived from these proteins.

In a more preferred embodiment, said particle is a synthetic virus-like-particle (SVLP). Preferably, said SVLP comprises, preferably consists, of conjugates, wherein each conjugate comprises:
a) a peptide chain comprising a coiled coil-domain, linked covalently to
b) a lipid moiety comprising three of preferably two long hydrocarbyl chains, and
c) an RSV-F protein, a variant, or a fragment thereof.

An SVLP of the invention comprises a (i) lipopeptide building block and (ii) an RSV-F protein, a variant, or a fragment thereof, wherein said lipopeptide building bock comprises (a) a peptide chain comprising a coiled coil-domain, linked covalently to (b) a lipid moiety comprising three of preferably two long hydrocarbyl chains. SVLPs are disclosed in the PCT application WO 2008/068017 and Ghasparian A et al., ChemBioChem 2011, 12, 100-109, the disclosure of which is incorporated by reference herein. Conjugates as herein defined will self-assemble to helical lipopeptide bundles (HLB) and further to synthetic virus-like particles (SVLP)(FIG. 8C). The self-assembly process in aqueous solution includes the rapid oligomerization of the coiled-coil domains of the conjugates to form a parallel coiled-coil bundle of alpha-helices of defined oligomerization state; referred to as a HLB. As a result the lipid moieties attached to the peptide chains within each HLB also aggregate at one end of the bundle. Furthermore, multiple copies of the RSV-F protein, a variant or fragment thereof are to be presented on the surface of the HLB.

The HLB can self-assemble, resulting in the formation of SVLP. The process is driven by the self-association of the lipid tails attached to each building block, which then occupy the central lipid core of the SVLP. In this way, the peptide chains in each helical bundle are oriented outwards, towards the bulk solvent. The size and composition of the conjugate thus determines the final size and shape of the SVLPs, the diameters of which are typically in the nanometer range (10-30 nm).

In some embodiments, the SVLP have a diameter of less than 100 nm, preferably of less than 50 nm. Preferably, said SVLP have a diameter comprised between about 15 nm and about 20 nm, more preferably of about 20 nm. In another more preferred embodiment, said SVLP have a diameter between 10 nm and 40 nm, e.g. between about 15 nm and about 30 nm, more preferably of about 20 nm to about 30 nm, again more preferably of about 25 nm to about 30 nm. Preferably, the diameter is measured via Dynamic Light Scattering (DLS) and transmission electron microscopy as described herein (FIG. 10, Example 2). It is noteworthy that the SVLP according to the invention are composed of protein and lipid components, as found in real viruses, have physical dimensions resembling those of some small viruses, have a lipid core and an external protein/peptide-based outer surface, but are totally of synthetic origin, i.e. are produced by chemical synthesis starting from conjugates without using cell-based methods. Thus, all their components are produced by chemical synthesis, hence avoiding the use of materials that must be made using biological methods.

Having multiple copies of the RSV-F protein, a variant or a fragment thereof on the surface of the SVLP enhances B-cell receptor affinity to the antigen through an avidity effect and facilitates uptake and presentation of the particle or its components by immunocompetent cells. The HLBs and SVLPs may, therefore, be viewed as macromolecular carriers, or delivery vehicles for antigens, for the purpose of raising efficient immune responses against said antigen in an animal.

The conjugates of the invention are designed in such a way that the coiled-coil domain in the lipopeptide will assemble to a defined helical bundle (e.g. dimeric, trimeric, tetrameric, pentameric, hexameric or heptameric bundle of helices, preferably a trimeric bundle of helices). This association leads to the formation of HLBs. The resulting helical lipopeptide bundles (HLBs) will self-assemble into a synthetic virus-like particle (SVLP) (macromolecular assembly) with dimensions on the nanometer scale.

Preferably, said coiled coil peptide chain segments of said peptide moieties of said conjugate form in said bundle a left-handed alpha-helical coiled coil, wherein the coiled coil peptide chain segments have a parallel orientation in said coiled coil. Preferably, said SVLP of the invention has a mean hydrodynamic radius (Rh) of 10-20, e.g. ca. 13 nm, measured via DSL preferably in PBS as described in the Examples. Preferably, said SVLP of the invention are monodisperse particles. For instance, said SVLP can show a polydispersity index of 0-0.1, more 0.02-0.08, again more preferably 0.04-0.06, e.g. about 0.05. In a preferred embodiment, the SVLP of the invention comprises about 30-150, more preferably 60-90 copies of the conjugate, e.g. the conjugates of formula (38). Preferably, the lipid chains of the conjugates are buried in the core of the SVLP, and the RSV-F protein, a variant or a fragment thereof is exposed in the SVLP surface.

In a preferred embodiment, SVLP of the invention are formed by aggregation of trimeric bundles of conjugates, i.e. each bundle consists of three conjugates. Typically, said SVLP has a diameter of ca. 25-30 nm. Said SVLP typically comprises about 60-90 copies of the conjugate. Said SVLP may show a polydispersity index of about 0.05. Preferably the conjugate of the SVLP is of formula (38).

Peptide Chain (Peptide Moiety)

The lipopeptide building block comprises a peptide chain, also called peptide moiety herein. Said peptide chain (PC) comprises coiled-coil domains. Such coiled-coil domains will associate into a defined helical bundle, e.g. into a dimeric, trimeric, tetrameric, pentameric, hexameric or heptameric bundle.

In a preferred embodiment, said peptide moiety has a length of 12 to 200 amino acids, more preferably of 21 to 120 amino acids, again more preferably of 21 to 80 amino acids, again more preferably of 21 to 70 amino acids again more preferably of 21 to 60 amino acids again more preferably of 21 to 50 amino acids, again more preferably said peptide moiety has a length of 28 to 48 amino acids. Preferred peptide moieties are non-human sequences to avoid the risk of autoimmune disorders when applied in the vaccination of humans.

Coiled-coil domains contain multiple tandem repeat motifs, which as self-standing lipid-free peptides possess the property of self-assembly into a parallel coiled-coil helical bundle. The peptide chain (PC) must multimerize to form a parallel coiled-coil helical bundle of defined oligomerization state (e.g. dimer, trimer, tetramer, pentamer, hexamer or heptamer, in particular dimer, trimer, tetramer or pentamer). Therefore, the coiled-coil domains are designed by careful selection of appropriate amino acid sequences that form a thermodynamically stable, alpha-helical, parallel bundle of helices by spontaneous self-association.

As used herein, the term “coiled coil peptide chain segment” is a sequence of a peptide chain capable of forming a coiled coil (super coil). A coiled coil is a peptide structure in which at least two coiled coil peptide chain segments, each having preferably an alpha helical secondary structure, are associated into a bundle. In one embodiment, said peptide moiety comprises more than one coiled coil peptide chain segment, wherein preferably said coiled coil peptide chain segments form a bundle. Preferably said bundle is monomeric, i.e. said coiled coil peptide chain segments are included in one peptide chain.

Coiled coil peptide chain segments of the invention contain multiple repeat units, preferably consecutively linked to each other. The repeat units of the coiled coil peptide chain segment may be identical or may be different, e.g. may contain at least one discontinuity, such as an insertion, deletion or exchange of at least one, preferably exactly 1, 2, 3 or 4 amino acids within the repeat unit. In a preferred embodiment, said coiled coil peptide chain segment of said peptide moiety consists of 2 to 10 repeat units, preferably 3 to 8 repeat units, more preferably four repeat units. The upper number of repeat units in the peptide moiety influences the stability of the coiled coil. It is limited mainly by the feasibility of chemical synthesis of long peptides, but sequences containing more than three heptad repeats (e.g. four, five, six, seven, eight or ten repeat units) are preferred.

Repeat units of coiled coil peptide chain segments have a sequence with a certain number of amino acids, wherein the positions of the amino acids are traditionally labelled as lowercase letters. Design rules are discussed in more detail, for example, in Woolfson, D. N., Adv. Prot. Chem. 2005, 70, 79-112. A coiled-coil domain includes peptides based on canonical tandem heptad sequence repeats that form right-handed amphipathic α-helices, which then assemble to form helical bundles with left-handed supercoils. Canonical coiled-coils occur widely in naturally occurring biologically active peptides and proteins, and have also been designed de novo. A set of rules has been elucidated for designing coiled-coil peptides that adopt helical bundles of defined oligomerization state, topology and stability (e.g. dimer, trimer, tetramer, pentamer, hexamer or heptamer). These rules allow designers to build a peptide sequence compatible with a given target structure. Most important, the sequences of canonical coiled-coil peptides contain a characteristic seven-residue motif. The positions within one heptad motif are traditionally denoted abcdefg, with mostly (but not exclusively) hydrophobic residues occurring at sites a and d and generally polar, helix-favoring residues elsewhere. Tandem heptad motifs along a peptide chain have an average separation between the a and d residues that allows them to fall on one face of the alpha-helix. When two or more helices pack together into a coiled-coil bundle the hydrophobic faces of the helices associate and wrap around each other in order to maximize contacts between hydrophobic surfaces. The type of residue that may occur at each position within a heptad repeat will influence the stability and oligomerization state of the helical bundle. In general, mostly hydrophobic residues (Ala, Ile, Leu, Met, Val), or aromatic hydrophobic side chains (Phe, Trp and Tyr), are used at the a and d sites. The remaining b, c, e, f and g sites tend to be more permissive than the a and d sites, though polar and helix-favoring residues (Ala, Glu, Lys and Gln) are favored. The choice of residues at the a and d sites can influence the oligomerization state of the coiled coil (i.e. dimer vs. trimer). Thus, dimers are favored when non-β-branched residues (e.g. Leu) occur at the d positions; at these sites β-branched residues (Val and Leu) disfavor dimers. On the other hand, in dimers β-branched residues (Ile, Val) are preferred at the a sites. Another rule is that a=d=Ile or Leu favors trimers, which is useful in designing coiled coils that specifically form parallel trimers.

In a certain embodiment of the invention, said repeat unit of the coiled coil peptide chain segments consists of 7 to 15 amino acids, preferably 7 to 11 amino acids. More preferably said repeat unit is a heptad motif consisting of 7 amino acids. In a preferred embodiment, said heptad motif includes amino acids having hydrophobic residues at positions a and d, and preferably polar, helix-favoring residues at the other residues. In a further preferred embodiment, said heptad motifs has seven amino acid positions designated with letters a b c d e f g, and wherein positions a and d in each heptad motif comprise independently of each other: (a) an alpha-amino acid with a hydrophobic side chain and/or an aromatic or hetero-aromatic side chain; (b) in zero, one or two of all the a and d positions an amino acid with a polar non-charged residue, and in zero or one of all the a and d positions (i) an amino acid with a polar cationic residue or an acetylated derivative thereof, or (ii) an amino acid with a polar anionic residue, or (iii) glycine. Preferably, said alpha-amino acid with a hydrophobic side chain is alanine, isoleucine, leucine, methionine and valine; alpha-amino acids with aromatic or hetero-aromatic residue are phenylalanine, tyrosine, tryptophan and histidine; alpha-amino acids with polar non-charged residue are asparagine, cysteine, glutamine, serine and threonine; alpha-amino acids with polar cationic residue are arginine, lysine and histidine; and alpha-amino acids with polar anionic residue are aspartic acid and glutamic acid. The heptad motif codes for amphipathic α-helices that oligomerize through their hydrophobic faces. The coiled-coil domain includes at least three tandem heptad repeat motifs. The upper number of heptad repeats in each chain will influence the stability of the helical bundle. It is limited mainly by the feasibility of chemical synthesis of long peptides, but sequences containing more than three heptad repeats (e.g. four, five, six, seven and eight heptad repeats) are preferred.

Examples of coiled-coil peptide sequences occurring naturally in viral coat proteins are coiled-coil motifs forming trimeric helical bundles in the gp41 coat protein of HIV-1 and the F protein of RSV. The preferred coiled-coil peptides should contain between 3-8 tandemly linked heptad motifs. The heptad motifs within the coiled coil may have identical sequences, or they may each have different sequences. In all cases, the seven positions of the seven amino acid residues within one heptad motif are designated with letters: a b c d e f g. The coiled coil peptide, therefore, comprises an amino acid sequence having the positions (abcdefg)₃₋₈.

Preferred are coiled-coil peptide sequences containing between 3-8 tandemly linked heptad motifs, wherein positions a and d in each heptad motif (abcdefg) contain alpha-amino acids belonging to the Group 1 and/or to the Group 2 as defined hereinbelow. In addition, not more than two of all the a and d positions may be occupied by any amino acid residue belonging to the Group 3, and not more than one of all the a and d positions may be occupied by any amino acid residue belonging to the Group 4 or Group 5 or by glycine. In addition, in positions b, c, e, f and g, alpha-amino acids belonging to the Groups 3, 4 and 5 are preferred, but amino acids belonging to the Groups 1 and 2 are allowed, with the addition that not more than one of these positions within any one heptad motif may be glycine, but none may be proline.

Preferably, group 1 comprises alpha-amino acid residues with small to medium sized hydrophobic side chains R¹.

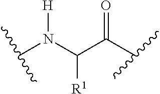

A hydrophobic residue R¹ refers to an amino acid side chain that is uncharged at physiological pH and that is repelled by aqueous solution. These side chains generally do not contain hydrogen bond donor groups, such as primary and secondary amides, primary and secondary amines and the corresponding protonated salts thereof, thiols, alcohols, ureas or thioureas. However, they may contain hydrogen bond acceptor groups such as ethers, thioethers, esters, tertiary amides, or tertiary amines. Genetically encoded amino acids in this group include alanine, isoleucine, leucine, methionine and valine. Particular hydrophobic residues R¹ are lower alkyl, lower alkenyl, —(CH₂)a(CHR²)_{b}OR³, —(CH₂)ₐ(CHR²)_{b}SR³, —(CHR²)OR³, —(CH₂)ₐSR³, —(CH₂)ₐR⁴, or —CH(CF₃)₂; wherein R² is lower alkyl; R³ is lower alkyl; R⁴ is cyclohexyl, cyclopentyl, or cyclobutyl; a is 1 to 4; and b is 0 or 1. Preferred hydrophobic residues are mentioned in WO 2008/068017, e.g. in claim 6.

Preferably group 2 comprises amino acid residues with aromatic or heteroaromatic side chains R⁵.

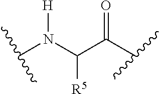

An aromatic amino acid residue refers to a hydrophobic amino acid having a side chain R⁵ containing at least one ring having a conjugated aromatic π(pi)-electron system. In addition it may contain additional hydrophobic groups such as lower alkyl, aryl or halogen, hydrogen bond donor groups such as primary and secondary amines, and the corresponding protonated salts thereof, primary and secondary amides, alcohols, and hydrogen bond acceptor groups such as ethers, thioethers, esters, tertiary amides or tertiary amines. Genetically encoded aromatic amino acids include phenylalanine and tyrosine. A heteroaromatic amino acid residue refers to a hydrophobic amino acid having a side chain R⁵ containing at least one ring having a conjugated aromatic pi-system incorporating at least one heteroatom such as O, S and N. In addition, such residues may contain hydrogen bond donor groups such as primary and secondary amides, primary and secondary amines and the corresponding protonated salts thereof, alcohols, and hydrogen bond acceptor groups such as ethers, thioethers, esters, tertiary amides or tertiary amines. Genetically encoded heteroaromatic amino acids include tryptophan and histidine. Particular aromatic or heteroaromatic side chains R⁵ are —(CH₂)ₐR⁶, —(CH₂)cOCH₂)dR⁶, —(CH₂)_{c}S(CH₂)_{d}R⁶, or —(CH₂)_{c}NR⁷(CH₂)_{d}R⁶; wherein R⁷ is H₁ lower alkyl, aryl, or aryl-lower alkyl; R⁶ is optionally substituted phenyl of formula —C₆R⁸R⁹R¹⁰R¹¹R¹² or an aryl- or hetero-aryl group of one of the formulae H1 to H14

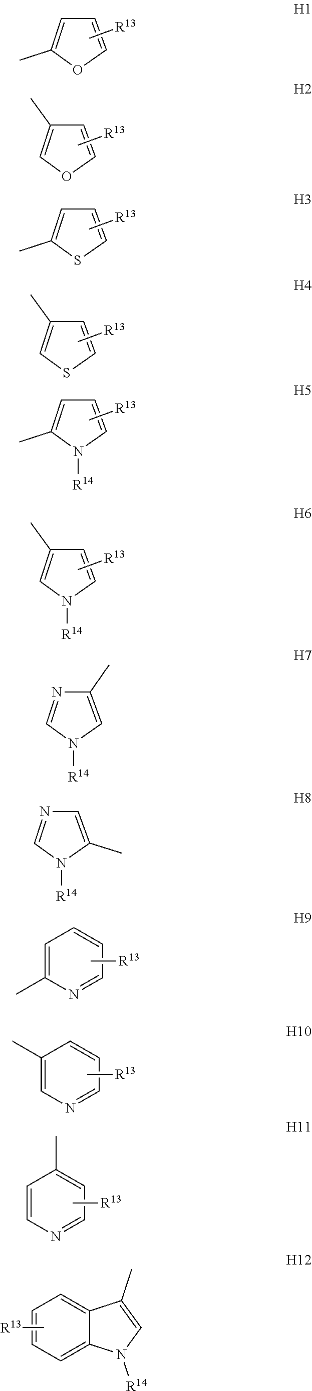

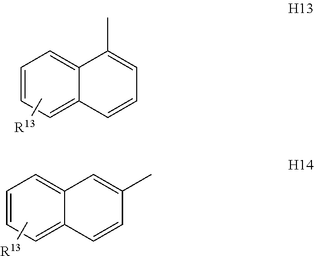

wherein each of R⁸, R⁹, R¹⁰, R¹¹ and R¹² is, independently of each other, H, F, Br₁Cl, I, NO₂, CF₃, NR⁷R¹⁴, N⁷COR¹⁴, lower alkyl, aryl, or OR⁷; R¹³ is H, Cl, Br, I, NO₂, lower alkyl, or aryl; R¹⁴ is H, lower alkyl, or aryl; a is 1 to 4; c is 1 or 2; and d is 0 to 4. Preferred aromatic or heteroaromatic side chains are mentioned in WO 2008/068017, e.g. in claim 6.

Preferably, group 3 comprises amino acids containing side chains with polar non-charged residues R¹⁵.

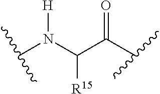

A polar non-charged residue R¹⁵ refers to a hydrophilic side chain that is uncharged at physiological pH, but that is not repelled by aqueous solutions. Such side chains typically contain hydrogen bond donor groups such as primary and secondary amides, primary and secondary amines, thiols, and alcohols. These groups can form hydrogen bond networks with water molecules. In addition, they may also contain hydrogen bond acceptor groups such as ethers, thioethers, esters, tertiary amides, or tertiary amines. Genetically encoded polar non-charged amino acids include asparagine, cysteine, glutamine, serine and threonine. Particular polar non-charged residues R¹⁵ are —(CH₂)_{d}(CHR¹⁶)_{b}OR¹⁷, —(CH₂)_{d}(CHR¹⁶)_{b}SR¹⁷, —(CH₂)ₐCONR¹⁷R¹⁸, or —(CH₂)ₐCOOR¹⁹; wherein R¹⁶ is lower alkyl, aryl, aryl-lower alkyl, —(CH₂)ₐOR¹⁷, —(CH₂)ₐNR¹⁷R¹⁸, —(CH₂)ₐNR¹⁷R¹⁸, or —(CH₂)ₐCOOR¹⁹; R¹⁷ and R¹⁸ are, independently of each other, H, lower alkyl, aryl, or aryl-lower alkyl, or R¹⁷ and R¹⁸ taken together are —(CH₂)ₑ—, —(CH₂)₂—O—(CH₂)₂—, or —(CH₂)₂—NR¹⁷—(CH₂)₂—; R¹⁹ is lower alkyl, aryl, or aryl-lower alkyl; and wherein a, b and d have the meaning as defined above and e is 2 to 6. Preferred polar non-charged residues are mentioned in WO 2008/068017, e.g. claim 6.

Preferably, group 4 comprises amino acids containing side chains with polar cationic residues and acylated derivatives thereof, such as acylamino-derived residues and urea-derived residues R²⁰.

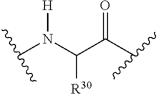

Polar cationic side chains R²⁰ refer to a basic side chain, which is protonated at physiological pH. Genetically encoded polar cationic amino acids include arginine, lysine and histidine. Citrulline is an example for a urea-derived amino acid residue. Particular polar cationic residues and acylated derivatives thereof R²⁰ are —(CH₂)ₐNR¹⁷R¹⁸, —(CH₂)ₐN═C(NR²¹R²²)NR¹⁷R¹⁸, —(CH₂)ₐNR²¹C(═NR²²)NR¹⁷R¹⁸, —(CH₂)ₐNR²¹COR¹⁹, or —(CH₂J₃NR²¹CONR¹⁷R¹⁸; wherein R²¹ is H or lower alkyl and R²² is H or lower alkyl; and R¹⁷, R¹⁸, R¹⁹ have the meaning as defined above and a is 1 to 4. Preferred polar cationic residue or acylated derivatives thereof are mentioned in WO 2008/068017, e.g. claim 6.

Preferably, group 5 comprises amino acids containing side chains with polar anionic residues R²³.

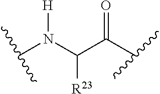

Polar anionic refers to an acidic side chain R²³, which is deprotonated at physiological pH. Genetically encoded polar anionic amino acids include aspartic acid and glutamic acid. A particular polar cationic residue R²³ is —(CH₂)ₐCOOH wherein a is 1 to 4. More preferred are coiled coil peptide chain segments containing between 3 to 8 tandemly linked heptad motifs, wherein each heptad motif (abcdefg) may have any one of the following sequences: (i) 1xx1xxx (referring respectively to the positions abcdefg); (ii) 1xx2xxx (referring respectively to the positions abcdefg); (iii) 2xx1xxx (referring respectively to the positions abcdefg); or (iv) 2xx2xxx (referring respectively to the positions abcdefg); wherein 1 is a genetically encoded amino acid from Group 1; 2 is a genetically encoded amino acid from Group 2; and wherein x is a genetically encoded amino acid from Groups 1, 2, 3, 4 or 5 or glycine. Preferred polar anionic residues are mentioned in WO 2008/068017, e.g. claim 6.

Lower alkyl is C₁₋₇-alkyl, preferably C₁₋₄-alkyl, in particular methyl, ethyl, n-propyl, iso-propyl, n-butyl or iso-butyl. Aryl has 5 to 10 carbon atoms and is preferably phenyl or naphthyl.

Preferred coiled coil peptide sequences are selected from the group consisting of sequences depicted in SEQ TD NO: 6, 7, 8, 9, 10, 11, 12, 13, 14, 15, 16, 17, 18, 19, 20, 21, 22, 23, 24, 25, 26, 27, 28, 29, 30 and variants thereof. Said sequences are depicted in the following Table 1:

| Origin | Sequence | SEQ ID NO |
|---|---|---|
| influenza virus | GSTQAAIDQINGKLNRVIEKTNEKFHQIE | SEQ ID NO: 6 |
| hemagglutinin | KEFSEVEGRIQDLEKYVEDTKCG | |
| human immuno- | SGIVQQQNNLLRAIEAQQHLLQLTVWGI | SEQ ID NO: 7 |
| deficiency virus | KQLQARILAVERYLGDCG | |
| bovine immuno- | GGERWQNVSYIAQTQDQFTHLFRNINNR | SEQ ID NO: 8 |
| deficiency virus | LNVLHHRVSYLEYVEEIRQKQVFFGCG | |
| feline immuno- | GGATHQETIEKVTEALKINNLRLVTLEH | SEQ ID NO: 9 |
| deficiency virus | QVLVIGLKVEAMEKFLYTAFAMQELGC | |
| | G | |
| equine infectious | GGNHTFEVENSTLNGMDLIERQIKILYA | SEQ ID NO: |
| anemia virus | MILQTHARVQLLKERQQVEETFNLIGCG | 10 |
| simian immuno- | GGAQSRTLLAGIVQQQQQLLDWKRQQE | SEQ ID NO: |
| deficiency virus | LLRLTVWGTKNLQTRVTAIEKYLKDQA | 11 |
| | GCG | |
| caprine arthritis | GGSYTKAAVQTLANATAAQQDVLEATY | SEQ ID NO: |
| encephalitis virus | AMVQHVAKGVRILEARVARVEAGCG | 12 |
| Visna virus | GGSLANATAAQQNVLEATYAMVQHVA | SEQ ID NO: |
| | KGIRILEARVARVEAIIDRMMVYQELDC | 13 |
| | G | |
| human parainfluenza- | GGEAKQARSDIEKLKEAIRDTNKAVQSV | SEQ ID NO: |
| 3 | QSSIGNLIVAIKSVQDYVNKEIVGCG | 14 |
| human parainfluenza- | GGEAREARKDIALIKDSIIKTHNSVELIQR | SEQ ID NO: |
| 1 | GIGEQIIALKTLQDFVNNEIRGCG | 15 |
| human parainfluenza- | GGKANANAAAINNLASSIQSTNKAVSDV | SEQ ID NO: |
| 2 | ITASRTIATAVQAIQDHINGAIVNGCG | 16 |
| human parainfluenza- | GGKAQENAKLILTLKKAATETNEAVRDL | SEQ ID NO: |
| 4a | ANSNKIWKMISAIQNQINTIIQGCG | 17 |
| human parainfluenza- | GGKAQENAQLILTLKKAAKETNDAVRD | SEQ ID NO: |
| 4b | LTKSNKIVARMISAIQNQINTIIQGCG | 18 |
| Measles virus | GGSMLNSQAIDNLRASLETTNQAIEAIRQ | SEQ ID NO: |
| | SGQEMILAVQGVQDYINNELIGCG | 19 |
| Mumps virus | GGAQTNARAIAAMKNSIQATNRAVFEV | SEQ ID NO: |
| | KEGTQQLAIAVQAIQDHINTIMNTQLNN | 20 |
| | MSCG | |
| Bovine respiratory | GGAVSKVLHLEGEVNKIKNALLSTNKA | SEQ ID NO: |
| syncytial virus | WSLSNGVSVLTSKVLDLKNYIDKEGCG | 21 |
| Ebola virus | GGANETTQALQLFLRATTELRTFSILNRK | SEQ ID NO: |
| | AIDFLLQRWGGTCHILGCG | 22 |
| Marburg virus | GGANQTAKSLELLLRVTTEERTFSLINRH | SEQ ID NO: |
| | AIDFLLTRWGGTCKVLGCG | 23 |
| Rous sarcoma virus | GGANLTTSLLGDLLDDVTSIRHAVLQNR | SEQ ID NO: |
| | AAIDFLLLAHGHGCG | 24 |
| Staphylothermus | GSIINETADDIVYRLTVIIDDR YESLKNLIT | SEQ ID NO: |
| marinus | LRADRLEMIINDNVSTILASIGCG | 25 |
| SARS coronavirus | GGNVLYENQKQIANQFNKAISQIQESLTT | SEQ ID NO: |
| | TSTALGKLQDWNQNAQALNTLVKQLSS | 26 |
| | NFGCG | |
| DUF16 domain of | GGTKTEFKEFQTWMESFAVQNQNIDAQ | SEQ ID NO: |
| MPN010 from | GEQIKELQVEQKAQGKTLQLILEALQGIN | 27 |
| Mycoplasma | KRLDNLESCG | |
| pneumoniae | | |
| heptameric coiled | GGKVKQLADAVEELASANYHLANAVAR | SEQ ID NO: |
| coil | LAKAVGERGCG | 28 |
| trimeric coiled | GGIEKKIEAIEKKIEAIEKKIEAIEKKIEAIE | SEQ ID NO: |
| coil | KKIAKMEKASSVFNWNSKKKC | 29 |
| tetrameric coiled | KLKQIEDKLEEILSKLYHIENELAKIEKKL | SEQ ID NO 30 |
| coil | AKMEKASSVFNWKKC | |

More preferably, the tandem heptad repeat motifs in the present invention consist of the sequence IEKKIE-X0 (SEQ TD NO: 115), wherein X0 represents an amino acid. In a preferred embodiment, said repeat motif consists of the sequence IEKKIE-X0, wherein X0 represents an amino acid provided that said X0 is not proline. In another preferred embodiment, said repeat motif consists of the sequence IEKKIE-X0, wherein X0 represents an amino acid, wherein said amino acid is a naturally occurring amino acid, wherein said naturally occurring amino acid is in its L-configuration, in its D-configuration, or in a mixture of any ratio thereof, provided that said amino acid is not proline. In another preferred embodiment, said repeat motif consists of the sequence IEKKIE-X0, wherein X0 represents an amino acid, wherein said amino acid is a naturally occurring amino acid in its L-configuration.

In a very preferred embodiment, said coiled coil peptide chain segment of said peptide moiety comprises or preferably consists of 2 to 10 repeat units, wherein said repeat units comprise or preferably consist of the IEKKIE-X0 (SEQ ID NO: 115). In a very preferred embodiment, said coiled coil peptide chain segment of said peptide moiety comprises or preferably consists of 2 to 10 repeat units, wherein said repeat units comprise or consist independently of each other of a sequence selected from IEKKIEG (SEQ ID NO: 116), IEKKIEA (SEQ ID NO: 117) or IEKKIES (SEQ ID NO: 118). In a very preferred embodiment, said coiled coil peptide chain segment of said peptide moiety comprises or preferably consists of 2 to 10 repeat units, wherein said repeat units comprise or consist independently of each other of a sequence selected from IEKKIEA (SEQ ID NO: 117) or IEKKIES (SEQ ID NO: 118).

In a preferred embodiment, said repeat motif consists of the sequence selected from IEKKIEG (SEQ ID NO: 116), IEKKIEA (SEQ ID NO: 117) or IEKKIES (SEQ ID NO: 118). In a preferred embodiment, said repeat motif consists of the sequence selected from SEQ ID NO: 117 or 118. In a very preferred embodiment, said repeat motif consists of the sequence IEKKIEA (SEQ ID NO: 117). Most preferably, said repeat motif consists of the sequence IEKKIES (SEQ ID NO: 118).

In a preferred embodiment, said coiled coil domain comprises of the sequence selected from (IEKKIE-X0)₄. In a preferred embodiment, said coiled coil domain comprises of the sequence selected from (IEKKIEG)₄ (SEQ ID NO: 119), (IEKKIEA)₄ (SEQ ID NO: 120) or (IEKKIES)₄ (SEQ ID NO: 121). In a preferred embodiment, said coiled coil domain consists of the sequence selected from SEQ ID NO: 119-121. In a very preferred embodiment, said coiled coil peptide chain segment comprises of the sequence SEQ ID NO: 120 or 121. In a very preferred embodiment, said coiled coil peptide chain segment of the peptide moiety consists of the sequence SEQ ID NO: 120 or 121. In a very preferred embodiment, said coiled coil peptide chain segment comprises, or preferably consists of, the sequence (IEKKIEA)₄ (SEQ ID NO: 120). In a most preferred embodiment, said coiled coil peptide chain segment of the peptide moiety consists of the sequence (IEKKIES)₄ (SEQ ID NO: 121).

Also preferred are coiled coil peptide sequences identified in naturally occurring peptides and proteins, but excluding those of human origin. These are, for example, coiled coils identified in viral and bacterial proteins. Also preferred are coiled coil peptide sequences disclosed in WO 2008/068017A1, WO 2015/082501A1, and WO2018/229156A1, WO 2020/127728 A1, the disclosure of these applications is incorporated herein in their entirety by way of reference,

T Helper Cell Epitope

The peptide chain may further comprise an amino acid sequence motif which includes one or more T-helper cell epitopes, and/or strings of polar residues that promote the solubility of the lipopeptide building blocks (LBB) and conjugates in water.

Thus, in a specific embodiment, said coiled-coil sequences may contain at least one T-helper cell epitope, preferably at least one universal T helper cell epitope. Preferably, said T helper cell epitope is present on the C terminus or the N terminus of the said coiled coil sequence. Preferably, said T helper cell epitope is present on the C terminus.

Preferably, said universal T helper cell epitope has the following sequence (SEQ ID NO: 31): R¹-Glu-Lys-Lys-Ile-Ala-Lys-Met-Glu-Lys-Ala-Ser-Ser-Val-Phe-Asn-Val-R² wherein:
R¹ is H-Asp-Ile-, H-Ile- or H—, and
R² is -Val-Asn-Ser-OH, -Val-Asn-OH, -Val-OH or —OH.

The T helper cell epitopes is selected from the group consisting of SEQ ID N^{o} 32, 33, 34, 35, 36, 37, 38, 39, 40, 41, 42 and 43. Said T helper cell epitopes are disclosed in the following table 2:

| H-Asp-Ile-Glu-Lys-Lys-Ile-Ala-Lys-Met-Glu-Lys-Ala-Ser-Ser-Val-Phe- | SEQ ID NO: 32 |
|---|---|
| Asn-Val-OH | |
| H-Asp-Ile-Glu-Lys-Lys-Ile-Ala-Lys-Met-Glu-Lys-Ala-Ser-Ser-Val-Phe- | SEQ ID NO: |
| Asn-Val-Val-OH | 33 |
| H-Asp-Ile-Glu-Lys-Lys-Ile-Ala-Lys-Met-Glu-Lys-Ala-Ser-Ser-Val-Phe- | SEQ ID NO: |
| Asn-Val-Val-Asn-OH | 34 |
| H-Ile-Glu-Lys-Lys-Ile-Ala-Lys-Met-Glu-Lys-Ala-Ser-Ser-Val-Phe-Asn- | SEQ ID NO: |
| Val-OH | 35 |
| H-Ile-Glu-Lys-Lys-Ile-Ala-Lys-Met-Glu-Lys-Ala-Ser-Ser-Val-Phe-Asn- | SEQ ID NO: |
| Val-Val-OH | 36 |
| H-Ile-Glu-Lys-Lys-Ile-Ala-Lys-Met-Glu-Lys-Ala-Ser-Ser-Val-Phe-Asn- | SEQ ID NO: |
| Val-Val-Asn-OH | 37 |
| H-Ile-Glu-Lys-Lys-Ile-Ala-Lys-Met-Glu-Lys-Ala-Ser-Ser-Val-Phe-Asn- | SEQ ID NO: |
| Val-Val-Asn-Ser-OH | 38 |
| H-Glu-Lys-Lys-Ile-Ala-Lys-Met-Glu-Lys-Ala-Ser-Ser-Val-Phe-Asn-Val | SEQ ID NO: |
| -OH | 39 |
| H-Glu-Lys-Lys-Ile-Ala-Lys-Met-Glu-Lys-Ala-Ser-Ser-Val-Phe-Asn-Val- | SEQ ID NO: |
| Val-OH | 40 |
| H-Glu-Lys-Lys-Ile-Ala-Lys-Met-Glu-Lys-Ala-Ser-Ser-Val-Phe-Asn-Val- | SEQ ID NO: |
| Val-Asn-OH | 41 |
| H-Glu-Lys-Lys-Ile-Ala-Lys-Met-Glu-Lys-Ala-Ser-Ser-Val-Phe-Asn-Val- | SEQ ID NO: |
| Val-Asn-Ser-OH | 42 |
| H-Asp-Ile-Glu-Lys-Lys-Ile-Ala-Lys-Met-Glu-Lys-Ala-Ser-Ser-Val-Phe- | SEQ ID NO: |
| Asn-Val-Val-Asn-Ser-OH | 43 |

In one embodiment, said T helper cell epitope is a comprised in the sequence of the coiled coil domain of the peptide chain. In another embodiment, T-cell epitopes may be incorporated into, or appended to the coiled-coil sequence of the peptide chain.

In a preferred embodiment, said peptide moiety further comprises a T-helper cell epitope, wherein said T-helper cell epitope comprises or preferably consists of a sequence selected from the group consisting of (i) SEQ TD NO: 89-114 and (ii) SEQ TD NO: 89-114, wherein one, two, or three amino acids are exchanged by other amino acids or are deleted. In a preferred embodiment, said peptide moiety further comprises a T-helper cell epitope, wherein said T-helper cell epitope consists of a sequence selected from the group consisting of (i) SEQ ID NO: 89-114 and (ii) SEQ TD NO: 89-114, wherein one, two, or three amino acids are exchanged by other amino acids or are deleted. In a preferred embodiment, said peptide moiety further comprises a T-helper cell epitope, wherein said T-helper cell epitope comprises a sequence selected from the group consisting of SEQ TD NO: 89-114. In a preferred embodiment, said T-helper cell epitope consists of a sequence selected from the group consisting of SEQ TD NO: 89-114.

Suitable T-helper cell epitopes are known to the skilled person in the art and are described, e.g., in Weber et al., Advanced Drug Delivery Reviews, 2009, 61:11, 965-976; Caro-Aguilar et al., Infect. Immun., 2002, 70:7, 3479-3492; Mishra et al., Immunology, 1993, 79:3, 362-367; Kobayashi et al., Cancer Research, 2000, 60:18, 5228-523; Fraser et al., Vaccine, 2014, 32:24, 2896-2903; Grabowska et al., Int. J. Cancer, 2015, 136:1, 212-224 and WO1998/023635A1. More preferred T-helper cell epitopes included in the peptide moiety are those listed in WO 2015/082501 such TT830-843, TT1064-1079, TT1084-1099, TT947-968, TT1174-1189, DTD271-290, DTD321-340, DTD331-350, DTD351-370, DTD411-430, DTD431-450, TT632-651, CTMOMP36-60, TraT1, TraT2, TraT3, HbcAg50-69, HbSAg19-33, HA307-319, MA17-31, MVF258-277, MVF288-302, CS.T3, SM Th, PADRE1 and PADRE2 as well as variants thereof in which one, two, or three amino acids are inserted, replaced by other amino acids or deleted.

Preferred T-helper epitopes that can be incorporated into said peptide moiety are any one selected from the group listed in the following table below, and variants thereof in which one, two, or three amino acids are replaced by other amino acids or are deleted.

| T-helper | SEQ | Sequence^{a)} |
|---|---|---|
| epitope | ID NO: | |
| CS.T3 | 89 | IEKKIAKMEKASSVFNVVNS |
| TT830-843 | 90 | QYIKANSKFIGITE |
| TT1064-1079 | 91 | IREDNNITLKLDRCNN |
| TT1084-1099 | 92 | VSIDKFRIFCKANPK |
| TT947-968 | 93 | FNNFTVSFWLRVPKVSASHLET |
| TT1174-1189 | 94 | LKFIIKRYTPNNEIDS |
| DTD271-290 | 95 | PVFAGANYAAWAVNVAQVID |
| DTD321-340 | 96 | VHHNTEEIVAQSIALSSLMV |
| DTD331-350 | 97 | QSIALSSLMVAQAIPLVGEL |
| DTD351-370 | 98 | VDIGFAAYNFVESIINLFQV |
| DTD411-430 | 99 | QGESGHDIKITAENTPLPIA |
| DTD431-450 | 100 | GVLLPTIPGKLDVNKSKTHI |
| TT632-651 | 101 | TIDKISDVSTIVPYIGPALN |
| CTMOMP36-60 | 102 | ALNIWDRFDVFCTLGATTGYLKGNS |
| TraT1 | 103 | GLOGKIADAVKAKG |
| TraT2 | 104 | GLAAGLVGMAADAMVEDVN |
| TraT3 | 105 | STETGNQHHYQTRVVSNANK |
| HbcAg50-69 | 106 | PHHTALRQAILCWGELMTLA |
| HbSAg19-33 | 107 | FFLLTRILTIPQSLD |
| HA307-319 | 108 | PKYVKQNTLKLAT |
| MA17-31 | 109 | YSGPLKAEIAQRLEDV |
| MVF258-277 | 110 | GILESRGIKARITHVDTESY |
| MVF288-302 | 111 | LSEIKGVIVHRLEGV |
| SM Th | 112 | KWFKTNAPNGVDEKIRI |
| PADRE1^{b)} | 113 | aKFVAAWTLKAAa |
| PADRE2^{b)} | 114 | aK-Chx-VAAWTLKAAa |

^{a)}References: SEQ ID NO: 63-67 and 17-20: Eur. J. Immunol. 2001, 31, 3816-3824; SEQ ID NO: 68-74: JID 2000, 181, 1001-1009; SEQ ID NO: 75-78, 83-84 and 85: US 5,759,551; SEQ ID NO: 6: Nature 1988, 336, 778-780; SEQ ID NO: 86-87: Immunity 1994, 1, 751-761.
^{b)}_{”}a^{“} denotes D-Ala and _{”}Chx^{“} denotes cyclohexylalanine.

In a most preferred embodiment, the T-helper cell epitope comprises or preferably consists of the following amino acid sequence: IEKKIAKMEKASSVFNVVNS (SEQ ID NO: 89).

In a further very preferred embodiment, said peptide moiety comprises or preferably consists of GG(IEKKIES)₄IEKKIAKMEKASSVFNVVNSKKKC (SEQ IDNO: 127) or GG(IEKKIEA)₄IEKKIAKMEKASSVFNVVNSKKKC (SEQ ID NO: 128), more preferably SEQ ID NO: 127. In a further very preferred embodiment, said peptide moiety consists of SEQ ID NO: 127 or 128, more preferably SEQ ID NO: 127.

In a further preferred embodiment, said peptide moiety comprises (i) an N-terminal amino acid sequence, wherein said N-terminal amino acid sequence comprises or preferably consists of fibroblast-stimulating lipopeptide FSL-1 (S-(2,3-bispalmitoyloxypropyl)- or PAM2-Cys-Gly-Asp-Pro-Lys-His-Pro-Lys-Ser-Phe; SEQ ID NO: 122), FSL-2 (S-(2,3-bispalmitoyloxypropyl)- or PAM2-Cys-Gly-Asp-Pro-Lys-His-Pro-Lys-Ser-Arg; SEQ ID NO: 123), FSL-3 (S-(2,3-bisstearyloxypropyl)-Cys-Gly-Asp-Pro-Lys-His-Pro-Lys-Ser-Phe; SEQ ID NO: 124), Mycoplasma fermentans-derived peptide MALP-2 (S-(2,3-bispalmitoyloxypropyl)- or PAM2-Cys-Gly-Asn-Asn-Asp-Glu-Ser-Asn-Ile-Ser-Phe-Lys-Glu-Lys; SEQ ID NO: 125), or GG; and/or GX where X is Asx or Ser and/or (ii) a C-terminal amino acid sequence, wherein said C-terminal amino acid sequence comprises or preferably consists of a sequence recognized by an enzyme as cleavage site; wherein preferably said C-terminal amino acid sequence comprises or preferably consists of sequence KKKCa (SEQ ID NO: 126) or wherein preferably said C-terminal amino acid sequence is an amino acid sequence of consecutive 5 amino acids.

In another preferred embodiment, said peptide moiety comprises or preferably consists of SEQ ID NO: 129.

Further preferred are T helper epitopes are disclosed in WO 2008/068017A1, WO 2015/082501A1, and WO2018/229156A1, WO 2020/127728 A1, the disclosure of these applications is incorporated herein in their entirety by way of reference,

Lipid Moiety (LM)

The covalently linked peptide moiety and lipid moiety form a lipopeptide referred to herein also as lipopeptide building block (LBB). The presence of the lipid moiety facilitates presentation of the epitope to B cells, since it is known that antigens associated with membranes are particularly effective at activating B-cells and promoting B cell-driven T cell activation. The high local concentration of lipid moieties present within the assembled HLB and SVLP will facilitate interaction of the assembly with membranes and promote presentation of antigens to B cells. The lipid portion of the LBB may be derived from bacterially derived lipid moieties, such as the well-known lipopeptide Toll-like receptor ligands.

Typically, said lipid moiety contains a lipid anchor with two or three, preferably two, long hydrocarbyl chains and a structure combining these hydrocarbyl chains and connect it to the peptide chain (PC), either directly or via a connecting moiety. Preferred lipid moieties are phospholipids containing two or three, preferably two extended hydrocarbyl chains.

“Long hydrocarbyl chain”, “hydrocarbyl chain”, “Long hydrocarbyl” or “hydrocarbyl” means a straight alkyl or alkenyl group of at least 7 carbon atoms, for example straight alkyl or alkenyl consisting of between 8 and 50 C atoms, preferably between 8 and 25 C atoms. Alkenyl has preferably one, two or three double bonds in the chain, each with E or Z geometry, as is customarily found in natural fatty acids and fatty alcohols. Also included in the definition of “long hydrocarbyl” is branched alkyl or alkenyl, for example alkyl bearing a methyl or ethyl substituent at the second or third carbon atom counted from the end of the chain, as e.g. in 2-ethyl-hexyl.

Preferred lipid moieties according to the invention are those of formula Z¹ to Z⁸.

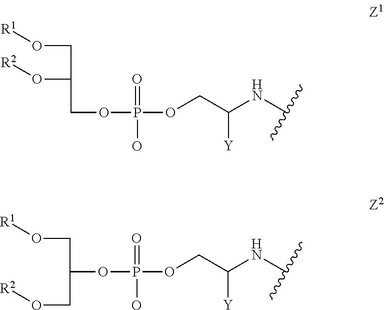

wherein R¹ and R² are long hydrocarbyl or long hydrocarbyl-C═O and Y is H or COOH,

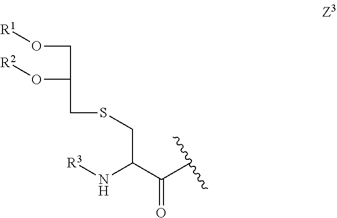

wherein R¹, R² and R³ are long hydrocarbyl or R¹ and R² are long hydrocarbyl-C═O and R³ is H or acetyl,

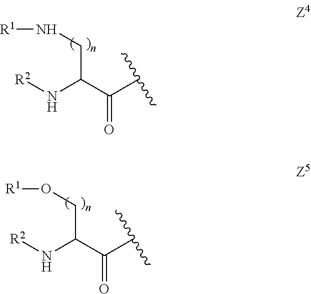

wherein R¹ and R² are long hydrocarbyl-C═O and n is 1, 2, 3 or 4,

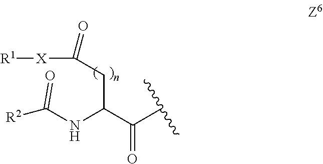

wherein R¹ and R² are long hydrocarbyl, X is O or NH, and n is 1, 2, 3 or 4, or

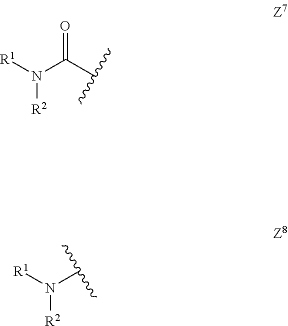

wherein R¹ and R² are long hydrocarbyl.

Preferably, said lipid moiety is one of formulas Z1 to Z8, wherein R¹ and R² in formulas Z1 and Z2 are independently of each other hydrocarbyl or hydrocarbyl-C═O, and Y is H or COOH; wherein R¹, R² and R³ in formula Z3 are independently of each other hydrocarbyl or hydrocarbyl-C═O; or R¹ and R² are independently of each other hydrocarbyl or hydrocarbyl-C═O, and R³ is H or acetyl or lower alkyl-C═O; wherein R¹ and R² in formulas Z4 and Z5 are independently of each other hydrocarbyl or hydrocarbyl-C═O, and n is 1, 2, 3 or 4; wherein R¹ and R² in formula Z6 are independently of each other a hydrocarbyl, X is O or NH, and n is 1, 2, 3 or 4, or wherein R¹ and R² in formulas Z7 and Z8 are independently of each other hydrocarbyl. A preferred lipid moiety is di-palmitoyl-S-glycerylcysteinyl of formula LM3, wherein R¹ and R² are palmitoyl, and R³ is H or acetyl. Preferably, the term, “lower alkyl” means alkyl with 1 to 7 carbon atoms, more preferably 1 to 4 carbon atoms, such as methyl, ethyl, n-propyl, iso-propyl, n-butyl, sec-butyl, iso-butyl or tert-butyl.

The lipid moiety contains at least two long hydrocarbyl chains such as found in fatty acids, e.g. as in Z¹ to Z⁸. One preferred lipid moiety is a phospholipid of various types, e.g. of formula Z¹ or Z², that possess either ester or ether-linked extended alkyl or alkenyl chains, such as either enantiomer of 1,2-dipalmitoyl-sn-glycero-3-phosphoethanolamine, or achiral analogues such as 1,3-dipalmitoyl-glycero-2-phosphoethanolamine. A preferred lipid moiety is a tri- or di-palmitoyl-S-glycerylcysteinyl residue (type Z³) or lipid moieties of types Z⁴ to Z⁸.

In a preferred embodiment, said lipid moiety of the conjugate of the invention (herein mentioned also as LM) preferably consisting of, the formula LM-I

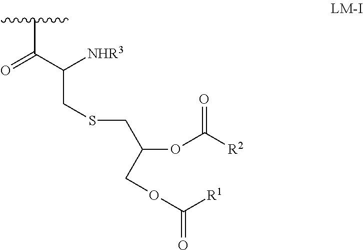

wherein R¹ and R² are independently C₁₁₋₁₅alkyl, preferably R¹ and R² are independently —C₁₁H₂₃, —C₁₃H₂₇ or —C₁₅H₃₁, and further preferably R¹ and R² are —C₁₅H₃₁; and R³ is hydrogen or —C(O)C₁₁₋₁₅alkyl, preferably R³ is H or —C(O)C₁₅H₃₁.

In another preferred embodiment, said lipid moiety of the conjugate of the invention (herein mentioned also as LM) preferably consisting of, the formula LM-II

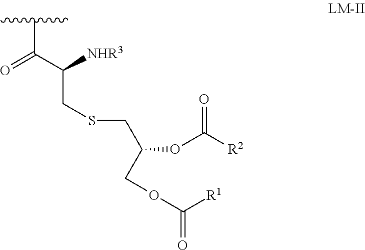

wherein R¹ and R² are independently C₁₁₋₁₅alkyl, preferably R¹ and R² are independently —C₁₁H₂₃, —C₁₃H₂₇ or —C₁₅H₃₁, and further preferably R¹ and R² are —C₁₅H₃₁; and R³ is hydrogen or —C(O)C₁₁₋₁₅alkyl, preferably R³ is H or —C(O)C₁₅H₃₁.

Lipopeptide building blocks comprising Pam₂Cys or Pam₃Cys moieties with the (R)-configuration at the 2-propyl carbon atom and further comprising as coiled coil peptide chain segment several units of the sequence IEKKIE-X0 with preferably X0 being Gly, Ala or Ser, most preferably Ser, provide increased avidity of the antibodies generated against said RSV-F protein, said variant, or said fragment thereof linked to the lipopeptide building blocks and comprised by the inventive conjugates or SVLPs, respectively.

Said lipid moiety is linked to said peptide moiety, wherein the wavy line in formula LM-I and LM-II and other LM formulas mentioned herein indicates the linkage site to said peptide moiety.

In a preferred embodiment, said R¹ and R² are independently —C₁₁H₂₃, —C₁₃H₂₇ or —C₁₅H₃₁. In a very preferred embodiment, said R¹ and R² are —C₁₅H₃₁. In a preferred embodiment, said R³ is H or —C(O)C₁₅H₃₁. In a preferred embodiment, said R¹ and R² are independently —C₁₁H₂₃, —C₁₃H₂₇ or —C₁₅H₃₁, and R³ is hydrogen or —C(O)C₁₁₋₁₅alkyl. In a very preferred embodiment, said R¹ and R² are —C₁₅H₃₁, and R³ is hydrogen or —C(O)C₁₁₋₁₅alkyl. In a preferred embodiment, said R¹ and R² are independently —C₁₁H₂₃, —C₁₃H₂₇ or —C₁₅H₃₁, and R³ is H or —C(O)C₁₅H₃₁. In a very preferred embodiment, said R¹ and R² are —C₁₅H₃₁, and R³ is H or —C(O)C₁₅H₃₁.

In a preferred embodiment, said lipid moiety is linked to the N-terminus of said peptide moiety. This conveniently allows that said linking can be performed on-resin after assembly of the peptide chain of said peptide moiety by solid phase peptide synthesis. Linking of said lipid moiety to the C-terminus of said peptide moiety is also encompassed within the present invention and is possible using linkage chemistry known by the skilled person in the art.

A preferred lipid moiety is di-palmitoyl-S-glycerylcysteinyl (Pam₂Cys) or tripalmitoyl-S-glyceryl cysteine (Pam₃Cys), more preferably, Pam₂Cys. More preferably, Pam₂Cys or Pam₃Cys are both with the R-configuration at the chiral 2-propyl carbon atom and the R-configuration of the chiral carbon of the cysteinyl moiety.

In a preferred embodiment, said lipid moiety comprises, preferably consists of, the formula LM-I*

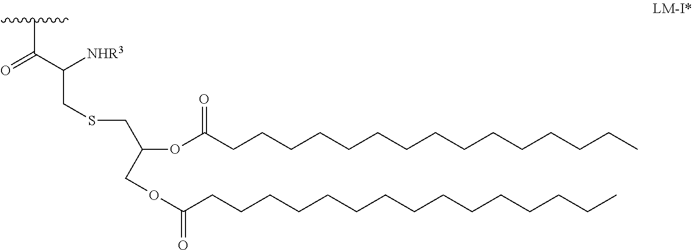

wherein R³ is hydrogen or —C(O)C₁₁₋₁₅alkyl, preferably H or —C(O)C₁₅H₃₁; wherein preferably said lipid moiety is linked to the N-terminus of said peptide moiety.

In a preferred embodiment, said lipid moiety comprises, preferably consists of, the formula LM-II*

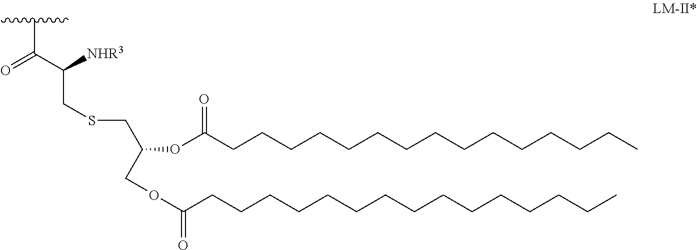

wherein R³ is hydrogen or —C(O)C₁₁₋₁₅alkyl, preferably H or —C(O)C₁₅H₃₁; wherein preferably said lipid moiety is linked to the N-terminus of said peptide moiety.

In a preferred embodiment, said lipid moiety consists of the formula LM-I* or LM-II*, wherein R³ is hydrogen or —C(O)C₁₁₋₁₅alkyl. In a preferred embodiment, said lipid moiety comprises, preferably consists of, the formula LM-I* or LM-II*, wherein R³ is H or —C(O)C₁₅H₃₁. In a preferred embodiment, said lipid moiety comprises, preferably consists of, the formula LM-I* or LM-II*, wherein R³ is H or —C(O)C₁₅H₃₁ and wherein said lipid moiety is linked to the N-terminus of said peptide moiety. In a preferred embodiment, said lipid moiety consists of, the formula LM-I* or LM-II*, wherein R³ is H or —C(O)C₁₅H₃₁ and wherein preferably said lipid moiety is linked to the N-terminus of said peptide moiety.

In a very preferred embodiment, said lipid moiety comprises, preferably consists of, the formula LM-I*1 or LM-I*2. In a very preferred embodiment, said lipid moiety consists of the formula LM-I*1 or LM-I*2.

In a very preferred embodiment, said lipid moiety consists of the formula LM-I*1.

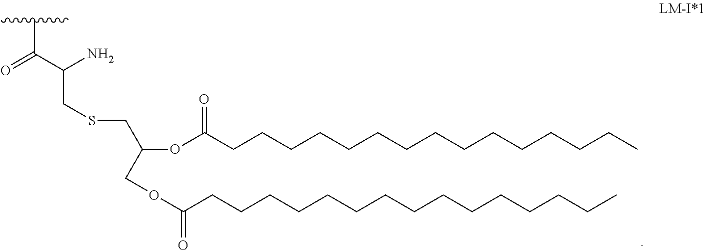 [SMILES: *C(=O)C(N)CSCC(COC(=O)CCCCCCCCCCCCCCC)OC(=O)CCCCCCCCCCCCCCC]

In a very preferred embodiment, said lipid moiety consists of the formula LM-II*1.

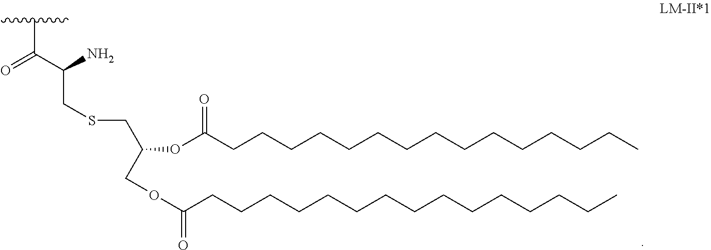 [SMILES: *C(=O)[C@@H](N)CSC[C@@H](COC(=O)CCCCCCCCCCCCCCC)OC(=O)CCCCCCCCCCCCCCC]

In a very preferred embodiment, said lipid moiety consists of the formula LM-I*2.

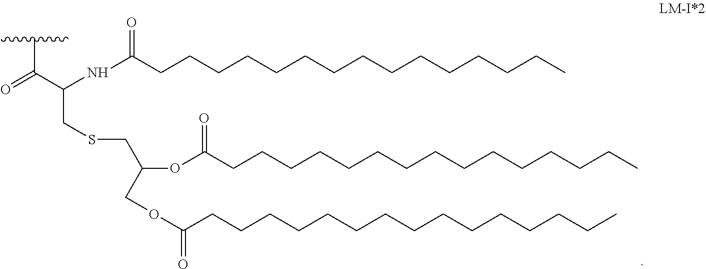 [SMILES: *C(=O)C(CSCC(COC(=O)CCCCCCCCCCCCCCC)OC(=O)CCCCCCCCCCCCCCC)NC(=O)CCCCCCCCCCCCCCC]

Very preferred lipid moieties of the present invention are thus Pam₃Cys LM-II*2, i.e. tripalmitoyl-S-glyceryl cysteine (N-palmitoyl-S-[(2,3-bis-(O-palmitoyloxy)-(2-propyl)]-cysteinyl-) or Pam₂Cys LM-II*1, i.e. dipalmitoyl-S-glyceryl cysteine (S-[2,3-bis-(O-palmitoyloxy)-(2-propyl)]-cysteinyl-). In a most preferred embodiment, said lipid moiety is N-α-palmitoyl-S-[2,3-bis(palmitoyloxy)-(2-propyl)]-cysteine or S-[2,3-bis(palmitoyloxy)-(2-propyl)]-cysteine, thus LM-II*1.

In a very preferred embodiment, said lipid moiety consists of the formula LM-II*2.

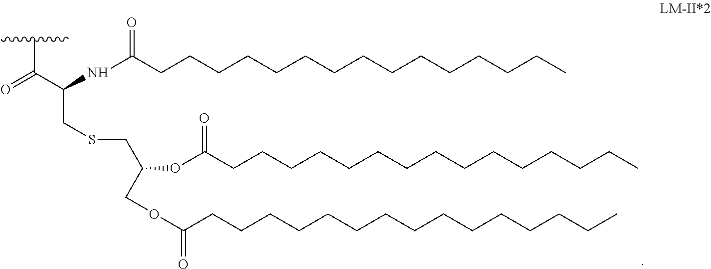 [SMILES: *C(=O)[C@H](CSC[C@@H](COC(=O)CCCCCCCCCCCCCCC)OC(=O)CCCCCCCCCCCCCCC)NC(=O)CCCCCCCCCCCCCCC]

Very preferred lipid moieties of the present invention are, thus, (R,R)-Pam₃Cys LM-II*2, i.e. tripalmitoyl-S-glyceryl cysteine (N-palmitoyl-S-[2,3-bis-(O-palmitoyloxy)-(2R)-propyl]-(R)-cysteinyl-) and (R,R)-Pam₂Cys LM-II*1, i.e. dipalmitoyl-S-glyceryl cysteine (S-[2,3-bis-(O-palmitoyloxy)-(2R)-propyl]-(R)-cysteinyl-). Thus, in a further very preferred embodiment, said lipid moiety is N-α-Palmitoyl-S-[2,3-bis(palmitoyloxy)-(2R)-propyl]-(R)-cysteine or S-[2,3-bis(palmitoyloxy)-(2R)-propyl]-(R)-cysteine, thus LM-II*1.

In a preferred embodiment, the lipid moiety is linked to the peptide moiety, either directly or via a connecting moiety. Preferably, the lipid moiety is linked to the peptide moiety at or near one terminus, i.e. the N-terminus or the C-terminus, preferably the N-terminus. In a preferred embodiment, the lipid moiety is linked to the first, second, third, fourth or fifth amino acid of the peptide moiety, calculated from the N-terminus or C-terminus of the peptide moiety. The lipid moiety may be linked, directly or through a connecting moiety, to the backbone or to the side chain of one of the amino acids of the peptide moiety, preferably said amino acid is near to the terminus, more preferably it is the first, second, third, fourth or fifth amino acid of the peptide moiety.

If the peptide moiety and the lipid moiety are directly linked, this is preferably accomplished through an amide bond between a lipid moiety carbonyl function and an amino function, e.g. the N-terminal amino function, of the peptide moiety. It will be apparent to the skilled person in the art that a large variety of suitable connecting moieties and strategies exist, which include but are not limited to connecting moieties based on dicarboxylic acid derivatives, connecting moieties containing one or multiple ethylene glycol units, amino acid residues (including alpha-, beta-, gamma-, omega-amino acids), or sugar (carbohydrate) units, or containing heterocyclic rings.

In a preferred embodiment, said lipid moiety and said peptide moiety are linked via a connecting moiety. Preferably, said linking of said lipid moiety and said peptide moiety via said connecting moiety is by way of an amide bond between a carbonyl function of said lipid moiety and an amino function of said connecting moiety. Preferably, said amino function is the N-terminal amino function of said peptide moiety.

In a preferred embodiment, said lipid moiety and said peptide moiety are linked via a connecting moiety, by way of an amide bond between a carbonyl function of said lipid moiety and an amino function of said connecting moiety, wherein said connecting moiety is an amino acid linker consisting of 2-15 more preferably 2-10, again more preferably 2-5 amino acids. Examples hereto include the amino acid linker sequences comprised by FSL-1, FSL-2, FSL-3, PAM2 or MALP-2 moieties. In a preferred embodiment, said lipid moiety and said peptide moiety are linked via a connecting moiety, wherein said connecting moiety is a Gly-Gly moiety.

In a preferred embodiment, two Gly residues are included as connecting moiety between the lipid moiety, preferably said (R,R)-Pam2Cys moiety LM-I*1 or LM-II*1 and the start of the coiled-coil heptad repeats, typically and preferably the coiled coil peptide chain segment comprising, preferably consisting of, the sequence IEKKIES or IEKKIEA. Further preferred lipid moieties are disclosed in WO 2018/229156 A1 and WO 2020/127728 A1, the disclosure of these applications is incorporated herein in their entirety by way of reference,

The peptide chain (PC) is covalently linked to the lipid moiety (LM) at or near one terminus, i.e. the N terminus or the C terminus, preferably the N terminus. The lipid moiety may be directly attached (below formula (1)) or via a connecting moiety (below formulas (2) or (3)), wherein L means connecting moiety and X is O or NH.

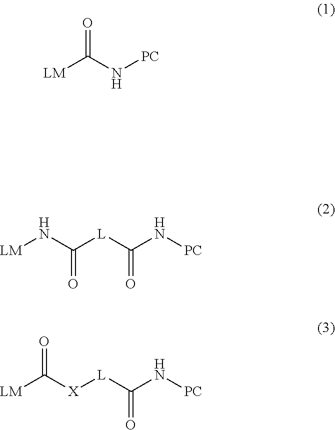

Suitable connecting moieties include but are not limited to connecting moieties based on dicarboxylic acid derivatives, connecting moieties containing one or multiple ethylene glycol units, amino acid residues (including α-, β-, γ-, δ-amino acids etc.), or sugar (carbohydrate) units, or containing heterocyclic rings. Particular connecting moieties considered are connecting moieties L¹ to L¹⁰, wherein n is between 1 and 20 and m is between 1 and 20, shown with the connecting functional group C═O or X wherein X is O or NH:

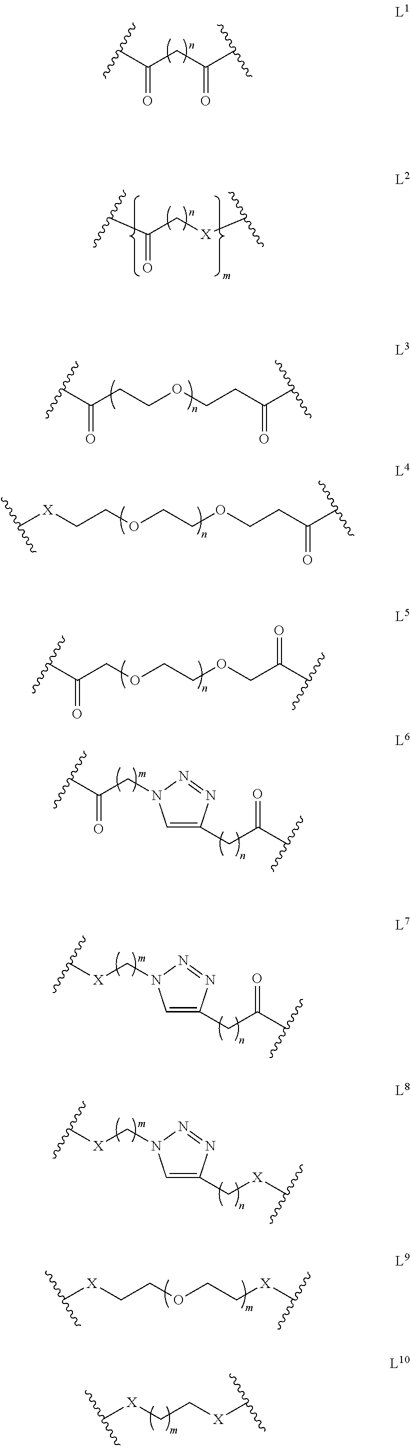

“Near one terminus” as understood in this connection means that the lipid moiety is bound to the first, second, third, fourth or fifth amino acid calculated from the N terminal or C terminal end, respectively, of the peptide. The lipid moiety may be attached to the backbone of the peptide structure or to the side chain of one of these amino acids near to the terminus.

The antigen of the invention is an RSV-F protein, a variant or a fragment thereof and is used to elicit an antigen-specific humoral immune response. The antigen of the invention is covalently attached at or near the other end of the peptide chain, whereby “other” means the end of the peptide not carrying the lipid moiety. If the lipid moiety is connected at or near the N terminus of the peptide, then the antigen is bound at or near the C terminus. If the lipid moiety is connected at or near the C terminus of the peptide, then the antigen is bound at or near the N terminus.

One or more antigens of the invention may be conjugated to the coiled-coil domain of the peptide chain (PC), for example, through one or more of the side chains of amino acids in the coiled-coil peptide (e.g. lysine, or cysteine), or through the chain terminus of the peptide chain. Said antigen carries a functional group suitable for conjugation to a functional group in one of the side chains or the terminus of the peptide chain.

Lipopeptide Building Block (LBB)

In a preferred embodiment, a lipopeptide building block consists of
(i) a peptide moiety comprising a coiled coil peptide chain segment, wherein said coiled coil peptide chain segment comprises 3 to 8 repeat units, and wherein said repeat unit consists of the sequence IEKKIE-X0 (SEQ ID NO: 115), wherein X0 represents an amino acid, and wherein preferably said repeat unit consists of the sequence selected from IEKKIEG (SEQ ID NO: 116), IEKKIEA (SEQ ID NO: 117) or IEKKIES (SEQ ID NO: 118), and wherein further preferably said repeat unit consists of the sequence IEKKIES (SEQ ID NO: 118);
(ii) a lipid moiety comprising, preferably consisting of, the formula LM-I

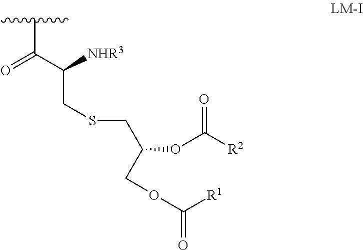

wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁; and wherein said lipid moiety is linked to said peptide moiety, wherein the wavy line in formula LM-I indicates the linkage site to said peptide moiety, and wherein preferably said lipid moiety is linked to the N-terminus of said peptide moiety.

In a preferred embodiment, said R¹ and R² are independently —C₁₁H₂₃, —C₁₃H₂₇ or —C₁₅H₃₁, and R³ is hydrogen or —C(O)C₁₁₋₁₅alkyl. In a very preferred embodiment, said R¹ and R² are —C₁₅H₃₁, and R³ is hydrogen or —C(O)C₁₁₋₁₅alkyl. In a preferred embodiment, said R¹ and R² are independently —C₁₁H₂₃, —C₁₃H₂₇ or —C₁₅H₃₁, and R³ is H or —C(O)C₁₅H₃₁. In a very preferred embodiment, said R¹ and R² are —C₁₅H₃₁, and R³ is H or —C(O)C₁₅H₃₁.

In a further very preferred embodiment, said lipopeptide building block is of the formula LBB-1 to LBB-6, preferably of LBB-1 to LBB-3, again more preferably LBB-2 and 3, most preferably LBB-2.

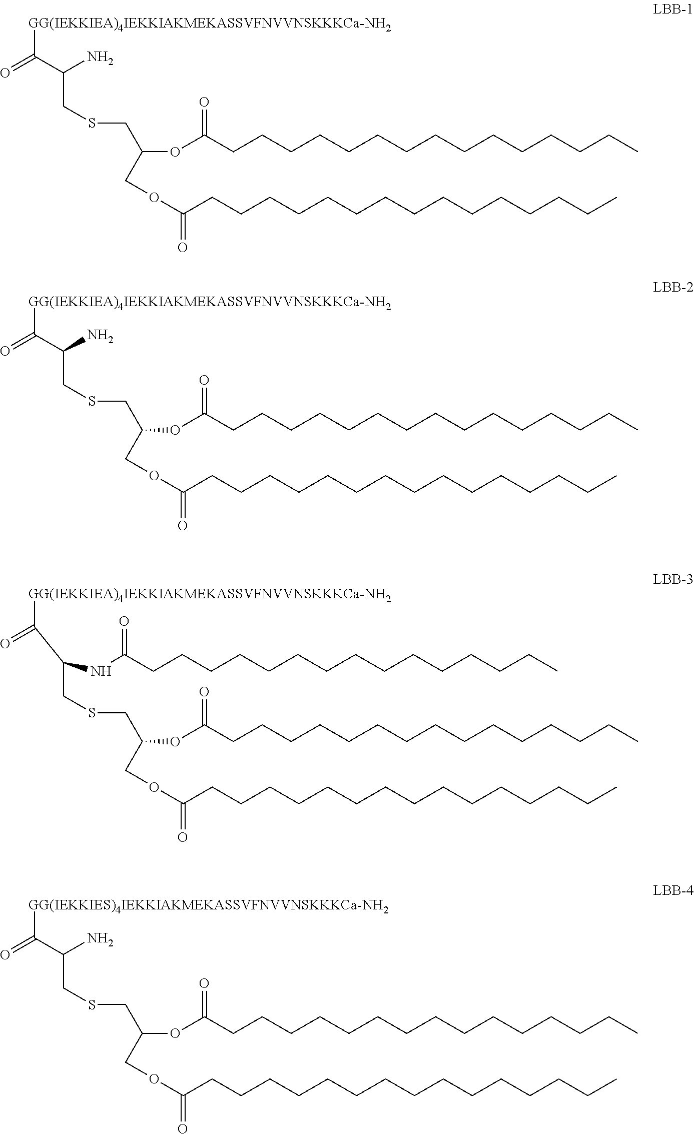

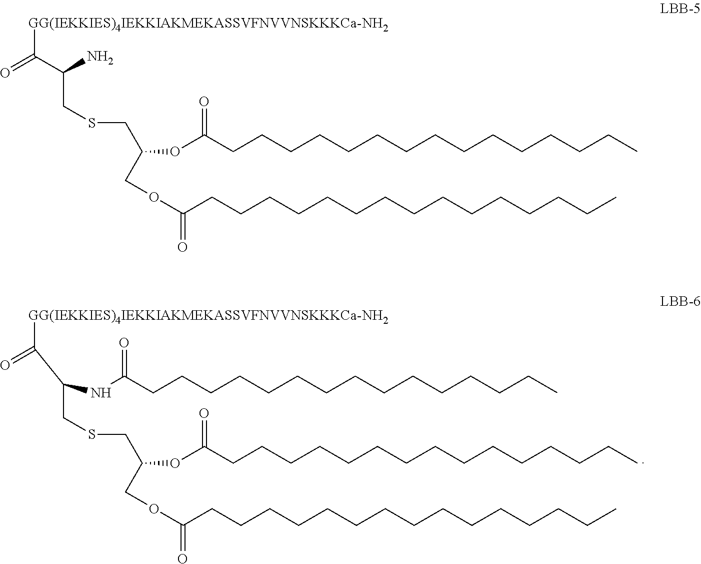

In a further very preferred embodiment, said lipopeptide building block is of the formula LBB-4. In a most preferred embodiment, said lipopeptide building block is of the formula LBB-5.

In further very preferred embodiment, the present invention provides a lipopeptide building block consisting of (i) a peptide moiety comprising a coiled coil peptide chain segment, wherein said coiled coil peptide chain segment comprises 3 to 8 repeat units, and wherein said repeat unit consists of the sequence IEKKIE-X0, wherein X0 represents an amino acid, and wherein preferably said repeat unit consists of the sequence selected from IEKKIEG, IEKKIEA or IEKKIES, and wherein further preferably said repeat unit consists of the sequence IEKKIES; (ii) a lipid moiety comprising, preferably consisting of, the formula LM-I or LM-II, wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁; and wherein said lipid moiety is linked to said peptide moiety, wherein the wavy line in formula LM-I indicates the linkage site to said peptide moiety, and wherein preferably said lipid moiety is linked to the N-terminus of said peptide moiety.

In further very preferred embodiment, the present invention provides a lipopeptide building block consisting of (i) a peptide moiety comprising a coiled coil peptide chain segment, and wherein said coiled coil peptide chain segment comprises, preferably consists of, the sequence of SEQ ID NO: 120 or 121; (ii) a lipid moiety comprising, preferably consisting of, the formula LM-I or LM-II, wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁; and wherein said lipid moiety is linked to said peptide moiety, wherein the wavy line in formula LM-I and LM-II indicates the linkage site to said peptide moiety, and wherein preferably said lipid moiety is linked to the N-terminus of said peptide moiety.

In an even more preferred embodiment, the present invention provides a lipopeptide building block consisting of (i) a peptide moiety comprising a coiled coil peptide chain segment, and wherein said coiled coil peptide chain segment comprises, preferably consists of, the sequence of SEQ ID NO: 120 or 121; (ii) a lipid moiety comprising, preferably consisting of, the formula LM-II, wherein R¹ and R² are independently C₁₁₋₁₅alkyl, wherein preferably R¹ and R² are independently —C₁₁H₂₃, —C₁₃H₂₇ or —C₁₅H₃₁, and wherein further preferably R¹ and R² are —C₁₅H₃₁; and wherein R³ is hydrogen or —C(O)C₁₁₋₁₅alkyl, and wherein preferably R³ is H or —C(O)C₁₅H₃₁; and wherein said lipid moiety is linked to said peptide moiety, wherein the wavy line in formula LM-II indicates the linkage site to said peptide moiety, and wherein preferably said lipid moiety is linked to the N-terminus of said peptide moiety.

Further preferred are LBB disclosed in WO2018/229156A1 and WO 2020/127728 A1, the disclosure of these applications is incorporated herein in their entirety by way of reference,

Conjugate

In a further aspect, the present invention provides a conjugate comprising (a) a lipopeptide building block and (b) an RSV-F protein, a variant or a fragment thereof, wherein said lipopeptide building block consists of (i) a peptide moiety comprising at least one coiled coil peptide chain segment, and (ii) a lipid moiety comprising two or three, preferably two hydrocarbyl chains; wherein said RSV-F protein, said variant or said fragment thereof is conjugated, directly or via a linker, to said lipopeptide building block. Preferably said RSV-F protein, variant or fragment is a variant of the RSV-F protein, wherein said variant is a cyclic peptide comprising said amino acid sequence (I) comprising or consisting of SEQ ID NO: 44. Preferably, said SEQ ID NO: 44 is a sequence selected from the group consisting of SEQ ID NO: 45-88, more preferably, SEQ ID NO: 45-83, again more preferably, SEQ ID NO: 45-64, again more preferably, SEQ ID NO: 45-51.

A variety of coupling or conjugation procedures may be used to attach the RSV-F protein, the variant or the fragment thereof to the peptide moiety, which will be well known to those knowledgeable in the field. Thus, free amino groups in the side chains of amino acids in the peptide moiety of the LBB may be coupled to reactive esters in the RSV-F protein, the variant or fragment thereof (e.g. N-hydroxysuccinimide esters prepared from carboxylic acids); thiols in the peptide moiety may be coupled to maleimide groups in the RSV-F protein, the variant or fragment thereof, azides may be incorporated into the side chains of amino acid residues in the peptide moiety and coupled to the RSV-F protein, the variant or fragment thereof containing acetylene groups using copper catalyzed cycloaddition reactions; and other nucleophiles (e.g. hydrazino, hydroxylamino, vic-aminothiol groups) in the peptide may be coupled to electrophiles (e.g. aldehydes, ketones, active esters) in the RSV-F protein, the variant or fragment thereof. It will be obvious that it is possible, in principle, to reverse the positions of the two reactive groups in the peptide chain and antigen in order to achieve selective coupling.

In a very preferred embodiment, said conjugate is selected from any one of the formula:

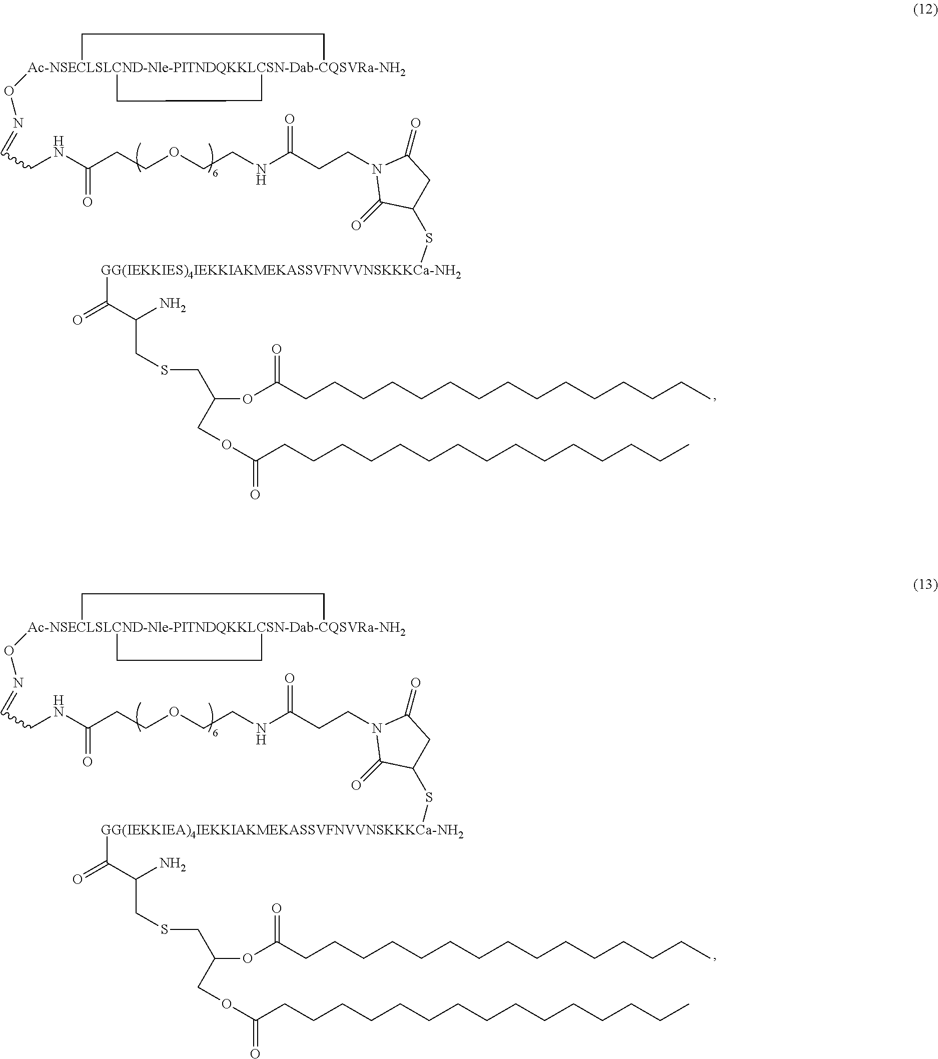

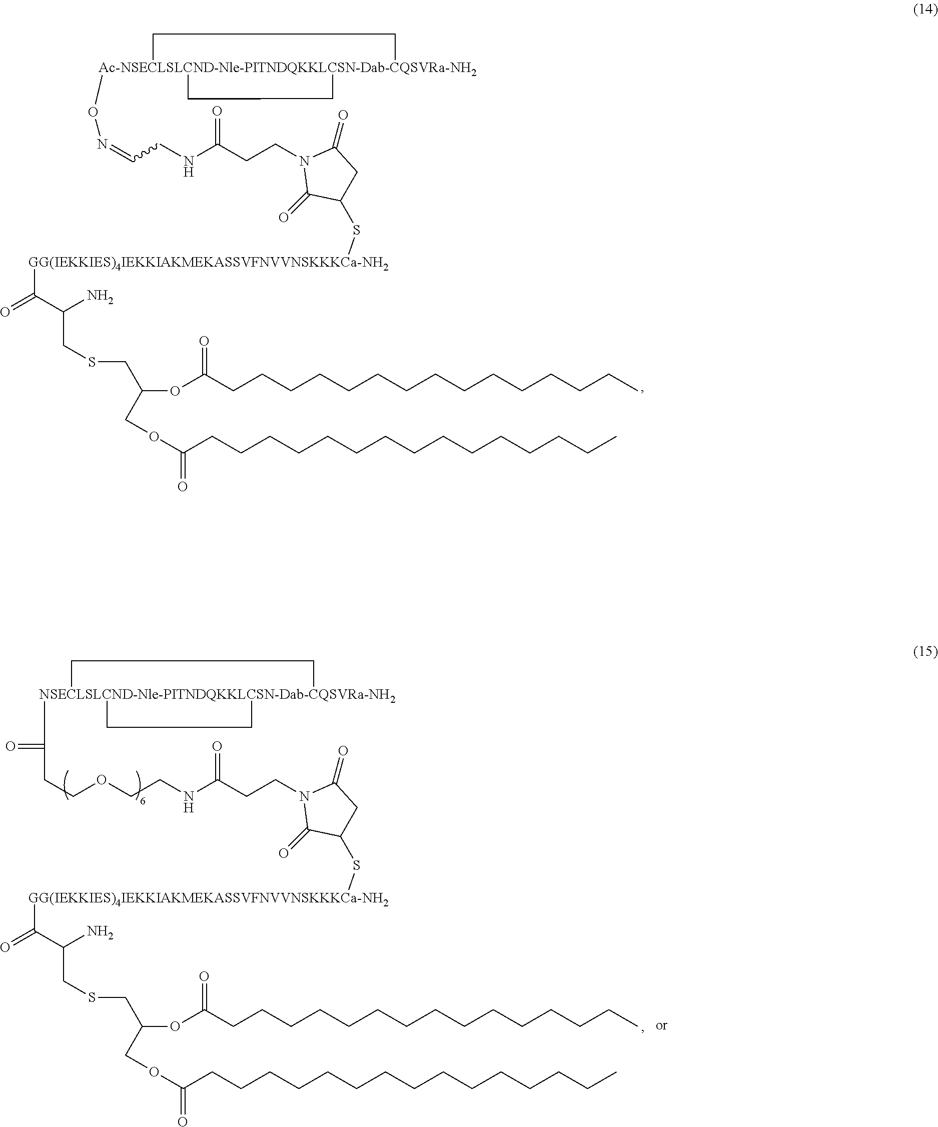

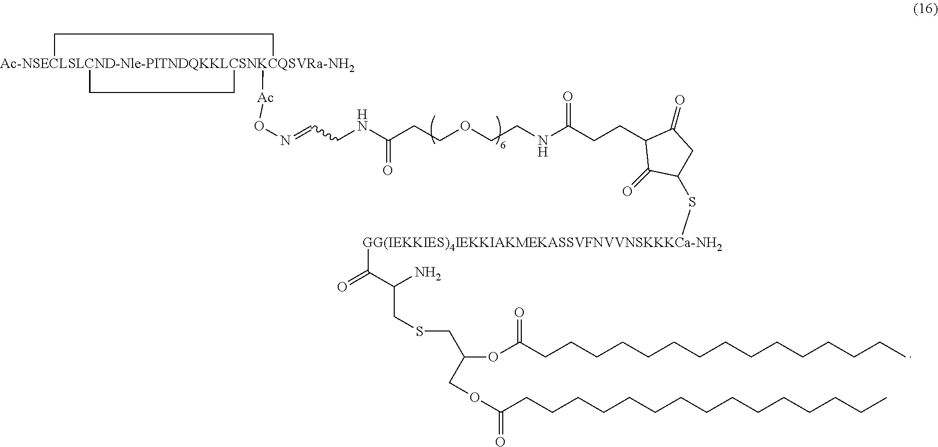

In a further very preferred embodiment, said conjugate is selected from any one of the formula

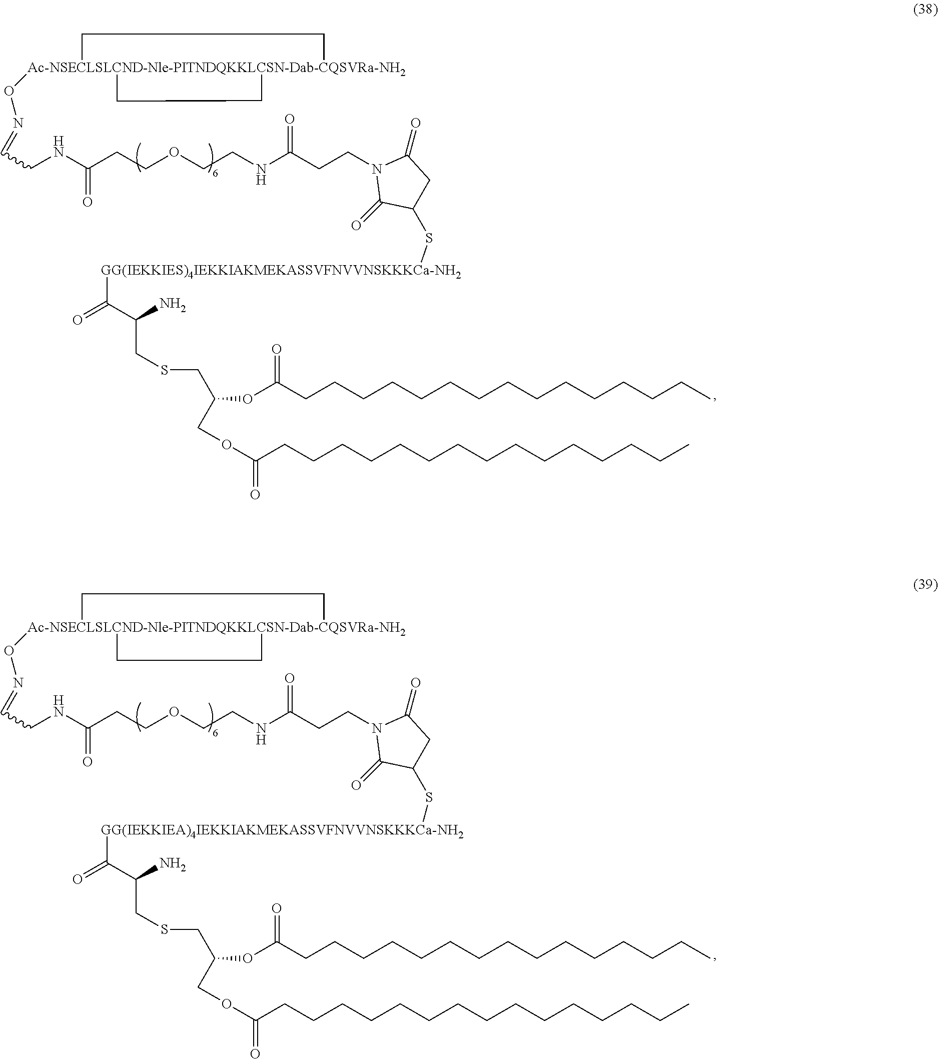

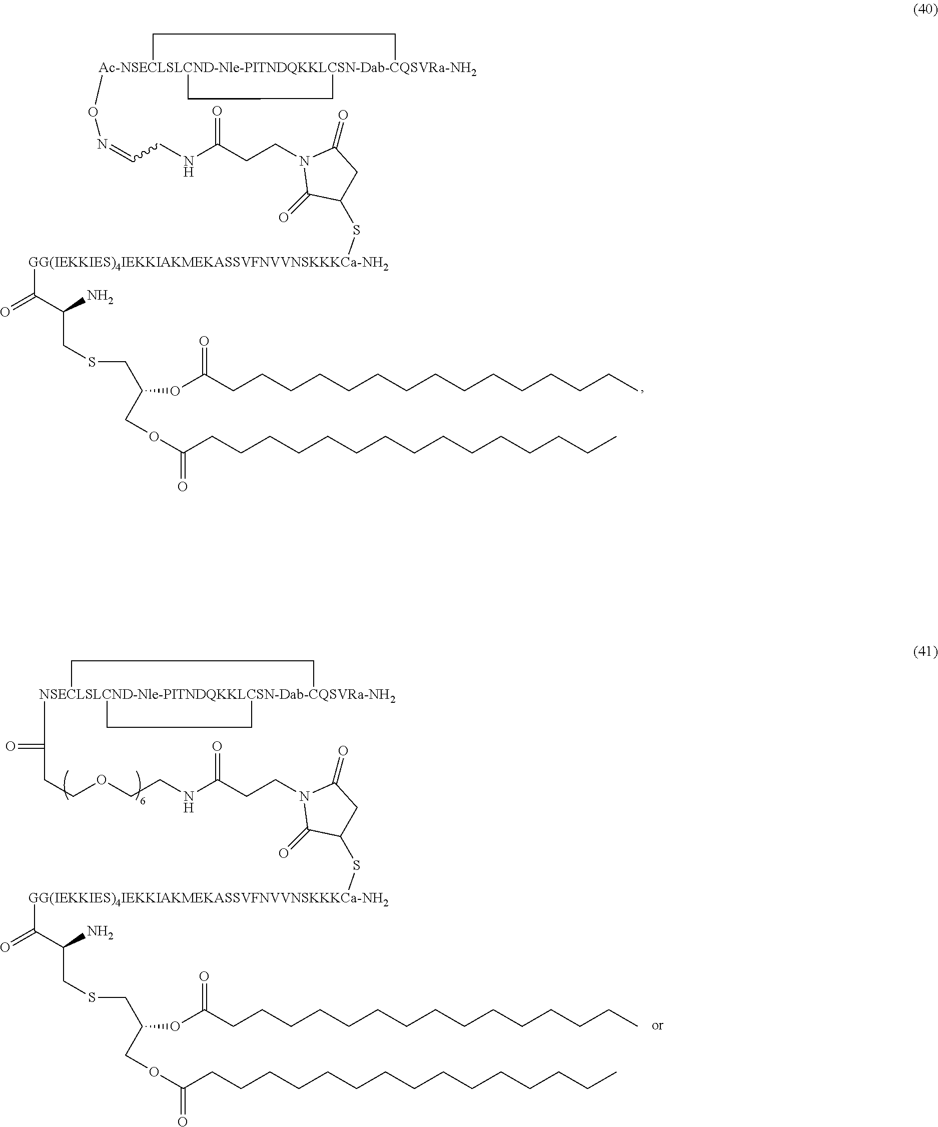

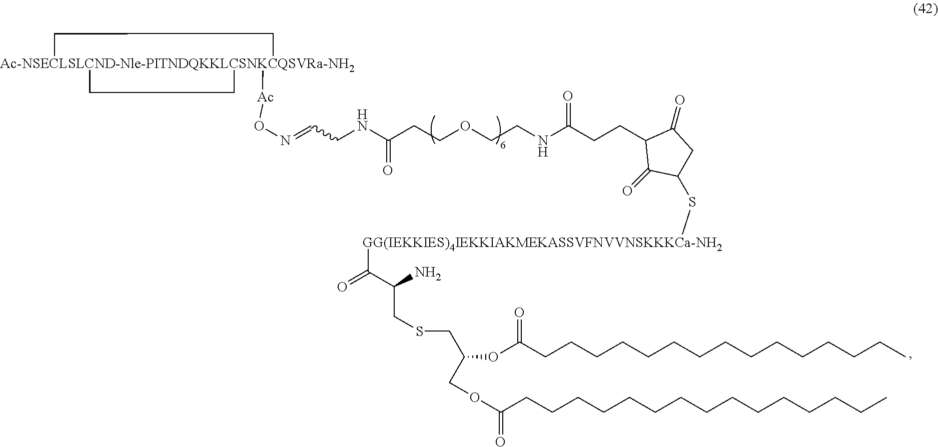

In a preferred embodiment, a bundle of conjugates is assembled from 2, 3, 4, 5, 6 or 7 conjugates. Preferably said bundle comprises 2, 3, 4 or 5 of the conjugates, more preferably 3 of the conjugates. In a preferred embodiment, said bundle of conjugates comprises 2, 3, 4, 5, 6 or 7 conjugates, wherein said conjugate is selected from any one of the formula (38), (39), (40), (41) or (42), wherein further preferably said conjugate is formula (38). In a more preferred embodiment, said bundle of conjugates comprises 3 conjugates, wherein said conjugate is of formula (38). The inventors have shown that said SVLP exposing an RSV-F protein, a variant or a fragment induces the generation of neutralizing antibodies directed against the epitope recognized by the palivizumab, said SVLP being administered by epicutaneous application.

In a specific aspect, the invention relates to a synthetic virus-like-particle for use in a method of prevention of a disease caused by RSV by epicutaneous vaccination with said particle, wherein said synthetic virus-like-particle comprises
a peptide chain comprising a coiled coil-domain selected from the group consisting of sequences depicted in SEQ ID NO: 6, 7, 8, 9, 10, 11, 12, 13, 14, 15, 16, 17, 18, 19, 20, 21, 22, 23, 24, 25, 26, 27, 28, 29, 30 and variants thereof,
the lipid moiety 2,3-dipalmitoyl-S-glycerylcysteine,
the amino acid sequence depicted in SEQ ID NO:2.

All the technical data mentioned herein are applicable.

Preferably, said synthetic virus-like particle comprising at least one bundle of conjugates. In a very preferred embodiment, said synthetic virus-like particle comprising at least one bundle of conjugates of the present invention, wherein said conjugate is selected from any one of the formula (38), (39), (40), (41), or (42), wherein preferably said conjugate is selected from any one of the formula (38), (40), (41), or (42), and wherein further and most preferably said conjugate is formula (38).

According to a preferred embodiment, in said bundle, the coiled coil peptide chain segments of said peptide moieties comprised by said conjugates are coiled together, preferably said coiled coil peptide chain segments are helically coiled together, more preferably said coiled coil peptide chain segments are alpha-helically coiled together. In a preferred embodiment, said coiled coil peptide chain segments of said peptide moieties are coiled together left-handed or right-handed. According a preferred embodiment, in said bundle, said coiled coil peptide chain segments of said peptide moieties form an alpha-helical left-handed coil.

In a preferred embodiment, said coiled coil peptide chain segments have a parallel orientation, i.e. they run in the same direction; or they have an anti-parallel orientation, i.e. they run in directions opposite to each other; wherein the first option is preferred. The term “direction” is based on the direction of a peptide chain having on one side an N-terminus and on the other side a C-terminus. In a preferred embodiment of said inventive bundle, said coiled coil peptide chain segments of said peptide moieties form a left-handed alpha-helical coiled coil, wherein the coiled coil peptide chain segments have a parallel orientation in said coiled coil. Preferably, said bundle comprises 2 to 7 (e.g. dimer, trimer, tetramer, pentamer, hexamer or heptamer), more preferably 2, 3, 4 or 5, again more preferably 3 helically twisted coiled coil peptide chain segments, having a parallel orientation in said coiled coil.

In a preferred embodiment, the SVLP of the invention used for vaccination comprises a variant of an RSV-F protein, wherein said variant is a cyclic peptide comprising an amino acid sequence (I), wherein said amino acid sequence (I) comprises, preferably consists of SEQ ID NO: 44. In another very preferred embodiment, the amino acid sequence (I) comprises, preferably consists of a sequence selected from any one of SEQ ID NO: 45-88, preferably SEQ ID NO: 45-83, more preferably SEQ ID NO: 45-64, again more preferably SEQ ID NO: 45-51. In another embodiment, said SVLP comprises at least one bundle of conjugates of the present invention, wherein said conjugate is selected from any one of the formula (38), (39), (40), (41), or (42), wherein preferably said conjugate is selected from any one of the formula (38), (40), (41), or (42), and wherein further preferably said conjugate is formula (38).

In another preferred embodiment, said SVLP of the invention used for vaccination comprises a fragment of an RSV-F protein, preferably of a RSV-F protein of SEQ ID NO:1. Preferably said fragment comprises, preferably consists of a sequence selected from SEQ ID NO: 2-5. In another embodiment, said fragment comprises, preferably consists of sequence SEQ ID NO: 5. In another preferred embodiment, said SVLP of the invention used for prime/boost vaccination comprises at least one bundle of conjugates of the present invention, wherein said conjugate comprises (a) a lipopeptide building block selected from LBB-1 to LBB6 and (b) an RSV-F protein fragment selected from SEQ ID NO: 2-5 or SEQ ID NO: 5, directly coupled or coupled via a linker to the lipopeptide block.

As explained above, for use in the present invention, the particle of the invention, preferably the SVLP of the invention, is applied epicutaneously to a skin area of the subject. The expression “epicutaneous application” indicates an application on skin surface, using an application device and under conditions allowing a contact with the surface of the skin. Skin application should be maintained for a period of time sufficient to allow penetration of an antigen in the superficial layer(s) of the skin and/or contact of the antigen with immune cells.

Epicutaneous application is preferably performed using a device suitable to maintain contact between the particles of the invention and the skin of the subject. Such devices include, without limitation, a patch, a tape, a dressing, a sheet, or any other form known to those skilled in the art. Preferably, the skin device is a patch, even more preferably an occlusive patch. In the most preferred embodiment, the method of the invention uses a skin patch device as described by the applicant in e.g., WO2011/128430; WO02/071950, or WO2007/12226. These skin patch devices are referred to as Viaskin. Such application can effectively lead to antigen exposure of the treated subject, and to the generation of suitable immune response. More precisely, the inventors have shown that the use of such a device conferred effective anti-RSV protective immunity in vivo, with the generation of neutralizing antibodies directed against RSV-F, hence suitable for protecting infants during very early stage of life.

In a specific embodiment, such a device is occlusive and is configured to use the particles of the invention in dry form. In some embodiments, the particles may be maintained on the patch through electrostatic and/or Van der Waals forces, with no added adhesive. The preparation and characteristics of such a device (termed Viaskin) are disclosed in detail in the above quoted applications.

As used herein, “an electrostatic patch” refers to a patch wherein the antigen, preferably, the particle of the invention adheres to the skin facing side of the patch though electrostatic forces, without the mean of any adhesive.

In a particular embodiment, the portion of the backing of the patch bearing the particle of the invention is not in direct contact with the skin. In this embodiment, the patch contains a “so-called” condensation chamber. The height of the chamber defined by the backing, the periphery of the backing and the skin is in the range of 0.1 mm to 1 mm.

For the performance of the present invention, it is particularly suited to use a device comprising a backing adapted to create with the skin a hermetically closed chamber, this backing having on its skin facing side within the chamber the dry particle of the invention adhered through electrostatic forces and/or Van der Waals forces. Upon application to the skin, moisture increases in the chamber, leading to the dissolution of the dry particle of the invention and contacting with the skin.

In another preferred embodiment of the invention, the particle, preferably the SVLP of the invention is applied on the skin of the mammalian using an occlusive patch device comprising a support to which the particles are bound. Preferably, the particle is bound to the support of the patch through electrostatic or Van der Waals forces, with no added adhesive. In particular embodiments, the support of the patch may be comprised of glass or polymer chosen from the group consisting of cellulose plastics (CA, CP), polyvinyl chloride (PVC), polypropylenes, polystyrenes, polyurethanes, polycarbonates, polyacrylics in particular poly(methyl methacrylate (PMMA), polyolefines, polyesters, polyethylenes (PE), polyethylene terephthalate (PET), fluoropolymers (PTFE for example) and ethylene vinyl acrylates (EVA).

For instance, the occlusive skin patch may comprise a breathable overadhesive, a backing preferably made of polyethylene terephthalate (PET) and an adhesive crown, the adhesive crown and the backing being adapted to create with the skin a hermetically closed chamber. Typically, the antigen is present in dry form onto the skin facing side of the backing. The antigen may have been deposed onto the backing by spray drying for instance by means of electrospray as described in WO2009095591.

The particle of the invention can be present in pure form or as an admixture with pharmaceutically acceptable excipient(s), other antigen/allergen(s) and/or with adjuvant(s) in the patch.

The epicutaneous vaccination is typically performed on a healthy skin area of the subject.

In one embodiment, said epicutaneous vaccination is performed by the application of said skin patch device on an intact area of the skin, i.e., with no abrasive or perforating pre-treatment of the skin.

In another embodiment, said epicutaneous vaccination is performed by the application of said skin patch device on a pretreated skin.

As used herein, the pretreatment of the skin on the application site of the patch encompasses treatments aiming at promoting the penetration of the antigens (namely the particle of the invention) in the upper layer of the skins, preferably in the epidermis. The pretreatment of the skin is preferably contemplated when the subject is an adult. The pretreatment of the skin is preferably superficial. Typically, the skin pretreatment can alter one or several epidermis cell layers while maintaining the integrity of dermis and the hypodermis of the skin. Preferably, the skin pretreatment does not alter the epidermis basal layer as well. Skin treatments encompass, without being limited to, skin microporation, such as laser microporation, skin cleansing, gentle skin stripping, skin exfoliation, and the like.

When the subject is an infant or a child under the age of 12 years, skin pretreatment is avoided.

Application of the patch on the skin is typically performed under conditions and/or for a period of time sufficient to allow the antigen to penetrate into the stratum corneum of the epidermis and/or to reach immune cells. The duration of the patch application on the skin is preferably from 2 to 96 hours, such as from 5 h to 72 h. For an occlusive patch, such as a Viaskin, the duration of contact is typically from 2 hours to 48 hours, such as from 2 h to 6 h, from 6 h to 12 h, from 12 h to 24 h, from 24 h to 36 h and from 36 h to 48 h. The duration of skin patch application on skin can depend on the immunotherapeutic effect which is sought, e.g. the induction of an immune response or the enhancement of a pre-existing response. For instance, the patch can be applied on the skin during about 24 h. The application of the skin patch comprising the antigen can be single or repeated, typically 1 to 5 times such as 2, 3 and 4 times. Preferably, two consecutive applications may be separated by 1 day to several weeks, typically by 1 week to 10 weeks. In a particular embodiment, the method of the invention comprises from 1 to 5 patch applications of 6, 24, 36 or 48 hours, two consecutive applications being separated by at least 1 week. In a specific embodiment, the application of the patch of the invention is repeated at least 2 times, for instance 2, or 3 times, at 2-, 3-, or 4-week interval.

When using the particle of the invention comprising an RSV-F protein, a variant or a fragment thereof in a method of prevention of a disease caused by RSV by epicutaneous vaccination preferably an immunologically effective amount of the particle, preferably the synthetic virus like particle of the present invention is administered. As used herein, the term “effective amount” refers to an amount necessary or sufficient to realize a desired biologic effect. Preferably, the term “effective amount” refers to an amount of the particle of the present invention that is capable to (i) treat or prevent the particular disease, medical condition, or disorder, (ii) attenuate, ameliorate, or eliminate one or more symptoms of the particular disease, medical condition, or disorder, or (iii) prevent or delay the onset of one or more symptoms of the particular disease, medical condition, or disorder caused directly or indirectly by RSV. An immunogenically effective amount, as herein understood, is an amount that is capable of modulating, preferably enhancing the response of the immune system of a subject to an antigen or pathogen.

The invention further relates to the particle of the invention for use in a method for eliciting or modulating an immune response against RSV or a method of limiting the risk of developing a disease caused by RSV, preferably an infection caused by RSV, wherein an immunogenically effective amount of the particle of the invention is administered to a subject, preferably a human, more preferably a child or pregnant female, by epicutaneous vaccination with said particle.

The invention further relates to a method of vaccination against RSV an RSV infection, or a method for eliciting or modulating an immune response against RSV, or a method of limiting the risk of developing a disease caused by RSV, preferably an infection caused by RSV, wherein an immunogenically effective amount of the particle of the invention is administered to a subject, preferably a human, more preferably a child or a pregnant female, by epicutaneous vaccination with said particle.

The inventors have further shown that deposition of the particle according to the invention on the surface of such patches as Viaskin can be performed e.g., using electrospray process, without denaturizing the particle. Therefore, in a third aspect, the invention also resides in a method for preparing such a skin patch device comprising depositing, preferably be electrospraying, at least one particle of the invention comprising an RSV-F protein, a variant or a fragment thereof on a surface of a skin patch device. Preferably, said variant of RSV-F is a cyclic peptide comprising an amino acid sequence (I), wherein said amino acid sequence (I) comprises, preferably consists of SEQ ID NO: 44, preferably said SEQ ID NO: 44 is a sequence selected from the group consisting of SEQ ID NO: 45-88, preferably SEQ ID NO: 45-88, more preferably 45-64, again more preferably 45-51.

The dose of antigen used in the invention may be adjusted by the skilled artisan. However, typically a dose of 10 μg-10 mg is used for each application.

In a further embodiment, the particle is not used with an external adjuvant, i.e. an adjuvant which is added to the particle of the invention, on the surface of the skin patch device of the invention or on the surface of the skin area where the skin patch device will be applied and which is meant to be administered simultaneously with the particles according to the invention. As used herein the term “adjuvant” refers to a compound that, when used in combination with a specific immunogen (e.g. a particle comprising a RSV F protein, a variant or a fragment thereof) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

In a fourth aspect, the invention relates to a skin patch device comprising an application surface, wherein the application surface contains particles of the invention comprising an RSV-F protein, a variant or a fragment thereof. In preferred embodiment, said skin patch devices comprise SVLP exposing the antigenic site II of the RSV-F protein or comprising a variant of RSV-F, wherein said variant is a cyclic peptide comprising an amino acid sequence (I), wherein said amino acid sequence (I) comprises, preferably consists of SEQ ID NO: 44, preferably said SEQ ID NO: 44 is a sequence selected from the group consisting of SEQ ID NO: 45-88, preferably SEQ ID NO: 45-83, more preferably 45-64, again more preferably 45-51, again more preferably SEQ ID NO: 45.

In a further embodiment, said SVLP present in the skin patch device, comprises at least one, and even consists of, bundles of conjugates of the present invention, wherein said conjugate is selected from any one of the formula (38), (39), (40), (41), or (42) and combinations thereof, preferably the conjugate of formula (38).

In a particular embodiment, the SVLP present in the skin patch consists in the self-assembly of bundles made of three conjugates selected from any one of the formula (38), (39), (40), (41), or (42) and combinations thereof, preferably the conjugate of formula (38). As described above, the SVLP has preferably a mean diameter of less 50 nm, e.g. from 10 nm to 40 nm or from 20 nm to 30 nm.

An example of such a skin patch device is Viaskin-SVLP-FsII as used in the experimental section.

All the previously disclosed technical data are applicable here.

In a fifth aspect, the invention also relates to method of vaccination against RSV infection, said method comprising the epicutaneous application of a particle comprising a RSV-F protein, a variant or a fragment thereof. The invention further relates to a method for vaccinating infants against RSV, comprising maternal epicutaneous vaccination by repeated application of a skin patch device containing a particle comprising a RSV-F protein, a variant or a fragment thereof to an area of the skin of the mother during pregnancy.

In a sixth aspect, the invention relates to the use of a particle comprising an RSV-F protein, a variant or a fragment thereof in the manufacture of a drug for the prevention of a disease caused by RSV, wherein the drug is delivered by means of a skin patch by epicutaneous route to provide vaccination against RSV. The invention also relates to the use of a particle comprising an RSV-F protein, a variant or a fragment thereof in the manufacture of a drug for passively vaccinating an infant against RSV, wherein the drug is administered by means of a skin patch by epicutaneous route to the infant's mother.

All the previously disclosed technical data are applicable here to these aspects of the invention.

Not only are the disclosure of WO 2020/127728 A1, WO 2018/229156 A1, WO 2015/082501 A1, WO 2008/068017 A1, incorporated herein in their entirety by way of reference, but all the disclosures of WO 2020/127728 A1, WO 2018/229156 A1, WO 2015/082501 A1, WO 2008/068017 A1, in particular, the disclosure related to the specific linking, coupling, attaching and connecting moieties/residues, spacers, lipid and peptide moieties, lipopeptide building blocks, conjugates and other components and moieties, and the generated biological data hereto are specifically incorporated herein in its entirety by way of reference.

Further aspects and advantages of the invention will be disclosed in the following illustrative experimental section.

### LEGEND TO THE FIGURES

FIG. 1: Evaluation of the efficacy of Viaskin-SVLP-FsII as a boost epicutaneous vaccine. Mice were primed with a subcutaneous injection of 150 μg of SVLP-FsII, (also called herein V-306 SVLP (groups 2 to 5). Three weeks later, mice were boosted with a single application of Viaskin patch loaded with 100 or 200 μg of SVLP-FsII, for 48 hours (groups 3 and 4). As a negative control for boost immunization, mice received a single application of Viaskin patch loaded with excipient alone (PBS 1×), for 48 hours (group 2). As a positive control for boost immunization, mice received an injection of 150 μg of V-306 SVLP-FsII, namely V-306 SVLP, by subcutaneous route (group 5). As a negative control for prime and boost immunizations, mice received two application of Viaskin patch loaded with excipient alone (PBS 1×), for 48 hours, at day 0 and day 21 (group 1).

FIG. 2: Measurement of anti-FsII antibodies in mouse sera and BAL by FsII-specific ELISA. Mice were immunized as described in FIG. 1. Blood samples were collected three weeks after the prime immunization (day 21) and two weeks after the boost immunization (day 35) to prepare sera. At day 35, mice were sacrificed and bronchoalveolar lavages (BAL) were collected. Anti-FsII antibody titers were measured by ELISA from sera (A) and BAL (B) using FsII peptide as a coating antigen and mouse anti IgG (H+L) secondary antibody. Data are median±interquartile range of individual IgG titers (n=10 per experimental group). P values were determined using the Man-Whitney non-parametric test (*, P<0.05; **, P<0.01; ***, P<0.001; ****, P<0.0001; n.s., non-significant).

FIG. 3: Measurement of neutralizing and palivizumab-competitive antibody (PCA) titers in sera. Mice were immunized as described in FIG. 1. (A) Neutralizing antibody titers were measured from heat inactivated sera (30 min at 56° C.) collected at day 35. Briefly, neutralization assay was performed using Hep2 cells on microplates and using RSV A Memphis strain. Neutralizing antibody titers were determined by the Reed and Muench method as the reciprocal of the highest dilution of each serum, which suppressed cyto-pathogen effect. (B1) PCA titers were measured from sera collected at day 35 by competitive ELISA. Briefly, serial dilutions of sera were mixed 1:1 with a fixed concentration of Palivizumab. This mixture was then added to an ELISA plate coated with FsII peptide. Residual biding of Palivizumab was revealed using a peroxidase-conjugated anti-human IgG and TMB colorimetric substrate. Optical density at 450 nm (B1) was plotted against mouse serum dilution. Non-linear curve fit was performed using Boltzmann sigmoidal equation. PCA titers (B2) were defined as the reciprocal dilution of sera that inhibit 50% of the optical density measured at 450 nm. The limit of detection was indicated with a dotted line. Data are median±interquartile range of individual titers (n=6-10 per experimental group). P values were determined using the Man-Whitney non-parametric test (**, P<0.01; * P<0.001; ****, P<0.0001; n.s., non-significant).

FIG. 4: Evaluation of the capacity of Viaskin-SVLP-FsII boost epicutaneous vaccine to enhance protection against RSV infection. Mice were primed with a subcutaneous injection of 150 μg of SVLP-FsII (also called herein V-306 SVLP) (groups 2 to 4). Three weeks later, mice were boosted with a single application of Viaskin patch loaded with 150 μg of SVLP-FsII, preferably V-306 SVLP, for 48 hours (group 3). As a negative control for boost immunization, mice received a single application of Viaskin patch loaded with excipient alone (PBS 1×), for 48 hours (group 2). As a positive control for boost immunization, mice received an injection of 150 μg of SVLP-FsII, by subcutaneous route (group 4). As a negative control for prime and boost immunizations, mice received an injection of excipient (PBS 1×) by subcutaneous route at day 0 and an application of Viaskin patch loaded with excipient alone (PBS 1×), for 48 hours, at day 21 (group 1). As a positive control for viral protection, mice were infected intranasally at day 0 with RSV A2. As a control for vaccine-induced immunopathology, mice were primed and boosted at day 0 and day 21 by an injection of formalin-inactivated RSV by intramuscular route (group 6). A blood sample was collected three weeks after the boost immunization (day 42) and mice were challenged intranasally with 1×10⁶ plaque-forming units (pfu) of RSV A2 strain. Mice were sacrificed at 5 days post-infection and lungs were collected to measure pulmonary viral load, perform histological analysis and to extract mRNA.

FIG. 5: Measurement of F-specific I2G1/I2G2a and RSV-neutralizing antibodies in vaccinated mice and pulmonary viral load following RSV challenge. Mice were immunized as described in FIG. 4. Blood samples were collected three weeks after the boost immunization (day 42) to prepare sera. (A) Anti-F antibody titers were measured by ELISA from sera using F protein as a coating antigen and mouse anti-IgG1 (left panel) or anti-IgG2a (right panel) secondary antibodies. Data are median±interquartile range of individual IgG titers (n=10 per experimental group). (B) Neutralizing antibody titers were measured by plate reduction assay from heat inactivated sera (30 min at 56° C.) collected at day 35. (C) Palivizumab-competitive antibody titers were evaluated as described in FIG. 3. (D) Mice were infected at day 42 and were sacrificed at day 5 post-infection. Lungs were collected and homogenized to measure viral load by plate titration. Viral titers were normalized to the weight of individual lungs to obtain a viral concentration expressed as the number of plaque-forming units (pfu) per gram of lung. Data are mean+SEM of individual data (n=4-10 per experimental group). P values were determined using the Man-Whitney non-parametric test (*, P<0.05; **, P<0.01; ***, P<0.001; ****, P<0.0001; n.s., non-significant). For D panel, the significance measured between the group 1 (non-vaccinated group) and each of other groups is indicated above each histogram.

FIG. 6: Evaluation of lung pathology in vaccinated mice following RSV challenge. Mice were immunized and challenged as described in FIG. 4. Lungs were collected at day 5 post infection and fixed using formalin. Fixed lungs were embedded in paraffin and histological sections were performed. Lung sections were colored using Hematoxylin Eosin Safran (HES) staining and analyzed. Four pathology criteria were evaluated (alveolitis, interstitial pneumonia, perivasculitis, peribronchiolitis) and a pathology score ranging from 0 to 100 was given to each sample. (A) Data are median+interquartile range of individual data (n=4-10 per experimental group). (B) A representative photograph of lung sections was shown for groups 2, 3, 4, 5, as indicated.

FIG. 7: Measurement of pulmonary cytokine secretion in vaccinated mice following RSV challenge. Mice were immunized and challenged as described in FIG. 4. Lungs were collected at day 5 post infection and messenger RNA (mRNA) was extracted from lung tissues. The quantity of IL-5, IL-13, IFN-γ and IL-2 transcripts was measured by quantitative RT-PCR using specific primers. Data are mean+SEM of individual data and are expressed as relative mRNA level (n=4-10 per experimental group). The significance measured between the non-infected group and each of other groups is indicated above each histogram.

FIG. 8. Structural characterization. A, The sequences of peptides FsIIm (SEQ ID NO: 130), V-306p (Nle=L-norleucine, Dab=L-diaminobutyric acid, D-Ala=D-alanine), the synthetic lipopeptide (shown in single amino acid letter code), with Pam2Cys, the coiled coil heptad repeat IEKKIES that forms a trimeric helical bundle, with T helper epitope underlined, and C-terminal KKKCa, (a=D-alanine) and, V-306. Pam2Cys is S—[R-2,3-bis(palmitoyloxy)propyl]-R-cysteine. B, Left, Solution NMR structures of FsIIm. A superimposition of the final 20 structures is shown. Right, Superimposition of one typical NMR structure of FsIIm and the Motavizumab antigen from PDB file 3IXT. C, Schematic representation of the components and assembly of a SVLP: SVLPs, such as V-306 SVLP, are formed by aggregation of multimeric conjugate bundles, here trimeric conjugate bundles comprising three conjugates, such as V-306 (ball=epitope mimetic, such as V-306p, cylinder=helical coiled-coil domain, wavy line=lipid moiety, such as Pam2Cys lipid) resulting in ca. 25-30 nm diameter micelle-like nanoparticles.

FIG. 9. Synthesis of V-306. The synthetic route to conjugate V-306.

FIG. 10. EM and DLS data for V-306. A, Negative staining transmission electron micrograph of SVLPs formed by the V-306 lipopeptide dissolved in tris(hydroxymethyl)amino-methane (Tris) buffer containing 0.9% NaCl, pH 7.4. Scale bar 5×100 nm. B, V-306 lipopeptide was dissolved in PBS (0.5 mg/mL) at pH 7.4, and analyzed in a Wyatt DynaPro DLS instrument at 25° C. Size distributions are shown by regularization as intensity distributions. The average radius, % polydispersity and polydispersity index (PDI) are indicated.

### EXAMPLES

### Example 1: Evaluation of the Efficacy of Viaskin-SVLP-FsII as an Epicutaneous Boost Vaccine for the Induction of Specific RSV-F Humoral Response and the Protection Against RSV Infection

The inventors aim to evaluate the capacity of Viaskin patch loaded with SVLP-FsII to induce an RSV-F specific humoral immunity when administered epicutaneously as a boost vaccine. Two experiments were designed requiring 50 and 60 BALB/c mice respectively. The first experiment was performed at DBV Technologies (Montrouge, France) and the second experiment was performed at Sigmovir Biosystems Inc. (Rockville, USA).

Study Design

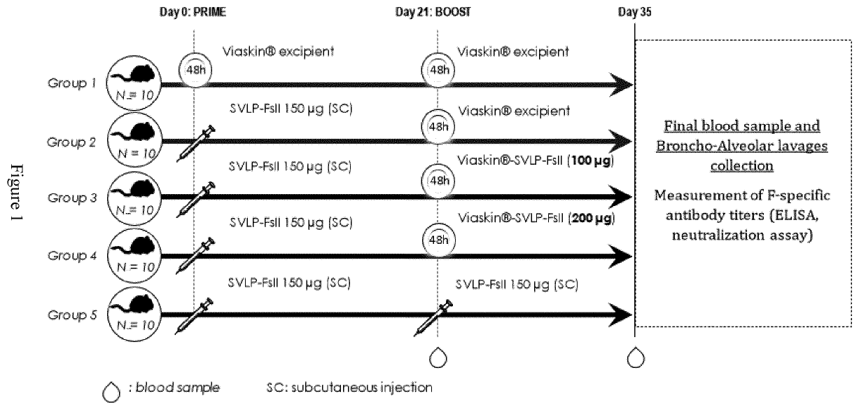

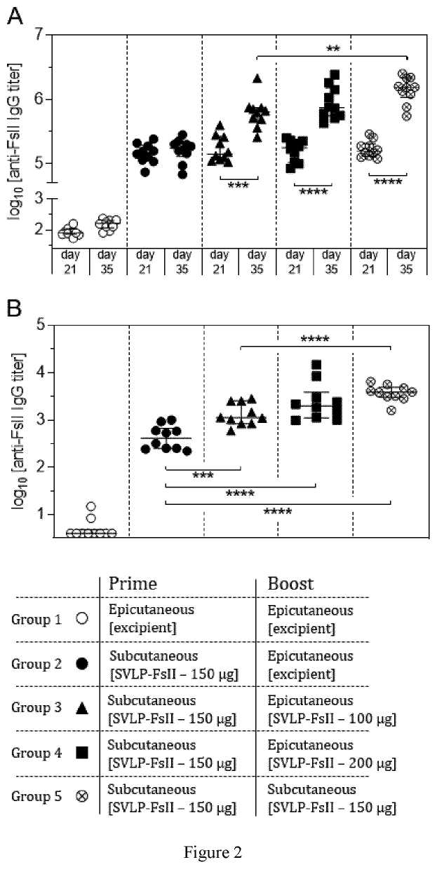

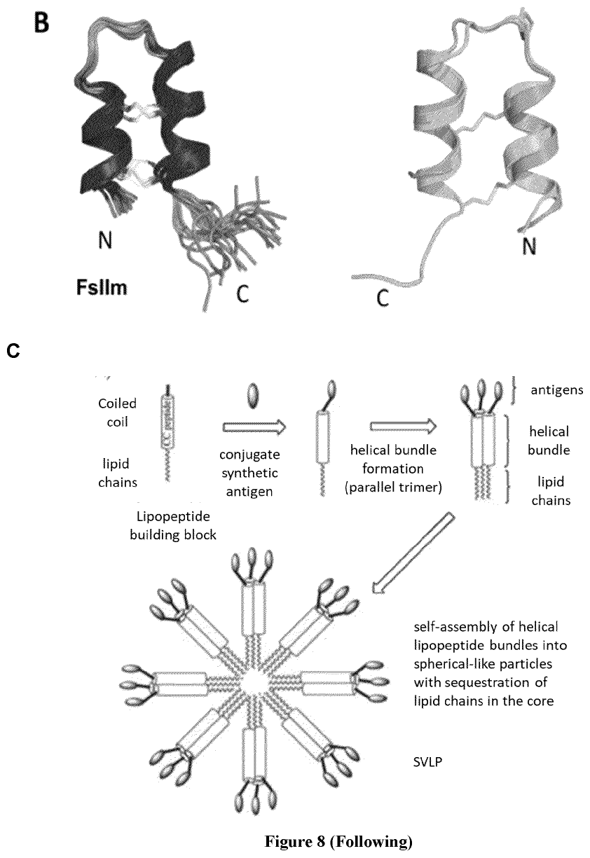

SVLP-FsII used in Example 1 and FIGS. 1-7 is V-306 SVLP (cf. FIG. 8 and description thereof), which is an SVLP comprising trimeric bundles of conjugates of formula (38) and which is formed by self-aggregation of conjugates of formula (38) into trimeric bundles that self-assemble further into SVLPs. Conjugate of formula (38) comprises the mimetic of SEQ ID NO: 85 (i.e. SEQ ID NO: 45 with an N terminal AOAc residue), herein also called V-306p. Each of said trimeric bundles of conjugates included in V-306 SVLP consists of 3 conjugates of formula (38).

For the first experiment (FIG. 1), mice were primed at day 0 with SVLP-FsII, namely V-306 SVLP (150 μg) by subcutaneous route (groups 2 to 5). Three weeks later (day 21), mice were boosted with a single application of Viaskin-SVLP-FsII patch (100 or 200 μg of SVLP-FsII, namely V-306 SVLP, per patch groups 3 and 4, respectively). As a negative control for boost immunization, mice received a Viaskin patch loaded with excipient alone (PBS 1×) at day 21 (group 2). As a positive control, mice received a boost injection of SVLP-FsII (150 μg SVLP V-306) by subcutaneous route at day 21 (group 5). As a negative control for prime and boost immunizations, mice received two Viaskin patches loaded with excipient alone (PBS 1×) at day 0 and day 21 (group 1). A blood sample was collected at day 21, before boost immunization. Two weeks after the boost immunization, mice were sacrificed and a final blood sample and bronchoalveolar lavages were collected for the evaluation of FsII specific humoral responses (FsII-specific ELISA and palivizumab competition assay) and for the measurement of RSV-neutralizing antibodies.

For the second experiment (FIG. 4), mice were primed at day 0 with SVLP-FsII (SVLP V-306 150 μg) by subcutaneous route (groups 2 to 4). Three weeks later (day 21), mice were boosted with a single application of Viaskin-SVLP-FsII patch (150 μg of SVLP-FsII, namely SVLP V-306 per patch, group 3). As a negative control for boost immunization, mice received a Viaskin patch loaded with excipient alone (PBS 1×) at day 21 (group 2). As a positive control, mice received a boost injection of SVLP-FsII (150 μg SVLP V-306) by subcutaneous route at day 21 (group 4). As a negative control for prime and boost immunizations, mice received an injection of excipient (PBS 1×) by subcutaneous route at day 0 and a Viaskin patch loaded with excipient alone (PBS 1×) at day 21 (group 1). As a model of vaccine-induced immunopathology, mice were primed at day 0 and boosted at day 21 with formalin-inactivated RSV (group 5) and, as a positive control for protection, mice were infected intranasally at day 0 with RSV A2 (group 6). A single blood sample was collected three weeks after the boost immunization (day 42) for the evaluation of F-specific humoral responses (F-specific ELISA [IgG1 and IgG2a] and palivizumab competition assay) and for the measurement of RSV-neutralizing antibodies. Following blood sample, all mice were challenged intranasally with RSV A2 strain (1×10⁶ pfu per mouse). At day 5 post-infection, mice were sacrificed, and lungs were collected for the measurement of viral load, histological analysis and mRNA extraction for qPCR on IL-5, IL-13, IFN-γ and IL-2 transcripts.

Results

1. Viaskin-SVLP-FsII is Able to Systemically and Locally Boost Anti-F Antibody Responses Mice were immunized as described in FIG. 1. Sera samples were collected from individual mice three weeks after le prime immunization (day 21) and two weeks after the boost immunization (day 35). From these sera, FsII-specific antibody titers were measured by ELISA (FIG. 2).

In mice previously primed subcutaneously with SVLP-FsII, epicutaneous boost immunization with Viaskin-SVLP-FsII induced a strong and a significant increase of FsII specific antibody titers compared to the mice that received Viaskin-excipient patch (FIG. 2A) (increase of 0.6 to 0.7 Log 10 between day 21 and day 35, based on median values; mean antibody titer of 5.8±0.2 log 10 for Viaskin-SVLP-FsII 100 μg at day 35, p<0.001 compared to day 21; mean antibody titer of 5.9±0.2 for Viaskin-SVLP-FsII 200 μg at day 35, p<0.0001 compared to day 21). This boost effect was in the same range than that observed after a subcutaneous boost immunization with SVLP-FsII (mean antibody titer of 6.1±0.2 log 10 at day 35, p<0.0001 compared to day 21, p<0.01 compared to Viaskin-SVLP-FsII 100 μg at day 35 and non-significant compared to Viaskin-SVLP-FsII 200 μg at day 35). The dose effect observed between Viaskin-SVLP-FsII 100 μg and Viaskin-SVLP-FsII 200 μg was in favor of the highest dose.

To evaluate the capacity of Viaskin-SVLP-FsII to boost local antibody response in lungs, bronchoalveolar lavages (BAL) were collected at day 35. Anti-FsII antibody response was measured by ELISA (FIG. 2B). Epicutaneous boost immunization with Viaskin-SVLP-FsII induced a significant increase of FsII specific IgG titers in BAL compared to the mice that received Viaskin-excipient patch (mean antibody titer of 3.1±0.2 log 10 for Viaskin-SVLP-FsII 100 μg, p<0.001 compared to the Viaskin-excipient group; mean antibody titer of 3.4±0.4 for Viaskin-SVLP-FsII 200 μg, p<0.0001 compared to the Viaskin-excipient group). The boost effect observed with Viaskin-SVLP-FsII 200 μg was in the same range than that observed after a subcutaneous boost immunization with SVLP-FsII (mean antibody titer of 3.6±0.2 log 10, p<0.0001 compared to the Viaskin-excipient group, p<0.0001 compared to Viaskin-SVLP-FsII 100 μg and non-significant compared to Viaskin-SVLP-FsII 200 μg). Again, the dose effect observed between Viaskin-SVLP-FsII 100 μg and Viaskin-SVLP-FsII 200 μg was in favor of the highest dose but not significant.

To conclude, these results demonstrate that epicutaneous boost immunization with Viaskin-SVLP-FsII is able to significantly increase FsII-specific antibody response in mice primed by subcutaneous route.

2. Epicutaneous Boost Immunization with Viaskin-SVLP-FsII Increases the Level of RSV-Neutralizing Antibodies and the Level of Palivizumab-Competitive Antibodies (PCA) in Mice

To evaluate the capacity of antibodies induced by epicutaneous boost immunization with Viaskin-SVLP-FsII to neutralize RSV infectivity in vitro, sera samples were sent to hVIVO services LTD (London, UK) for microneutralization assays. RSV microneutralization assay were performed on Hep2 cells using RSV A Memphis strain.

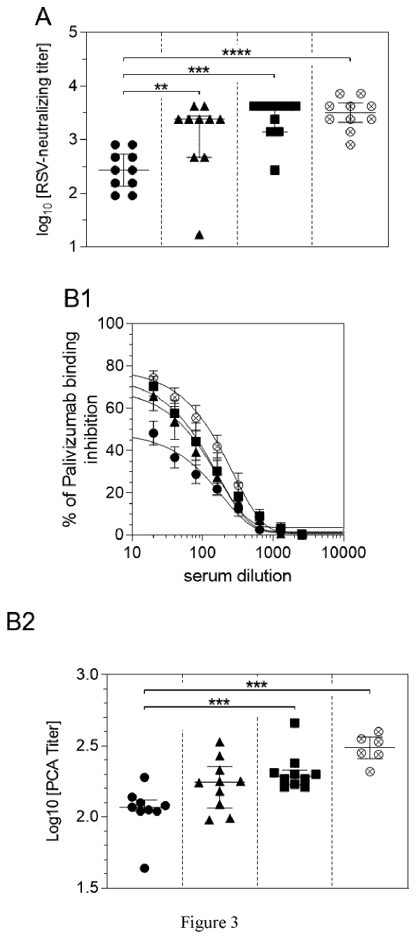

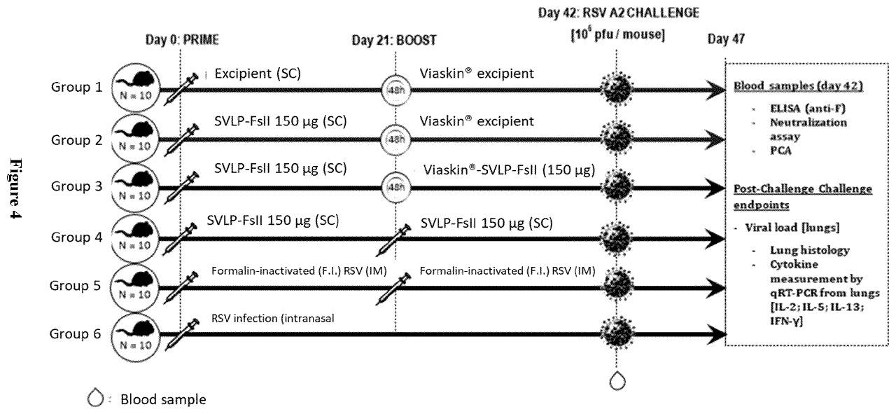

In mice previously primed subcutaneously with SVLP-FsII, epicutaneous boost immunization with Viaskin-SVLP-FsII induced a significant increase of RSV-neutralizing antibody titers compared to the mice that received Viaskin-excipient patch (FIG. 3A) (mean neutralizing antibody titer of 3.1±0.7 log 10 for Viaskin-SVLP-FsII 100 μg, p<0.01 compared to the Viaskin-excipient group; mean antibody titer of 3.4±0.4 for Viaskin-SVLP-FsII 200 μg, p<0.001 compared to the Viaskin-excipient group). The boost effect observed with Viaskin-SVLP-FsII was similar to that observed after the subcutaneous boost immunization with SVLP-FsII (mean antibody titer of 3.5±0.3 log 10, p<0.0001 compared to the Viaskin-excipient group, non-significant compared to Viaskin-SVLP-FsII (100 or 200 μg). As it was assessed by ELISA, the dose effect observed between Viaskin-SVLP-FsII 100 μg and Viaskin-SVLP-FsII 200 μg was in the favour of the highest.

To go further and to evaluate the capacity of the antibodies induced by epicutaneous boost immunization with Viaskin-SVLP-FsII to compete with Palivizumab binding to FsII peptide, a Palivizumab-competitive ELISA was set up (FIG. 3B). In agreement with neutralization results, epicutaneous boost immunization Viaskin-SVLP-FsII induced an increase of Palivizumab-competitive antibodies (PCA) as compared to the mice that received Viaskin-excipient patch (mean PCA titer of 2.2±0.2 log 10 for Viaskin-SVLP-FsII 100 μg, non-significant compared to the Viaskin-excipient group; mean PCA titer of 2.3±0.1 log 10 for Viaskin-SVLP-FsII 200 μg, p<0.001 compared to the Viaskin-excipient group).

To conclude, these results demonstrated that epicutaneous boost immunization with Viaskin-SVLP-FsII is able to significantly raise the level of high-quality and functional antibodies in mice.

3. Epicutaneous Boost Immunization with Viaskin-SVLP-FsII Increases the Level of F-Specific Neutralizing Antibodies and Protects Animal Against RSV Infection

To confirm previous results and to evaluate the capacity of epicutaneous boost immunization with Viaskin-SVLP-FsII to increase protection against RSV infection, a new experiment was performed at Sigmovir Biosystems, Inc, following the study design presented in FIG. 4. A unique dose of 150 μg was chosen for Viaskin-SVLP-FsII boost immunization since it constitutes a pertinent compromise between 100 and 200 μg doses used in the first study. Moreover, it permits a more rigorous comparison with subcutaneous immunization that was performed at the same dose. As a control for immunopathology, mice were immunized with formalin-inactivated RSV (group 5) that corresponds to the formulation used for the very first clinical trial in the 60^{ies}. As a positive control for protection, mice were infected at day 0 with RSV (group 6). All mice were challenged intranasally with RSV A2 at day 42.

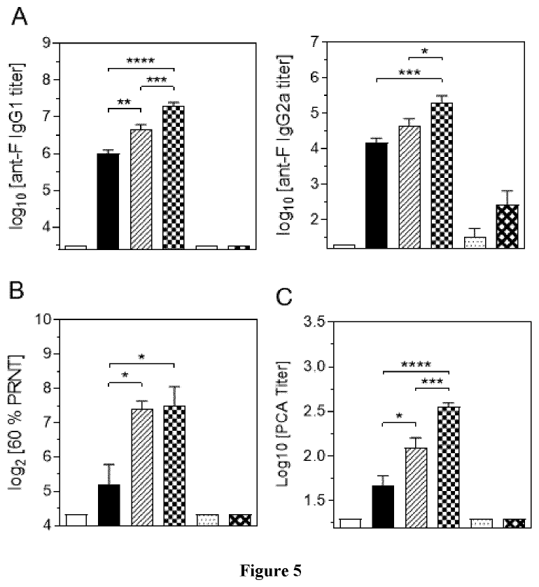

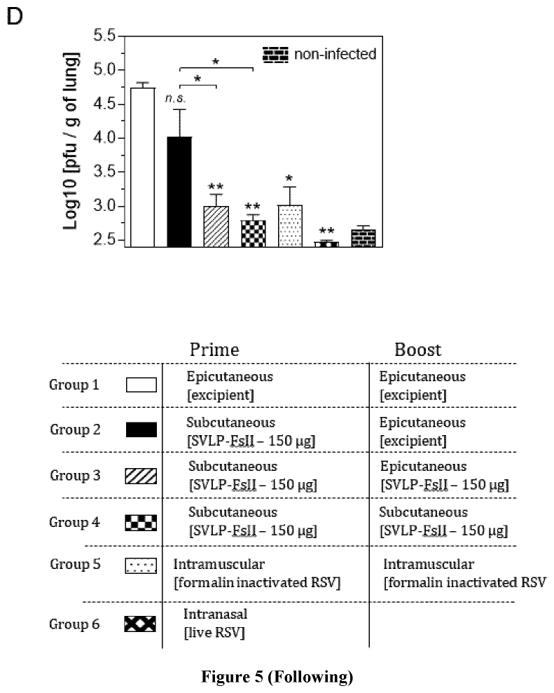

A blood sample was collected at day 42, before RSV challenge, and F-specific IgG1 and IgG2a antibody titers were measured by ELISA (FIG. 5A). A significant increase of F-specific IgG1 was measured for mice boosted epicutaneously with Viaskin-SVLP-FsII compared to mice boosted with Viaskin-excipient patch (mean IgG1 titer of 6.7±0.3 log 10 for Viaskin-SVLP-FsII versus 6.0±0.2 log 10 for Viaskin-excipient, p<0.01; mean IgG2a titer of 4.7±0.5 log 10 for Viaskin-SVLP-FsII versus 4.2±0.3 log 10 for Viaskin-excipient, non-significant). Of note, IgG1 and IgG2a titers obtained for mice boosted epicutaneously with Viaskin-SVLP-FsII were significantly lower than those obtained for mice boosted subcutaneously with SVLP-FsII. However, IgG1/IgG2a ratio was identical between the two groups (mean ratio of 1.4±0.1 for both group) suggesting that the orientation of the immune response was not affected by the route of immunization. As expected, formalin-inactivated vaccine and RSV infection induced poor anti-F antibody titers.

To validate the functionality and the quality of these antibodies, a neutralization assay and a PCA were performed from sera collected at day 42 (FIGS. 5B and 5C). In line with our previous set of data, a significant increase of RSV-neutralizing and PCA titers was measured from mice boosted epicutaneously with Viaskin-SVLP-FsII compared to mice boosted with Viaskin-excipient (mean neutralization titer of 7.4±0.6 log 10 for Viaskin-SVLP-FsII versus 5.2±1.5 log 10 for Viaskin-excipient, p<0.05; mean PCA titer of 2.1±0.3 log 10 for Viaskin-SVLP-FsII versus 1.7±0.3 log 10 for Viaskin-excipient, p<0.05). Of note, PCA titers obtained from mice boosted epicutaneously with Viaskin-SVLP-FsII were significantly lower than those obtained from mice boosted subcutaneously with SVLP-FsII. However, RSV neutralizing titres were found similar. As expected, and in agreement with the low F-specific antibody titers measured by ELISA, formalin-inactivated vaccine and RSV infection did not induce any RSV-neutralizing and PCA antibodies.

In order to evaluate the capacity of epicutaneous boost immunization with Viaskin-SVLP-FsII to give an advantage for protection against RSV infection, mice were challenged 3 weeks after the boost immunization. Five days later, mice were sacrificed, and lungs were collected. A part of each lung was homogenized, and RSV viral load was measured by plate titration (FIG. 5D). A significant decrease of viral load was observed from mice boosted epicutaneously with Viaskin-SVLP-FsII compared to mice boosted with Viaskin-excipient or non-vaccinated mice (mean pfu per gram of lung of 3.0±0.4 log 10 for Viaskin-SVLP-FsII versus 4.0±1.0 log 10 for Viaskin-excipient, p<0.05, versus 4.7±0.1 log 10 for non-vaccinated mice). This decrease was similar to that observed from mice boosted subcutaneously with SVLP-FsII (mean pfu per gram of lung of 2.8±0.2 log 10). As expected, a strong protection was observed from mice vaccinated with formalin-inactivated virus or infected at day 0, probably through the induction of cellular response.

To conclude, these data demonstrate that epicutaneous boost immunization with Viaskin-SVLP-FsII is able to induce neutralizing antibodies, leading to efficient protection against RSV replication in mouse lungs.

4. Epicutaneous Boost Immunization with Viaskin-SVLP-FsII is Safe and does not Exacerbate Lung Inflammation Following RSV Challenge

The main issue related to the first RSV vaccine tested in human (formalin-inactivated virus) was the exacerbation of lung inflammation following RSV infection. This aberrant reaction was due to the poor quality of the immunity induced by the vaccine that was mainly of Th2 orientation.

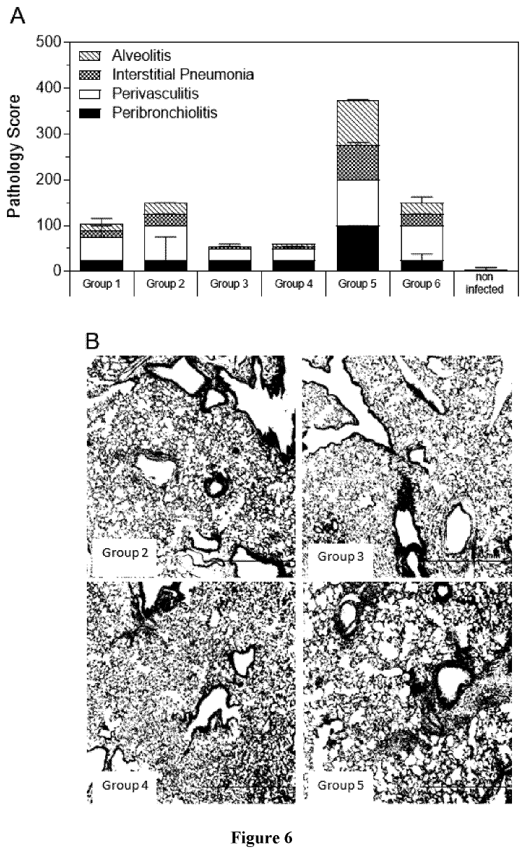

In order to validate the absence of immunopathology in mice boosted epicutaneously with Viaskin-SVLP-FsII, histological sections were performed from lungs collected at day 5 post-infection (day 42). Then, histological slices were coloured by Haematoxylin-Eosin-Safran staining and analysed (FIGS. 6A and 6B). A significant reduction of lung pathology was observed from mice boosted epicutaneously with Viaskin-SVLP-FsII or subcutaneously with SVLP-FsII compared to mice that received formalin-inactivated RSV (p<0.0001 for both criteria) or that were boosted with Viaskin-excipient patch (p<0.01 for perivasculitis). Moreover, lung pathology was not significantly increased or even lower in mice boosted epicutaneously with Viaskin-SVLP-FsII or subcutaneously with SVLP-FsII compared to non-infected mice.

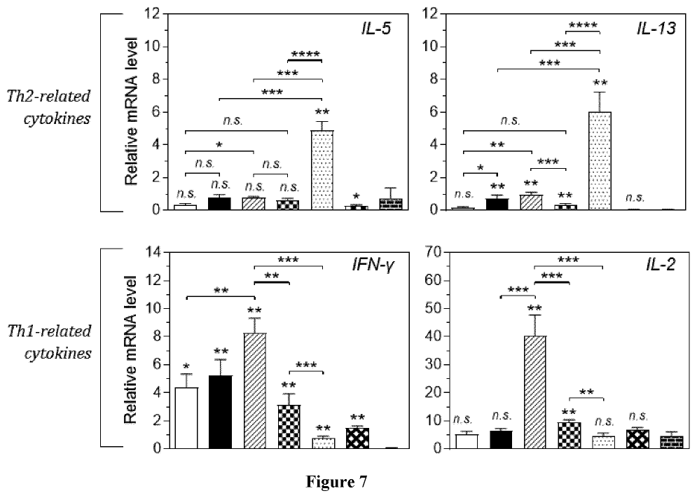

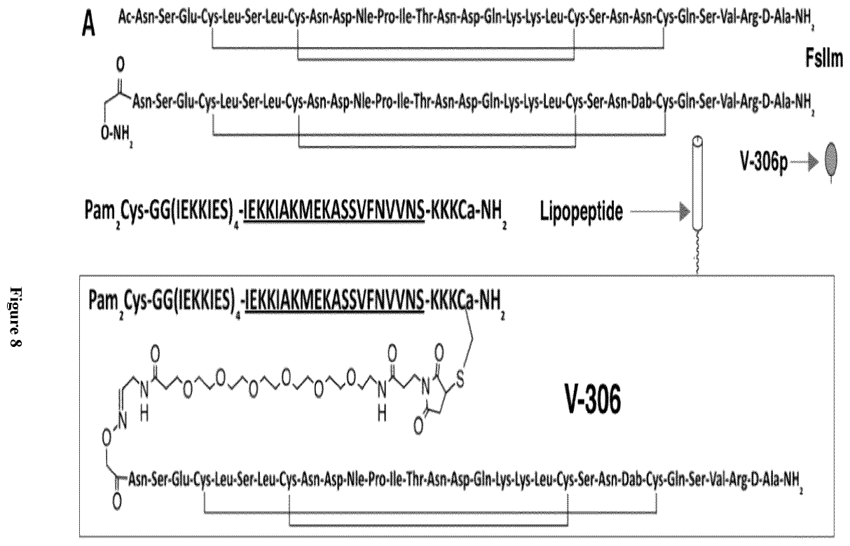

Then, in order to evaluate the orientation of the immune response recalled by RSV infection, mRNA was extracted from lung and analyzed by qPCR to measure the expression of Th1- (IFN-γ; IL-2) or Th2- (IL-5; IL-13) related cytokines (FIG. 7). A strong reduction of the expression of Th2-related cytokines was observed for mice that have been boosted epicutaneously with Viaskin-SVLP-FsII or subcutaneously with SVLP-FsII compared to mice that have been vaccinated with formalin-inactivated RSV (IL-5 and IL-13: p<0.001 and p<0.0001 for epicutaneous and subcutaneous boosts, respectively). Conversely, a strong increase of the expression of Th1-related cytokines was observed for mice that have been boosted epicutaneously with Viaskin-SVLP-FsII or subcutaneously with SVLP-FsII compared to mice that have been vaccinated with formalin-inactivated RSV (IFN-γ: p<0.001 for epicutaneous and subcutaneous boosts; IL-2: p<0.001 and p<0.01 for epicutaneous and subcutaneous boosts, respectively). Of note, this increase was more pronounced in mice that have been boosted epicutaneously with Viaskin-SVLP-FsII, compared to mice that have been boosted subcutaneously with SVLP-FsII, especially for IL-2 (IFN-γ: p<0.01: IL-2: p<0.001). This suggest that epicutaneous route is more efficient than subcutaneous route to promote Th1 local effectors that can be restimulated following infection.

To conclude, these data demonstrate that epicutaneous boost with Viaskin-SVLP-FsII gives protection against RSV replication in lung without inducing inflammation, by promoting the induction of Th1 local effectors.

### CONCLUSIONS

Overall, these results indicate that Viaskin-SVLP-FsII is efficient as an epicutaneous boost vaccination strategy against RSV.

Indeed, the inventors have shown that Viaskin-SVLP-FsII V-306 patch was able to significantly boost FsII antibody titers in mice that have been previously primed subcutaneously with SVLP-FsII V-306. Importantly, these antibodies could efficiently neutralize RSV infectivity and compete with Palivizumab binding in vitro. Of note, this boost effect was almost as efficient or even more efficient as that observed after boosting with a subcutaneous injection of SVLP-FsII V-306.

Even more importantly, epicutaneous boost with Viaskin-SVLP-FsII gave a significant benefit for protection against RSV replication in lung without exacerbating local inflammation. Moreover, epicutaneous boost with Viaskin-SVLP-FsII was able to promote Th1 effectors in lung that were recalled following RSV infection. This Th1 orientation was assessed by the local increase of IFN-γ and IL-2 expression. Of note, IFN-γ and IL-2 expressions were higher in mice boosted by epicutaneous route than in mice boosted by subcutaneous route, suggesting that upper skin is a preferable route to enhance Th1 immunity.

The non-invasive epicutaneous patch would advantageously reduce the number of injections required, especially if repeated boosters are required over the years to maintain a stable level of protective immunity. One possible approach would be to propose a subcutaneous priming dose of V-306 to all women of childbearing age followed by repeated epicutaneous boosters of the same antigen, before and during pregnancy. This may lead to a pronounced increase in the level of RSV-neutralizing antibodies that likely would be transferred to the fetus through the placenta. As assessed by PCA, these antibodies would be analogous to palivizumab, for which the best correlation with protection has been established to date, and for which a low proportion of adverse events have been reported in the prior art.

Epicutaneous patches can also be used for boosting RSV-specific immunity acquired through natural infection. Whilst most adults have experienced several RSV infections during their life, specific immunity is short lived, leading to a high heterogenicity between individuals in terms of protective immunity. In this regard, a non-invasive epicutaneous booster vaccine would be a way to enhance specific humoral immunity by recalling memory B-cells naturally induced by RSV.

### Example 2: An Epitope-Specific Chemically Defined Nanoparticle Vaccine for Respiratory Syncytial Virus

The inventors developed the RSV vaccine V-306 which relates to an SVLP comprising a bundle of conjugates of formula (38). V-306 elicits strong long-lasting RSV-neutralizing antibody responses in mice and rabbits that protect mice from RSV infection and disease enhancement in a validated preclinical RSV challenge model.

Results

1. Design of Epitope Mimetic

Design of a conformationally constrained peptide mimicking the epitope recognized by Motavizumab, led to the FsII site mimetics of SEQ ID NO: 47 and SEQ ID NO: 129 (FsIIm), with stabilizing sequence modifications and cysteines for cyclization via disulfide bridges at specific antigenically non-critical positions, shown in FIG. 8A. The solution structure of this peptide was determined by homonuclear 1H NMR spectroscopy. SEQ ID NO: 47 and SEQ ID NO: 129 (FsIIm) adopt a stable helix-rich folded conformation in water (FIG. 8B). The solution structure superimposed very closely on that of the Motavizumab-bound peptide (PDB 3IXT), showing that it is an excellent structural mimetic of the epitope. Further optimization of the physical and immunological properties led to the mimetic of SEQ ID NO: 45 and V-306p (FIG. 8A, SEQ ID NOs: 85). The latter has been conjugated to a lipopeptide building block resulting in a conjugate of formula (38). SVLPs comprising bundles of three conjugates of formula (38) were assembled (FIG. 8C) and used as RSV vaccine candidates.

2. Construction and Structural Characterization of V-306

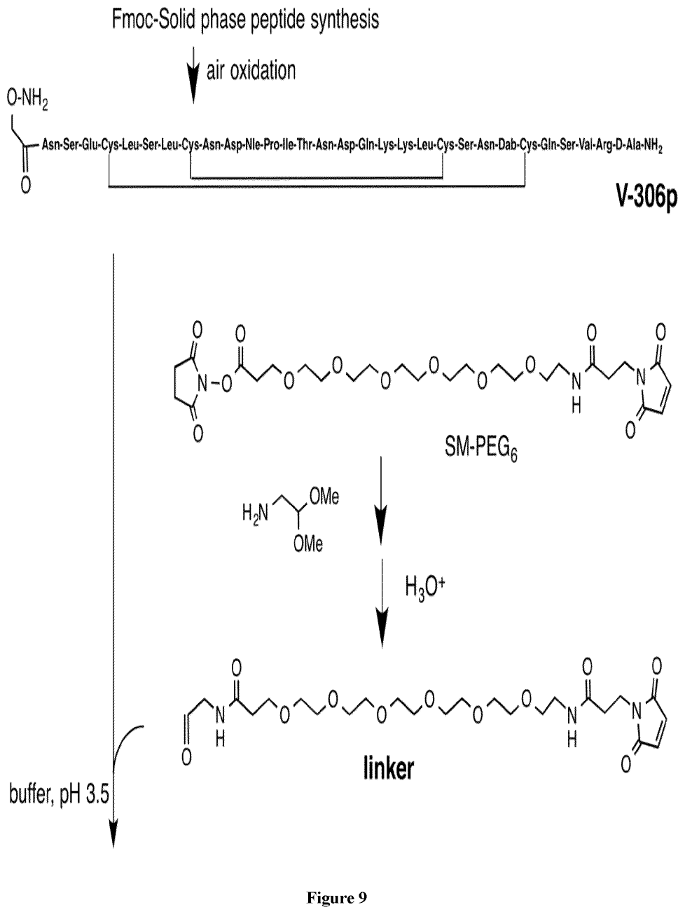

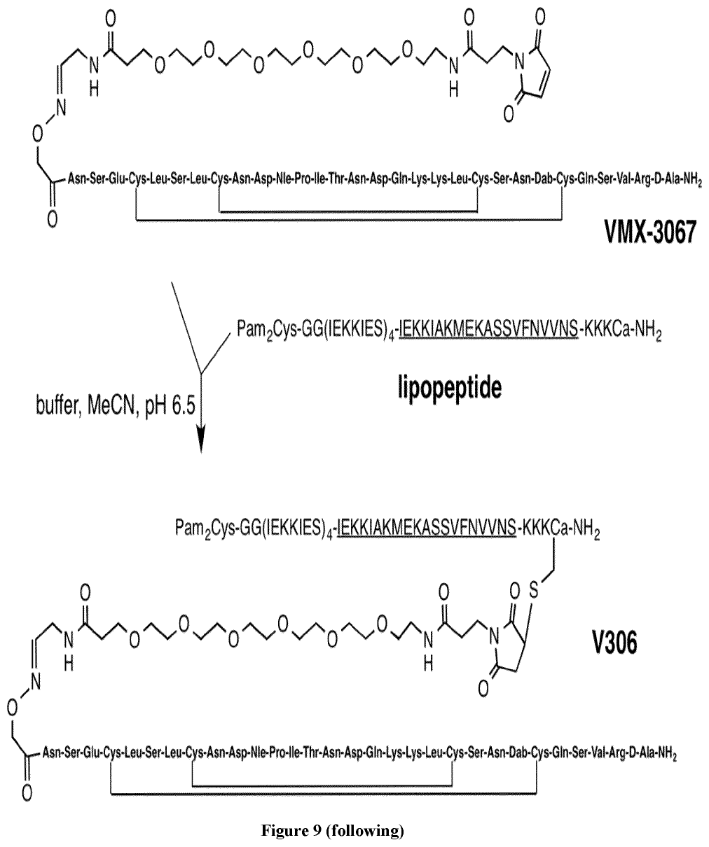

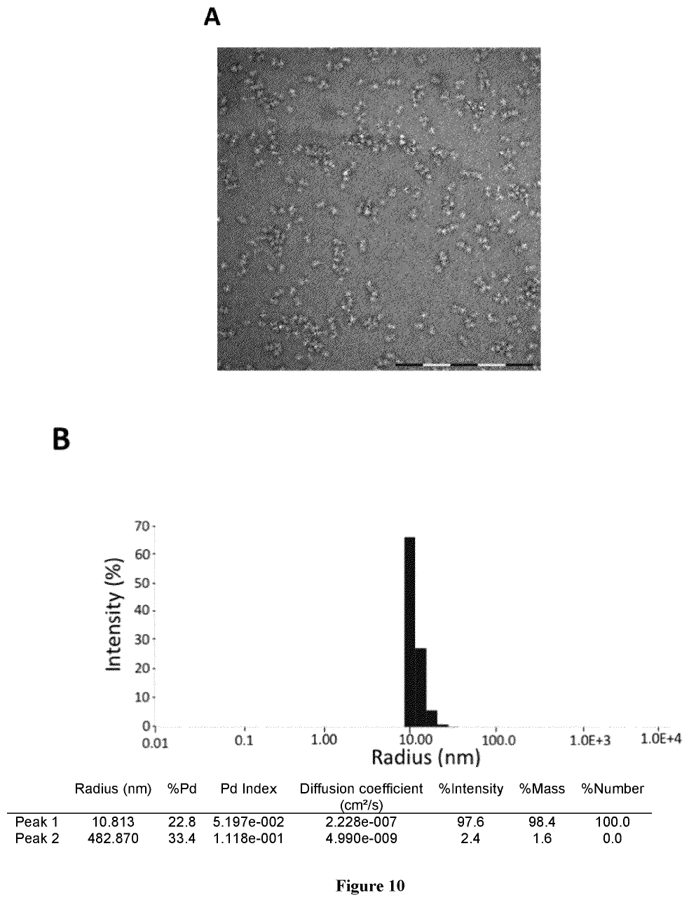

The mimetic V-306p contains an N-terminal aminooxyacetyl group for conjugation to an engineered synthetic lipopeptide (FIG. 8A and FIG. 9) that contains a promiscuous CD4+T helper epitope, a coiled-coil motif (heptad repeat IEKKIES) that forms a very stable helical trimer, and at the N-terminus the TLR-2/6 ligand Pam2Cys. V-306p was linked to this lipopeptide via a maleimide-PEG-aldehyde linker to give the vaccine construct V-306 (FIGS. 8A and 9). The conjugate V-306 in phosphate buffered saline (PBS) was analyzed by Dynamic Light Scattering (DLS) and transmission electron microscopy (FIG. 10). DLS revealed nanoparticle formation in phosphate buffered saline (PBS) with a mean hydrodynamic radius (Rh) of ca. 13 nm and a polydispersity index of 0.05, consistent with formation of highly monodisperse nanoparticles of about 26 nm diameter. Based on computer modelling, about 60-90 copies of each V-306 lipopeptide chain should comprise each nanoparticle, with the lipid chains buried in the core of the micelle-like particle and the epitope mimetic exposed in its surface (depicted in FIG. 8C). Transmission electron microscopy also revealed the formation of nanoparticles in a similar size range 25-30 nm (see FIG. 10) which bound to Palivizumab in ELISA, indicating that the conformational epitope remained intact on the nanoparticle surface.

The V-306p mimetic was then conjugated to a synthetic lipopeptide that contains a coiled-coil domain and a universal T-helper epitope. The resulting conjugate V-306 spontaneously self-assembles into chemically defined micelle-like nanoparticles in PBS with the epitope mimetic displayed in a multivalent format over the surface of the nanoparticle.

Methods

Synthesis of V-306p, FsIIm and further peptides disclosed herein: Peptides were synthesized by solid-phase peptide synthesis using Fmoc-chemistry and Rink amide resin, using procedures known in the prior art. For the synthesis of FsIIm, the completed peptide chain was acetylated at the N-terminus prior to cleavage from the resin and removal of side chain protecting groups, by treatment with trifluoroacetic acid (TFA), thioanisole, H₂O, ethanedithiol (87.5:5:5:2,5) for 2.5 h. The peptide was precipitated and washed with iPr₂O. For oxidation, the reduced peptide was dissolved in 0.33 M ammonium bicarbonate buffer, pH 7.8 and stirred in air overnight. The peptide was purified by reverse phase (RP)-HPLC on a preparative C₁₈ column and lyophilized to afford a white powder. Analytical RP-HPLC (Vydac 218TP54, 5 μm, 4.6 mm×250 mm column, 0-60% MeCN in H₂O (+0.1% TFA) over 40 min): Purity: 90.4%; t_{R}=25.07 min. ESI-MS: Mass calculated for C₁₃₅H₂₂₇N₄₃O₄₉S₄: 3349.52; m/z [M+3H]³⁺ 1117.51.

For the synthesis of V-306 (FIG. 9), Bis-Boc-aminooxyacetic acid N-hydroxysuccinimide ester (Boc₂-Aoa-OSu) was coupled to the N-terminus of the peptide chain. Removal from the resin, deprotection and oxidation to give V-306p were as described above. The disulfide cross-linked peptide was then purified by reverse phase (RP)-HPLC on a preparative C18 column and lyophilized to afford a white powder. Analytical RP-HPLC (Waters BEH C₈, 1.7 μm, 2.1×150 mm column, 10-50% MeCN in H₂O (+0.05% TFA) over 45 min, 0.2 mL/min, 30° C.): Purity: 92.8%; t_{R}=29.95 min. ESI-MS: Mass calculated for C₁₃₅H₂₃₀N₄₄O₄₉S₄: 3379.57 Da; m/z [M+H]⁺: 3380.60 Da (±0.3%).

Linker was prepared by first reacting N-hydroxysuccinimidyl-([N-maleimidopropionamido]-hexa-ethyleneglycol ester (SM-PEG₆, Thermo Fisher Scientific) with aminoacetaldehyde dimethyl acetal (Aldrich) in H₂O. SM-PEG₆ (7.6 mg, 12.6 μmol) was suspended in H₂O (0.3 mL) and a solution of aminoacetaldehyde dimethyl acetal in H₂O (17 μl of a 1:10 (v/v)) was added. The mixture was stirred for 90 min. at room temp. The product was purified by RP-HPLC on a C8 column and lyophilized. Analytical RP-HPLC Waters BEH C₈, 150×2.1 mm, 1.7 μm, 0 to 20% MeCN in H₂O (+0.05% formic acid) over 20 min, 0.4 mL/min, 40° C.: Purity 94.6%, t_{R}=14.29 min. ESI-MS: monoisotopic mass C₂₆H₄₅N₃O₁₂: 591.30 Da; [M+H]⁺ found: 591.62 Da (±0.1%). Just before conjugation, hydrolysis of the dimethyl acetal was performed with 95% TFA, 5% H₂O for 5 min. The TFA was removed in vacuo to give the linker. ESI-MS C₂₄H₃₉N₃O₁₁: 545.26 Da; [M+H]⁺ found: 545.28 Da (±0.05%).

The lipopeptide was synthesized and purified by RP-HPLC as described elsewhere (Boato, F. et al. Angew. Chem. Int. Ed. 46, 9015-9018 (2007), Ghasparian, A. et al. Chembiochem 12, 100-109 (2011), Perriman, A. W. et al. Small 6, 1191-1196 (2010), Sharma, R. et al. J. Immunol. 199, 734-749 (2017)). Analytical RP-HPLC Waters BEH C₈, 150×2.1 mm, 1.7 μm, 64 to 91% MeOH in H₂O (+0.05% TFA) over 37.5 min, 0.4 mL/min, 70° C.: Purity 97.0%, t_{R}=21.80 min. ESI-MS: monoisotopic mass C₃₁₂H₅₅₂N₇₄O₈₉S₃: 6856.0 Da; m/z [M+H]⁺ found 6860.0 Da (±0.05%).

To prepare V-306, a solution of peptide V-306p (12 mg, 3.6 μmol) in 0.25 ml 0.1 M sodium acetate buffer, pH 3.5 was added to linker (3.8 mg, 7.2 μmol) in 0.25 ml 0.1 M sodium acetate buffer, pH 3.5. The mixture was stirred for 2.5 h and the product oxime (called VMX-3067) was purified by RP-HPLC on a preparative C₈ column. Analytical UPLC (Waters BEH C8, 1.7 μm, 2.1×150 mm, 10 to 40% MeCN in H₂O (+0.05% formic acid) over 37.5 min, 0.4 mL/min, 26° C.: Purity 95%, t_{R}=21.5 min. ESI-MS: mass calculated for C158H263N47O59S4: 3893.32 Da; m/z [M+H]⁺ found 3893.48 (0.3%). The oxime (4.0 mg, 1.0 μmol) was dissolved in 0.5 ml H₂O and added to a solution of lipopeptide (6.2 mg, 0.9 μmol) in 2 ml 50% MeCN. The pH was adjusted to pH=6.5 with 0.1 N NaOH/0.1 N HCl and the mixture was stirred at r.t. for 2.5 h. The conjugate V-306 was purified by RP-HPLC on a C8 column. The TFA salt was converted first to an acetate salt and then to a hydrochloride salt using AG-X2 anion exchange resin. The conjugate was analyzed by analytical UPLC and MS (FIG. 9). UPLC (Waters BEH C8, 1.7 μm, 2.1×150 mm, 20 to 80% MeCN in H₂O (+0.03% TFA) over 60 min, 26° C.: Purity 90%, t_{R}=51.5 min. ESI-MS: Monoisotopic mass calc. for C470H815N121O148S7: 10746.9 Da; m/z [M+13H]13+ found 827.6875 Da (±0.1%). Conjugate V-306 was suspended in PBS, equilibrated for 30 minutes, diluted to 1.0 mg/ml and analyzed by Dynamic Light Scattering (DLS) on a DynaPro Nanostar instrument (Wyatt Technology) at 25° C. The size distribution by regularization analysis was monomodal. The mean hydrodynamic radius (Rh) was ca. 13 nm, and the polydispersity (Pd) index was 0.038.

### Example 3: Preparation of Epicutaneous Patches

V-306p-conjugated lipopeptide lyophilizate was dissolved to a concentration of 2 mg/mL in sterile PBS 1× for reconstitution and incubated 30 min at room temperature (RT). During this time, solution was gently mixed by vortex for 1 min every 10 min to ensure the formation of SVLPs (V-306). Then, 50, 75 or 100 μL (100, 150 or 200 μg, respectively) of V-306 solution was deposited on Viaskin® patches (DBV Technologies). Patches were dried in a ventilated oven. One day before patch application, mice were anaesthetized with ketamine and xylazine (50 and 10 mg/kg, respectively) and hair was removed from the back using electric clippers and depilatory cream. Patches were applied on the depilated back (intact skin) and secured using a bandage for 48 h.

### Example 4: V-306 SVLP Stability on Epicutaneous Patch

To initially validate the compatibility of V-306 with the epicutaneous patch, and to evaluate the stability of the combined product, 100 or 200 μg of V-306 SVLP were loaded on patches and further stored for 1 week at 4° C., 1 month at 4° C. and 1 month at RT. Then, V-306 SVLP was recovered from the patches using water and analyzed by DLS and UPLC/MS. The totality of V-306 could be recovered from patches stored 1 week at 4° C. Furthermore, this recovery leads to the formation of nanoparticles with 20-25 nm size, identical to reference V-306 SVLP. The recovery rate was slightly lower for patches stored 1 month at 4° C., and even lower for patches stored 1 month at RT, suggesting a partial degradation of V-306 over time (Table 1). However, at least half of the loaded material could be recovered, leading to the formation of well-shaped SVLPs. To investigate the capacity of trans-epidermal water loss to dissolve V-306 from patches in vivo, patches containing 100 μg of V-306 SVLP were applied to mice for 48 h and analyzed as described above by DLS and UPLC/MS (n=2). No remaining material could be retrieved from these patches, suggesting that the whole deposit was dissolved by skin humidity and the totality of antigen reached the upper layer of the skin

Characterization of V-306 nanoparticles following recovery from epicutaneous patches:

| | | | | Hydro- | |
|---|---|---|---|---|---|
| | | | | dynamic | |
| | | Duration | | radius of | |
| | Storage | of | Recovery^{a} | particles | |
| Material | conditions | storage | [%] | [nm] | Identity |
| Patch [100 μg] | 4° C. | 1 Week | 122 | 10.73 | Complies |
| Patch [200 μg] | 4° C. | 1 Week | 101 | 11.37 | Complies |
| Patch [100 μg] | 4° C. | 1 Month | 71 | 10.43 | Complies |
| Patch [200 μg] | 4° C. | 1 Month | 85 | 11.12 | Complies |
| Patch [100 μg] | RT | 1 Month | 44 | 11.13 | Complies |
| Patch [200 μg] | RT | 1 Month | 52 | 11.84 | Complies |
| Reference | NA | NA | NA | 10.05 | NA |
| V-306 | | | | | |

a Recovery was defined as the percentage of protein quantity recovered from patch compared to the actual quantity loaded on patch. b Identity was assessed using UPLC/MS by comparing the molecular weight of the protein recovered from patch to the molecular weight of reference V-306 material.

1. A particle comprising an RSV-F protein, a variant or a fragment thereof for use in a method of prevention of a disease caused by RSV, by epicutaneous vaccination with said particle.

2. A particle comprising an RSV-F protein, a variant or a fragment thereof for use in a method for vaccinating an infant against RSV by maternal epicutaneous vaccination with said particle.

3. The particle for use according to claim 1 or 2, wherein said vaccination leads to the generation of neutralizing antibodies directed against RSV-F protein in a subject treated with said particle.

4. The particle for use according to any one of claim 1 to 3, wherein said fragment of said RSV-F protein is a sequence selected from the group consisting of SEQ ID NO: 2, 3, 4, 5, and wherein said variant of said RSV-F protein is a cyclic peptide comprising an amino acid sequence (I), wherein said amino acid sequence (I) comprises, preferably consists of, the amino acid sequence:
(SEQ ID NO: 44)
X1-X2-X3-C4-X5-X6-X7-C8-X9-X10-X11-P12-I13-T14-
N15-D16-Q17-K18-K19-L20-C21-X22-X23-X24-C25-X26-
X27-X28-X29-X30,
wherein X1, X2, X3, X5, X6, X7, X9, X10, X11, X22, X23, X24, X26, X27, X28 and X29 are independently of each other an amino acid;
C4, C8, C21 and C25 are independently of each other cysteine;
P12 is proline;
113 is isoleucine;
T14 is threonine;
N15 is asparagine;
D16 is aspartic acid;
Q17 is glutamine;
K18 and K19 are independently of each other lysine;
L20 is leucine; and
X30 is an amino acid or a deletion,
wherein said cysteines C4 and C25 form a first disulfide bond and said cysteines C8 and C21 form a second disulfide bond.

5. The particle for use according to claim 4, wherein said amino acid sequence (I) comprises or preferably consists of an amino acid sequence selected from the group consisting of any one of SEQ ID NO: 45-88.

6. The particle for use according to claims 1 to 5, wherein said particle is applied epicutaneously to an infant of less than 6 months.

7. The particle for use according to any one of claims 1 to 6, wherein said particle is applied epicutaneously to a pregnant female during the second and third quarters of the pregnancy, preferably during the second quarter or a mother during lactation.

8. The particle for use according to any one of claims 1 to 7, wherein said particle is a synthetic virus-like-particle (SVLP).

9. The particle for use according to claim 8, wherein said SVLP consists of conjugates, wherein each conjugate comprises, preferably consists of:
a peptide chain comprising a coiled coil-domain and optionally a T-helper epitope,
a lipid moiety comprising two or three, preferably two, hydrocarbyl chains, and
said RSV-F protein, said variant or said fragment thereof,
wherein the peptide chain is linked to said RSV-F protein, said variant or said fragment thereof and to the lipid moiety.

10. The particle for use according to claim 8 or 9, wherein:
said peptide chain comprises a coiled coil peptide chain segment comprising 3 to 8 repeat units, preferably 4 repeat units, wherein said repeat unit consists of the sequence IEKKIE-X0 (SEQ ID NO: 115), wherein X0 represents an amino acid, preferably said repeat unit consists of the sequence selected from IEKKIEG (SEQ ID NO: 116), IEKKIEA (SEQ ID NO: 117) or IEKKIES (SEQ ID NO:118), more preferably said repeat unit consists of the sequence IEKKIES (SEQ ID NO:118);
said lipid moiety comprises the formula LM-II, 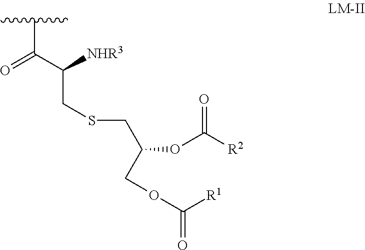
wherein R¹ and R² are independently C₁₁₋₁₅alkyl, preferably R¹ and R² are independently —C₁₁H₂₃, —C₁₃H₂₇ or —C₁₅H₃₁, and further preferably R¹ and R² are —C₁₅H₃₁; R³ is hydrogen or —C(O)C₁₁₋₁₅alkyl, preferably R³ is H or —C(O)C₁₅H₃₁; and wherein the wavy line in formula LM-II indicates the linkage site of said lipid moiety to said peptide chain; and
said RSV-F protein, said variant or said fragment thereof is a sequence selected from group consisting of SEQ ID NO: 2-5 and 45-88, preferably SEQ ID NO: 45-88, more preferably SEQ ID NO: 45 or 85.

11. The particle for use according to claim 10, wherein said conjugate is selected from any one of the formulae (38), (39), (40), (41) or (42), wherein preferably said conjugate is of formula (38)

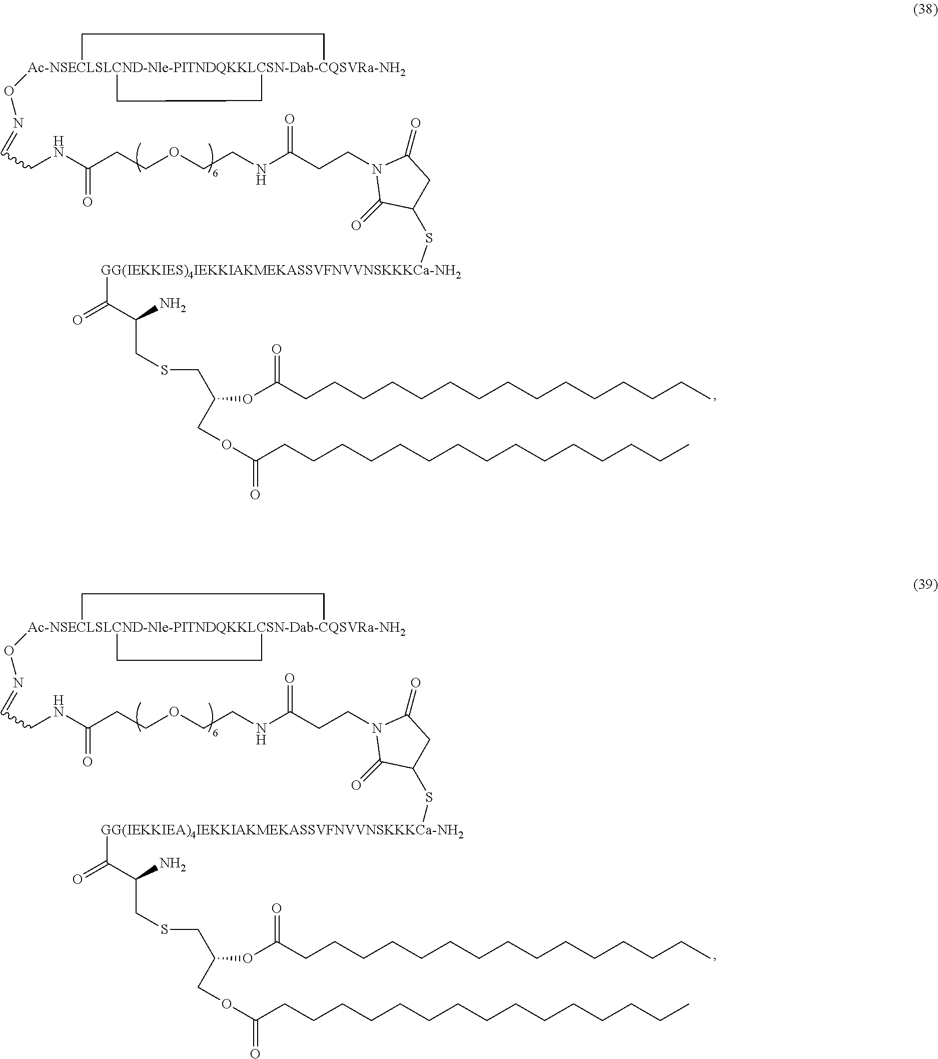

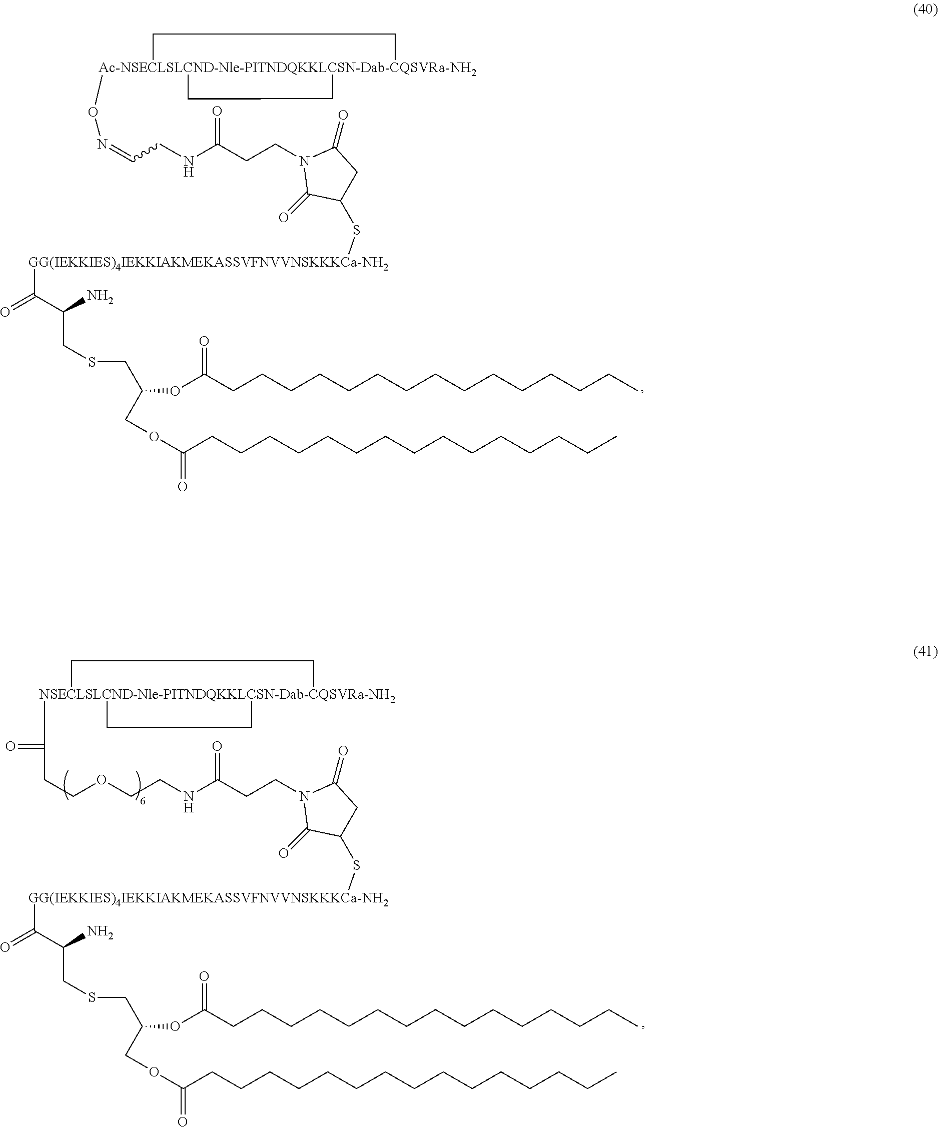

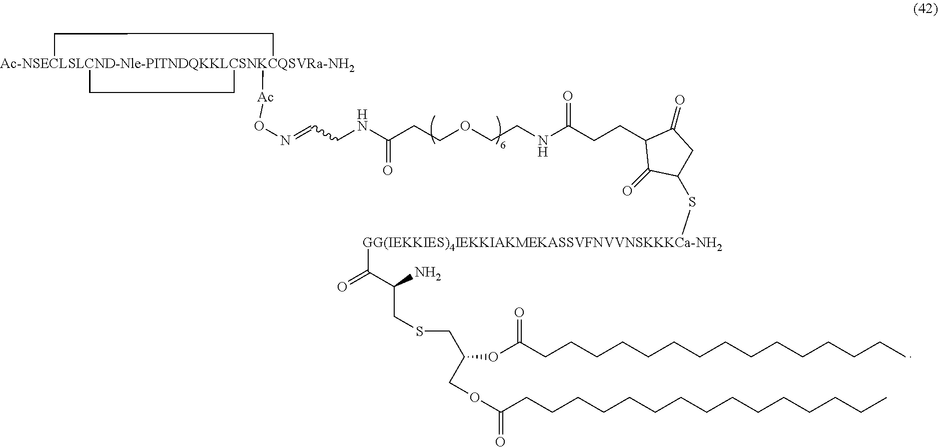

12. The particle for use according to anyone of claims 1 to 11, wherein said particle is applied using a skin patch device, preferably an electrostatic skin patch device.

13. The particle for use according to claim 12, wherein said epicutaneous vaccination is performed by the application of said skin patch device on a pretreated skin.

14. A method for preparing a skin patch device comprising depositing, preferably by electrospraying, at least one particle comprising an RSV F protein, a variant or a fragment thereof on a surface of a skin patch device.

15. A skin patch device comprising an application surface, wherein the application surface contains an SVLP comprising an RSV-F protein, a variant or a fragment thereof.

16. Use of a particle comprising an RSV-F protein, a variant or a fragment thereof in the manufacture of a drug for the prevention of a disease caused by RSV, wherein the drug is delivered by means of a skin patch by epicutaneous route to provide vaccination against RSV.

17. Use of a particle comprising an RSV-F protein, a variant or a fragment thereof in the manufacture of a drug for passively vaccinating an infant against RSV, wherein the drug is administered by means of a skin patch by epicutaneous route to the infant's mother.

18. The use according to claim 16, wherein the drug is epicutaneously administered to an infant of less than 6 months.

19. The use according to claim 17, wherein the drug is epicutaneously administered to the mother during the second or third quarters of the pregnancy, preferably during the second quarter or during breastfeeding period.

20. The use according to any one of claims 16 to 19, wherein the particle is as defined in any one of claims 4, 5 and 8-11.

21. A method for providing vaccination against RSV to a subject which comprises administering the subject with a particle comprising an RSV-F protein, a variant or a fragment thereof by epicutaneous route, preferably by means of a skin patch.

22. A method for providing passive vaccination to an infant against RSV, which comprises administering the infant's mother with a particle comprising an RSV-F protein, a variant or a fragment thereof by epicutaneous route, preferably by means of a skin patch.

23. The method according to claim 21, wherein the drug is epicutaneously administered to an infant of less than 6 months.

24. The method according to claim 22, wherein the drug is epicutaneously administered to the mother during the second and third quarters of the pregnancy, preferably during the second quarter or during breastfeeding period.

25. The method according to any one of claims 21 to 24 wherein the particle is as defined in any one of claims 4, 5 and 8-11.


SEQUENCE LISTING (130 sequences)

SEQ ID NO: 1 - protein, 574 aa, respiratory syncytial virus
MELPILKANAITTILAAVTFCFASSQNITEEFYQSTCSAVSKGYLSALRTGWYTSVITIELSNIKENKCNGTDAKVKLINQELDKYKNAVTELQLLMQSTTAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 2 - protein, 24 aa, Artificial Sequence
  Antigenic Site II - 1
NSELLSLINDMPITNDQKKLMSNN

SEQ ID NO: 3 - protein, 21 aa, Artificial Sequence
  Antigenic Site II - 2
SELLSLINDMPITNDQKKLMS

SEQ ID NO: 4 - protein, 22 aa, Artificial Sequence
  Antigenic Site II - 3
NSELLSLINDMPITNDQKKLMS

SEQ ID NO: 5 - protein, 12 aa, Artificial Sequence
  Antigenic site IV
CTASNKNRGIIK

SEQ ID NO: 6 - protein, 52 aa, influenza virus hemagglutinin
GSTQAAIDQINGKLNRVIEKTNEKFHQIEKEFSEVEGRIQDLEKYVEDTKCG

SEQ ID NO: 7 - protein, 46 aa, human immuno-deficiency virus
SGIVQQQNNLLRAIEAQQHLLQLTVWGIKQLQARILAVERYLGDCG

SEQ ID NO: 8 - protein, 55 aa, bovine immuno-deficiency virus
GGERWQNVSYIAQTQDQFTHLFRNINNRLNVLHHRVSYLEYVEEIRQKQVFFGCG

SEQ ID NO: 9 - protein, 56 aa, feline immuno-deficiency virus
GGATHQETIEKVTEALKINNLRLVTLEHQVLVIGLKVEAMEKFLYTAFAMQELGCG

SEQ ID NO: 10 - protein, 56 aa, equine infectious anemia virus
GGNHTFEVENSTLNGMDLIERQIKILYAMILQTHARVQLLKERQQVEETFNLIGCG

SEQ ID NO: 11 - protein, 57 aa, simian immuno-deficiency virus
GGAQSRTLLAGIVQQQQQLLDWKRQQELLRLTVWGTKNLQTRVTAIEKYLKDQAGCG

SEQ ID NO: 12 - protein, 52 aa, caprine arthritis encephalitis virus
GGSYTKAAVQTLANATAAQQDVLEATYAMVQHVAKGVRILEARVARVEAGCG

SEQ ID NO: 13 - protein, 55 aa, Visna virus
GGSLANATAAQQNVLEATYAMVQHVAKGIRILEARVARVEAIIDRMMVYQELDCG

SEQ ID NO: 14 - protein, 54 aa, Artificial Sequence
  human parainfluenza-3
GGEAKQARSDIEKLKEAIRDTNKAVQSVQSSIGNLIVAIKSVQDYVNKEIVGCG

SEQ ID NO: 15 - protein, 54 aa, human parainfluenza-1
GGEAREARKDIALIKDSIIKTHNSVELIQRGIGEQIIALKTLQDFVNNEIRGCG

SEQ ID NO: 16 - protein, 55 aa, human parainfluenza-2
GGKANANAAAINNLASSIQSTNKAVSDVITASRTIATAVQAIQDHINGAIVNGCG

SEQ ID NO: 17 - protein, 53 aa, human parainfluenza-4a
GGKAQENAKLILTLKKAATETNEAVRDLANSNKIWKMISAIQNQINTIIQGCG

SEQ ID NO: 18 - protein, 54 aa, human parainfluenza-4b
GGKAQENAQLILTLKKAAKETNDAVRDLTKSNKIVARMISAIQNQINTIIQGCG

SEQ ID NO: 19 - protein, 53 aa, Measles virus
GGSMLNSQAIDNLRASLETTNQAIEAIRQSGQEMILAVQGVQDYINNELIGCG

SEQ ID NO: 20 - protein, 59 aa, Mumps virus
GGAQTNARAIAAMKNSIQATNRAVFEVKEGTQQLAIAVQAIQDHINTIMNTQLNNMSCG

SEQ ID NO: 21 - protein, 54 aa, Bovine respiratory syncytial virus
GGAVSKVLHLEGEVNKIKNALLSTNKAWSLSNGVSVLTSKVLDLKNYIDKEGCG

SEQ ID NO: 22 - protein, 48 aa, Ebola virus
GGANETTQALQLFLRATTELRTFSILNRKAIDFLLQRWGGTCHILGCG

SEQ ID NO: 23 - protein, 48 aa, Marburg virus
GGANQTAKSLELLLRVTTEERTFSLINRHAIDFLLTRWGGTCKVLGCG

SEQ ID NO: 24 - protein, 43 aa, Rous sarcoma virus
GGANLTTSLLGDLLDDVTSIRHAVLQNRAAIDFLLLAHGHGCG

SEQ ID NO: 25 - protein, 55 aa, Staphylothermus marinus
GSIINETADDIVYRLTVIIDDRYESLKNLITLRADRLEMIINDNVSTILASIGCG

SEQ ID NO: 26 - protein, 61 aa, SARS coronavirus
GGNVLYENQKQIANQFNKAISQIQESLTTTSTALGKLQDWNQNAQALNTLVKQLSSNFGCG

SEQ ID NO: 27 - protein, 66 aa, Mycoplasma pneumoniae
GGTKTEFKEFQTWMESFAVQNQNIDAQGEQIKELQVEQKAQGKTLQLILEALQGINKRLDNLESCG

SEQ ID NO: 28 - protein, 38 aa, Artificial Sequence
  heptameric coiled coil
GGKVKQLADAVEELASANYHLANAVARLAKAVGERGCG

SEQ ID NO: 29 - protein, 53 aa, Artificial Sequence
  trimeric coiled coil
GGIEKKIEAIEKKIEAIEKKIEAIEKKIEAIEKKIAKMEKASSVFNWNSKKKC

SEQ ID NO: 30 - protein, 45 aa, Artificial Sequence
  tetrameric coiled coil
KLKQIEDKLEEILSKLYHIENELAKIEKKLAKMEKASSVFNWKKC

SEQ ID NO: 31 - protein, 18 aa, Artificial Sequence
  universal T helper cell epitope
  MISC_FEATURE (1)..(1): X is H-Asp-Ile-, H-Ile- or H-,
  MISC_FEATURE (18)..(18): X is -Val-Asn-Ser-OH, -Val-Asn-OH, -Val-OH or -OH
XEKKIAKMEKASSVFNVX

SEQ ID NO: 32 - protein, 18 aa, Artificial Sequence
  T helper cell epitope
DIEKKIAKMEKASSVFNV

SEQ ID NO: 33 - protein, 19 aa, Artificial Sequence
  T helper cell epitope
DIEKKIAKMEKASSVFNVV

SEQ ID NO: 34 - protein, 20 aa, Artificial Sequence
  T helper cell epitope
DIEKKIAKMEKASSVFNVVN

SEQ ID NO: 35 - protein, 17 aa, Artificial Sequence
  T helper cell epitope
IEKKIAKMEKASSVFNV

SEQ ID NO: 36 - protein, 18 aa, Artificial Sequence
  T helper cell epitope
IEKKIAKMEKASSVFNVV

SEQ ID NO: 37 - protein, 19 aa, Artificial Sequence
  T helper cell epitope
IEKKIAKMEKASSVFNVVN

SEQ ID NO: 38 - protein, 20 aa, Artificial Sequence
  T helper cell epitope
IEKKIAKMEKASSVFNVVNS

SEQ ID NO: 39 - protein, 16 aa, Artificial Sequence
  T helper cell epitope
EKKIAKMEKASSVFNV

SEQ ID NO: 40 - protein, 16 aa, Artificial Sequence
  T helper cell epitope
EKKIAKMEKASSVFNV

SEQ ID NO: 41 - protein, 18 aa, Artificial Sequence
  T helper cell epitope
EKKIAKMEKASSVFNVVN

SEQ ID NO: 42 - protein, 19 aa, Artificial Sequence
  T helper cell epitope
EKKIAKMEKASSVFNVVNS

SEQ ID NO: 43 - protein, 21 aa, Artificial Sequence
  T helper cell epitope
DIEKKIAKMEKASSVFNVVNS

SEQ ID NO: 44 - protein, 30 aa, Artificial Sequence
  cyclic protein
  MISC_FEATURE (1)..(3): Amino acid Xaa
  MISC_FEATURE (5)..(7): Amino acid Xaa
  MISC_FEATURE (9)..(11): Amino acid Xaa
  MISC_FEATURE (22)..(24): Amino acid Xaa
  MISC_FEATURE (26)..(30): Amino acid Xaa
XXXCXXXCXXXPITNDQKKLCXXXCXXXXX

SEQ ID NO: 45 - protein, 30 aa, Artificial Sequence
  cyclic protein
  MOD_RES (11)..(11): Nle
  MOD_RES (24)..(24): Dbu
  MISC_FEATURE (30)..(30): D-alanine
NSECLSLCNDXPITNDQKKLCSNXCQSVRX

SEQ ID NO: 46 - protein, 30 aa, Artificial Sequence
  cyclic protein
  MOD_RES (11)..(11): Nle
  MISC_FEATURE (30)..(30): D-alanine
NSECLSLCNDXPITNDQKKLCSNKCQSVRX

SEQ ID NO: 47 - protein, 30 aa, Artificial Sequence
  cyclic protein
  MOD_RES (11)..(11): Nle
  MISC_FEATURE (30)..(30): D-alanine
NSECLSLCNDXPITNDQKKLCSNNCQSVRX

SEQ ID NO: 48 - protein, 30 aa, Artificial Sequence
  cyclic protein
  MOD_RES (11)..(11): Nle
  MISC_FEATURE (30)..(30): D-alanine
NSECLSLCNDXPITNDQKKLCSNDCQSVRX

SEQ ID NO: 49 - protein, 30 aa, Artificial Sequence; identical to SEQ ID NO: 45
  cyclic protein
  MOD_RES (11)..(11): Nle
  MOD_RES (24)..(24): Orn
  MISC_FEATURE (30)..(30): D-alanine

SEQ ID NO: 50 - protein, 30 aa, Artificial Sequence
  cyclic protein
  MOD_RES (12)..(12): Nle
  MISC_FEATURE (30)..(30): D-alanine
RLSECLSLCNDXPITNDQKKLCSNNCLKSX

SEQ ID NO: 51 - protein, 40 aa, Artificial Sequence
  cyclic protein
  MISC_FEATURE (40)..(40): D-alanine
PVSTYMLTNSECLSLCNDMPITNDQKKLCSNNCQIVRQQX

SEQ ID NO: 52 - protein, 30 aa, Artificial Sequence
  cyclic protein
  MOD_RES (12)..(12): Nle
  MISC_FEATURE (30)..(30): D-alanine
RLSECLSLCNDXPITNDQKKLCSNKCLKSX

SEQ ID NO: 53 - protein, 30 aa, Artificial Sequence
  cyclic protein
  MOD_RES (12)..(12): Nle
  MOD_RES (25)..(25)
  MISC_FEATURE (30)..(30): D-alanine
RLSECLSLCNDXPITNDQKKLCSNXCLKSX

SEQ ID NO: 54 - protein, 40 aa, Artificial Sequence
  cyclic protein
  MISC_FEATURE (40)..(40): D-alanine
PVSTYMLTNSECLSLCNDMPITNDQKKLCSNKCQIVRQQX

SEQ ID NO: 55 - protein, 40 aa, Artificial Sequence
  cyclic protein
  MOD_RES (32)..(32): Dbu
  MISC_FEATURE (40)..(40): D-alanine
PVSTYMLTNSECLSLCNDMPITNDQKKLCSNXCQIVRQQX

SEQ ID NO: 56 - protein, 30 aa, Artificial Sequence
  cyclic protein
  MOD_RES (12)..(12): Nle
  MISC_FEATURE (30)..(30): D-alanine
RLSECLSLCNDXPITNDQKKLCSNDCLKSX

SEQ ID NO: 57 - protein, 30 aa, Artificial Sequence; identical to SEQ ID NO: 53
  cyclic protein
  MOD_RES (12)..(12): Nle
  MOD_RES (25)..(25): Orn
  MISC_FEATURE (30)..(30): D-alanine

SEQ ID NO: 58 - protein, 40 aa, Artificial Sequence
  cyclic protein
  MISC_FEATURE (40)..(40): D-alanine
PVSTYMLTNSECLSLCNDMPITNDQKKLCSNDCQIVRQQX

SEQ ID NO: 59 - protein, 40 aa, Artificial Sequence; identical to SEQ ID NO: 55
  cyclic protein
  MOD_RES (32)..(32): Orn
  MISC_FEATURE (40)..(40): D-alanine

SEQ ID NO: 60 - protein, 40 aa, Artificial Sequence
  cyclic protein
  MOD_RES (19)..(19): Nle
  MISC_FEATURE (40)..(40): D-alanine
PVSTYMLTNSECLSLCNDXPITNDQKKLCSNNCQIVRQQX

SEQ ID NO: 61 - protein, 40 aa, Artificial Sequence
  cyclic protein
  MOD_RES (19)..(19): Nle
  MISC_FEATURE (40)..(40): D-alanine
PVSTYMLTNSECLSLCNDXPITNDQKKLCSNKCQIVRQQX

SEQ ID NO: 62 - protein, 40 aa, Artificial Sequence
  cyclic protein
  MOD_RES (19)..(19): Nle
  MOD_RES (32)..(32): Dbu
  MISC_FEATURE (40)..(40): D-alanine
PVSTYMLTNSECLSLCNDXPITNDQKKLCSNXCQIVRQQX

SEQ ID NO: 63 - protein, 40 aa, Artificial Sequence
  cyclic protein
  MOD_RES (19)..(19): Nle
  MISC_FEATURE (40)..(40): D-alanine
PVSTYMLTNSECLSLCNDXPITNDQKKLCSNDCQIVRQQX

SEQ ID NO: 64 - protein, 40 aa, Artificial Sequence; identical to SEQ ID NO: 62
  cyclic protein
  MOD_RES (19)..(19): Nle
  MOD_RES (32)..(32): Orn
  MOD_RES (40)..(40): D-alanine

SEQ ID NO: 65 - protein, 30 aa, Artificial Sequence
  cyclic protein
  MOD_RES (11)..(11): Nle
  MOD_RES (24)..(24): Dbu
  MISC_FEATURE (30)..(30): D-alanine
NSECLSLCNDXPITNDQKKLCSSXCQSVRX

SEQ ID NO: 66 - protein, 30 aa, Artificial Sequence
  cyclic protein
  MOD_RES (11)..(11): Nle
  MISC_FEATURE (30)..(30): D-alanine
NSECLSLCNDXPITNDQKKLCSSNCQSVRX

SEQ ID NO: 67 - protein, 30 aa, Artificial Sequence
  cyclic protein
  MOD_RES (11)..(11): Nle
  MISC_FEATURE (30)..(30): D-alanine
NSECLSLCNDXPITNDQKKLCSSQCQSVRX

SEQ ID NO: 68 - protein, 30 aa, Artificial Sequence
  cyclic protein
  MOD_RES (11)..(11): Nle
  MOD_RES (30)..(30): AMIDATION
  MISC_FEATURE (30)..(30): D-alanine
NSECLSLCNDXPITNDQKKLCSSSCQSVRX

SEQ ID NO: 69 - protein, 30 aa, Artificial Sequence
  cyclic protein
  MOD_RES (11)..(11): Nle
  MOD_RES (24)..(24): Dbu
  MISC_FEATURE (30)..(30): D-alanine
QSECLSLCNDXPITNDQKKLCSNXCQSVRX

SEQ ID NO: 70 - protein, 30 aa, Artificial Sequence
  cyclic protein
  MOD_RES (11)..(11): Nle
  MOD_RES (24)..(24): Dbu
  MISC_FEATURE (30)..(30): D-alanine
QSECLSLCNDXPITNDQKKLCSSXCQSVRX

SEQ ID NO: 71 - protein, 30 aa, Artificial Sequence
  cyclic protein
  MOD_RES (11)..(11): Nle
  MISC_FEATURE (30)..(30): D-alanine
QSECLSLCNDXPITNDQKKLCSSNCQSVRX

SEQ ID NO: 72 - protein, 30 aa, Artificial Sequence
  cyclic protein
  MOD_RES (11)..(11): Nle
  MISC_FEATURE (30)..(30): D-alanine
QSECLSLCNDXPITNDQKKLCSSQCQSVRX

SEQ ID NO: 73 - protein, 30 aa, Artificial Sequence
  cyclic protein
  MOD_RES (11)..(11): Nle
  MISC_FEATURE (30)..(30): D-alanine
QSECLSLCNDXPITNDQKKLCSSSCQSVRX

SEQ ID NO: 74 - protein, 30 aa, Artificial Sequence
  cyclic protein
  MOD_RES (11)..(11): Nle
  MOD_RES (24)..(24): Dbu
  MISC_FEATURE (30)..(30): D-alanine
SSECLSLCNDXPITNDQKKLCSNXCQSVRX

SEQ ID NO: 75 - protein, 30 aa, Artificial Sequence
  cyclic protein
  MOD_RES (11)..(11): Nle
  MOD_RES (24)..(24): Dbu
  MISC_FEATURE (30)..(30): D-alanine
SSECLSLCNDXPITNDQKKLCSSXCQSVRX

SEQ ID NO: 76 - protein, 30 aa, Artificial Sequence
  cyclic protein
  MOD_RES (11)..(11): Nle
  MISC_FEATURE (30)..(30): D-alanine
SSECLSLCNDXPITNDQKKLCSSNCQSVRX

SEQ ID NO: 77 - protein, 30 aa, Artificial Sequence
  cyclic protein
  MOD_RES (11)..(11): Nle
  MISC_FEATURE (30)..(30): D-alanine
SSECLSLCNDXPITNDQKKLCSSQCQSVRX

SEQ ID NO: 78 - protein, 30 aa, Artificial Sequence
  cyclic protein
  MOD_RES (11)..(11): Nle
  MISC_FEATURE (30)..(30): D-alanine
SSECLSLCNDXPITNDQKKLCSSSCQSVRX

SEQ ID NO: 79 - protein, 30 aa, Artificial Sequence
  cyclic protein
  MOD_RES (11)..(11): Nle
  MOD_RES (24)..(24): Dbu
  MISC_FEATURE (30)..(30): D-alanine
GSECLSLCNDXPITNDQKKLCSNXCQSVRX

SEQ ID NO: 80 - protein, 30 aa, Artificial Sequence
  cyclic protein
  MOD_RES (11)..(11): Nle
  MOD_RES (24)..(24): Dbu
  MISC_FEATURE (30)..(30): D-alanine
GSECLSLCNDXPITNDQKKLCSSXCQSVRX

SEQ ID NO: 81 - protein, 30 aa, Artificial Sequence
  cyclic protein
  MOD_RES (11)..(11): Nle
  MISC_FEATURE (30)..(30): D-alanine
GSECLSLCNDXPITNDQKKLCSSNCQSVRX

SEQ ID NO: 82 - protein, 30 aa, Artificial Sequence
  cyclic protein
  MOD_RES (11)..(11): Nle
  MISC_FEATURE (30)..(30): D-alanine
GSECLSLCNDXPITNDQKKLCSSQCQSVRX

SEQ ID NO: 83 - protein, 30 aa, Artificial Sequence
  cyclic protein
  MOD_RES (11)..(11): Nle
  MISC_FEATURE (30)..(30): D-alanine
GSECLSLCNDXPITNDQKKLCSSSCQSVRX

SEQ ID NO: 84 - protein, 30 aa, Artificial Sequence; identical to SEQ ID NO: 45
  cyclic protein
  BINDING (1)..(1): AoAc moiety incl. 1-amino-3,6,9,12,15, 18-hexaoxaheneicosan-21-oic amide linker
  MOD_RES (11)..(11): Nle
  MOD_RES (24)..(24): Dbu
  MOD_RES (30)..(30): Amidation
  MISC_FEATURE (30)..(30): D-alanine

SEQ ID NO: 85 - protein, 30 aa, Artificial Sequence; identical to SEQ ID NO: 45
  cyclic protein
  BINDING (1)..(1): AoAc moiety
  MOD_RES (11)..(11): Nle
  MOD_RES (24)..(24): Dbu
  MISC_FEATURE (30)..(30): D-alanine
  MOD_RES (30)..(30)
  MOD_RES (30)..(30): Amidation

SEQ ID NO: 86 - protein, 30 aa, Artificial Sequence; identical to SEQ ID NO: 46
  cyclic protein
  BINDING (1)..(1): Ac moiety
  MOD_RES (11)..(11): Nle
  BINDING (24)..(24): AOAc moiety
  MOD_RES (30)..(30): Amidation
  MISC_FEATURE (30)..(30): D-alanine

SEQ ID NO: 87 - protein, 30 aa, Artificial Sequence; identical to SEQ ID NO: 50
  cyclic protein
  BINDING (1)..(1): AOAc
  MOD_RES (12)..(12): Nle
  MOD_RES (30)..(30): Amidation
  MISC_FEATURE (30)..(30): D-alanine

SEQ ID NO: 88 - protein, 40 aa, Artificial Sequence; identical to SEQ ID NO: 51
  cyclic protein
  BINDING (1)..(1): AOAc
  MISC_FEATURE (40)..(40): D-alanine
  MOD_RES (40)..(40): Amidation

SEQ ID NO: 89 - protein, 20 aa, Artificial Sequence
  CS.T3
IEKKIAKMEKASSVFNVVNS

SEQ ID NO: 90 - protein, 14 aa, Artificial Sequence
  TT830-843
QYIKANSKFIGITE

SEQ ID NO: 91 - protein, 16 aa, Artificial Sequence
  TT1064-1079
IREDNNITLKLDRCNN

SEQ ID NO: 92 - protein, 15 aa, Artificial Sequence
  TT1084-1099
VSIDKFRIFCKANPK

SEQ ID NO: 93 - protein, 22 aa, Artificial Sequence
  TT947-968
FNNFTVSFWLRVPKVSASHLET

SEQ ID NO: 94 - protein, 16 aa, Artificial Sequence
  TT1174-1189
LKFIIKRYTPNNEIDS

SEQ ID NO: 95 - protein, 20 aa, Artificial Sequence
  DTD271-290
PVFAGANYAAWAVNVAQVID

SEQ ID NO: 96 - protein, 20 aa, Artificial Sequence
  DTD321-340
VHHNTEEIVAQSIALSSLMV

SEQ ID NO: 97 - protein, 20 aa, Artificial Sequence
  DTD331-350
QSIALSSLMVAQAIPLVGEL

SEQ ID NO: 98 - protein, 20 aa, Artificial Sequence
  DTD351-370
VDIGFAAYNFVESIINLFQV

SEQ ID NO: 99 - protein, 20 aa, Artificial Sequence
  DTD411-430
QGESGHDIKITAENTPLPIA

SEQ ID NO: 100 - protein, 20 aa, Artificial Sequence
  DTD431-450
GVLLPTIPGKLDVNKSKTHI

SEQ ID NO: 101 - protein, 20 aa, Artificial Sequence
  TT632-651
TIDKISDVSTIVPYIGPALN

SEQ ID NO: 102 - protein, 25 aa, Artificial Sequence
  CTMOMP36-60
ALNIWDRFDVFCTLGATTGYLKGNS

SEQ ID NO: 103 - protein, 14 aa, Artificial Sequence
  TraT1
GLQGKIADAVKAKG

SEQ ID NO: 104 - protein, 19 aa, Artificial Sequence
  TraT2
GLAAGLVGMAADAMVEDVN

SEQ ID NO: 105 - protein, 20 aa, Artificial Sequence
  TraT3
STETGNQHHYQTRVVSNANK

SEQ ID NO: 106 - protein, 20 aa, Artificial Sequence
  HbcAg50-69
PHHTALRQAILCWGELMTLA

SEQ ID NO: 107 - protein, 15 aa, Artificial Sequence
  HbSAg19-33
FFLLTRILTIPQSLD

SEQ ID NO: 108 - protein, 13 aa, Artificial Sequence
  HA307-319
PKYVKQNTLKLAT

SEQ ID NO: 109 - protein, 16 aa, Artificial Sequence
  MA17-31
YSGPLKAEIAQRLEDV

SEQ ID NO: 110 - protein, 20 aa, Artificial Sequence
  MVF258-277
GILESRGIKARITHVDTESY

SEQ ID NO: 111 - protein, 15 aa, Artificial Sequence
  MVF288-302
LSEIKGVIVHRLEGV

SEQ ID NO: 112 - protein, 17 aa, Artificial Sequence
  SM Th
KWFKTNAPNGVDEKIRI

SEQ ID NO: 113 - protein, 13 aa, Artificial Sequence
  PADRE1
  misc_feature (1)..(1): D-alanine
  misc_feature (13)..(13): D-alanine
AKFVAAWTLKAAA

SEQ ID NO: 114 - protein, 13 aa, Artificial Sequence
  PADRE2
  MISC_FEATURE (1)..(1): D-alanine
  MOD_RES (3)..(3): cyclohexylalanine
  MISC_FEATURE (13)..(13): D-alanine
AKXVAAWTLKAAA

SEQ ID NO: 115 - protein, 7 aa, Artificial Sequence
  repeat unit
  MISC_FEATURE (7)..(7): any amino acid
IEKKIEX

SEQ ID NO: 116 - protein, 7 aa, Artificial Sequence
  repeat unit
IEKKIEG

SEQ ID NO: 117 - protein, 7 aa, Artificial Sequence
  repeat unit
IEKKIEA

SEQ ID NO: 118 - protein, 7 aa, Artificial Sequence
  repeat unit
IEKKIES

SEQ ID NO: 119 - protein, 28 aa, Artificial Sequence
  coiled coil
IEKKIEGIEKKIEGIEKKIEGIEKKIEG

SEQ ID NO: 120 - protein, 28 aa, Artificial Sequence
  coiled coil
IEKKIEAIEKKIEAIEKKIEAIEKKIEA

SEQ ID NO: 121 - protein, 28 aa, Artificial Sequence
  coiled coil
IEKKIESIEKKIESIEKKIESIEKKIES

SEQ ID NO: 122 - protein, 10 aa, Artificial Sequence
  FSL-1
  BINDING (1)..(1): Pam-2
CGDPKHPKSF

SEQ ID NO: 123 - protein, 10 aa, Artificial Sequence
  FSL-2
  Binding (1)..(1): Pam-2
CGDPKHPKSR

SEQ ID NO: 124 - protein, 10 aa, Artificial Sequence
  FSL-3
  binding (1)..(1): S-(2,3-bisstearyloxypropyl)-
CGDPKHPKSF

SEQ ID NO: 125 - protein, 14 aa, Artificial Sequence
  MALP-2
  Binding (1)..(1): PAM2-
CGNNDESNISFKEK

SEQ ID NO: 126 - protein, 5 aa, Artificial Sequence
  KKKCa
  MISC_FEATURE (5)..(5): D-alanine
KKKCA

SEQ ID NO: 127 - protein, 54 aa, Artificial Sequence
  peptide moiety
GGIEKKIESIEKKIESIEKKIESIEKKIESIEKKIAKMEKASSVFNVVNSKKKC

SEQ ID NO: 128 - protein, 54 aa, Artificial Sequence
  peptide moiety
GGIEKKIEAIEKKIEAIEKKIEAIEKKIEAIEKKIAKMEKASSVFNVVNSKKKC

SEQ ID NO: 129 - protein, 56 aa, Artificial Sequence
  peptide moiety
  misc_feature (56)..(56): D-alanine
CGGIEKKIESIEKKIESIEKKIESIEKKIESIEKKIAKMEKASSVFNVVNSKKKCA

SEQ ID NO: 130 - protein, 30 aa, Artificial Sequence; identical to SEQ ID NO: 47
  cyclic protein
  MOD_RES (1)..(1): ACETYLATION
  MOD_RES (11)..(11): Nle
  MOD_RES (30)..(30): Amidation
  MISC_FEATURE (30)..(30): D-alanine